(19) United States
(12) Patent Application Publication
(10) Pub. No.: US 2011/0230478 A1
(43) Pub. Date: Sep. 22, 2011

(54) 4-Alkyl-substituted diaminopyrimidines

(75) Inventors: Jörg Nico Greul, Leichlingen (DE); Oliver Gaertzen, Koin (DE); Hendrik Helmke, Liederbach (DE); Stefan Hillebrand, Neuss (DE); Amos Mattes, langenfeld (DE); Pierre Wasnaire, Dusseldorf (DE); Carl Friedrich Nising, Langenfeld (DE); Urike Wachendorff-Neumann, Neuwied (DE); Peter Dahmen, Neuss (DE); Arnd Voerste, Koin (DE); Ruth Meissner, Leverkusen (DE); Christoph Andreas Braun, Dusseldorf (DE); Martin Kaubmann, Koin (DE); Hiroyuki Hadano, Tochigi (JP); Marcel Calleja, Lyon (FR)

(73) Assignee: Bayer ScienceCrop AG, Monheim (DE)

(21) Appl. No.: 13/061,984

(22) PCT Filed: Aug. 26, 2009
(86) PCT No.: PCT/EP2009/006159

§ 371 Date: May 16, 2011

(30) Foreign Application Priority Data
Sep. 3, 2008 (EP) 08163610.2

(51) Int. Cl. A01N 43/84; A01N 43/54; C07D 239/48; A01N 43/60; C07D 403/12; C07D 413/12; A01P 3/00

(52) U.S. Cl. 514/230.8; 514/275; 544/323; 514/252.14; 544/295; 544/122

(57) ABSTRACT

Use of 4-alkyl-substituted diaminopyrimidines of the formula (I) as fungicide

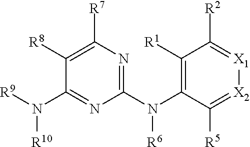

in which R¹ to R¹³ and X¹ and X² have the meanings given in the description, and agrochemically active salts thereof, and also methods and compositions for controlling phytopathogenic harmful fungi in and/or on plants or in and/or on seed of plants, processes for preparing such compositions and treated seed and also their use for controlling phytopathogenic harmful fungi in agriculture, horticulture and forestry, in the protection of materials and in the domestic and hygiene field. The present invention furthermore relates to a process for preparing diaminopyrimidines of the formulae (Ia), (Ib) and (Ic).

The invention relates to 4-alkyl-substituted diaminopyrimidines and their agrochemically active salts, to their use and to methods and compositions for controlling phytopathogenic harmful fungi in and/or on plants or in and/or on seed of plants, to processes for preparing such compositions and treated seeds and also to their use for controlling phytopathogenic harmful fungi in agriculture, horticulture and forestry, in the protection of materials and in the domestic and hygiene field. The present invention furthermore relates to a process for preparing 4-alkyl-substituted diaminopyrimidines.

It is already known that certain alkynyl-substituted diaminopyrimidines can be used as fungicidal crop protection agents (see DE 4029650 A1). However, in particular at low application rates, the fungicidal activity of these compounds is not always sufficient.

Since the ecological and economic demands made on modern fungicides are increasing constantly, for example with respect to activity spectrum, toxicity, selectivity, application rate, formation of residues and favourable manufacture, and there can furthermore be problems, for example, with resistance, there is a constant need to develop novel crop protection agents, in particular fungicides which, at least in some areas, have advantages over the known crop protection agents.

Surprisingly, it has now been found that the present 4-alkyl-substituted diaminopyrimidines solve at least some aspects of the objects mentioned and are suitable for use as crop protection agents, in particular as fungicides.

Some of these 4-alkyl-substituted diaminopyrimidines are already known as pharmaceutically active compounds (see, for example, WO 07/003,596, WO 06/087230, WO 05/111022, WO 05/111023, WO 05/037800, WO 04/065378, WO 04/056786, WO 04/056807, WO 04/048343, WO 03/032997, WO 03/030909, WO 02/096888), but not their surprising fungicidal activity.

The invention provides the use of compounds of the formula (I) as fungicide

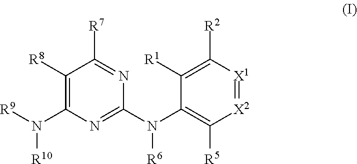

in which the symbols are as defined below:
X¹ represents nitrogen or CR³,
X² represents nitrogen or CR⁴,
where X¹ and X² do not both represent nitrogen,
R¹ and R⁵ independently of one another represent hydrogen, C₁-C₄-alkyl, C₁-C₄-alkoxy or halogen,
R² represents hydrogen, halogen, nitro, cyano, C₁-C₄-alkyl, C₁-C₂-haloalkyl, hydroxyl, OR¹², OCON(R¹²)₂, COR¹², CR¹²═NOR¹², SF₅, SR¹², SOR¹², SO₂R¹², CO₂R¹², N(R¹²)₂, NR¹²COR¹² or NR¹²CO₂R¹³,
R³ and R⁴ independently of one another represent hydrogen, halogen, cyano, nitro, a 3- to 8-membered, unsubstituted or substituted, saturated or unsaturated monocycle which contains no or up to four heteroatoms selected from the group consisting of N, O and S, where two oxygen atoms are not adjacent, OR¹², OCON(R¹²)₂, OCOR¹³, SF₅, SR¹², SOR¹², SO₂R¹², SO₂N(R¹³)₂, C═OR¹², CO(CH₂)ₘCN, CR¹²═NOR¹², CON(R¹²)₂, COOR¹², NR¹²COR¹², NR¹²CSR¹³, NR¹²COOR¹³, NR¹²(C═S)OR¹³, N(R¹²)₂, NR¹²SO₂R¹³, [C(R¹²)₂]ₘCN, (CH₂)ₘSON(R¹²)₂, (CH₂)ₘSO₂N(R¹²)₂, (CR¹²)ₘSO₂N(R¹²)₂, (CH₂)ₘCOOR¹², (CH₂)ₘCOR¹², [C(R¹²)²]ₘCOR¹², (CH₂)ₘCON(R¹²)₂, [C(R¹²)₂]ₘCON(R¹²)₂, (CH₂)ₘNR¹²COR¹², (CH₂)ₘNR(¹²)₂, (CH₂)ₘNR¹²COOR¹³, [C(R¹²)₂]ₘNR¹²COR¹², [C(R¹²)₂]ₘNR¹²COOR¹³, unsubstituted or substituted C₁-C₈-alkyl, C₂-C₆-alkenyl, C₁-C₈-haloalkyl; where m=1-4,
   where additionally R³ and R⁴, if appropriate via R¹² and R¹³, together may form a 5- to 7-membered, unsubstituted or substituted, saturated or unsaturated monocycle which may contain no or up to four heteroatoms selected from the group consisting of N, O and S, where two oxygen atoms are not adjacent,
   where the substituents independently of one another are selected from the group consisting of hydrogen, fluorine, chlorine and bromine, C₁-C₄-alkyl, C₁-C₄-alkoxy, hydroxyl, oxo, C₁-C₄-haloalkyl, (C₁-C₄-alkyl)carbonyl or cyano,
R⁶ represents hydrogen, C₁-C₃-alkyl, C₁-C₄-alkoxy-(C₁-C₄)alkyl, formyl, (C₁-C₄-alkyl)carbonyl, (C₁-C₄-alkoxy-C₁-C₄-alkyl)carbonyl, (C₃-C₆-alkenyloxy)carbonyl, (C₃-C₆-cycloalkyl)carbonyl, (halo-C₁-C₄-alkoxy-C₁-C₄-alkyl)carbonyl, (C₁-C₄-haloalkyl)carbonyl, (C₁-C₄-alkoxy)carbonyl, (C₁-C₄-haloalkoxy)carbonyl, benzyloxycarbonyl, unsubstituted or substituted benzyl, unsubstituted or substituted benzoyl, unsubstituted or substituted C₂-C₆-alkenyl, unsubstituted or substituted C₂-C₆-alkynyl, C₁-C₂-alkylsulphinyl or C₁-C₂-alkylsulphonyl,
   where the substituents independently of one another are selected from the group consisting of hydrogen, fluorine, chlorine and bromine, C₁-C₄-alkyl, C₁-C₄-alkoxy, hydroxyl, C₁-C₄-haloalkyl and cyano,
R⁷ represents hydrogen, C₁-C₃-alkyl, cyano or C₁-C₃-haloalkyl,
R⁸ represents chlorine, bromine, iodine, cyano, methyl, SMe, SOMe, SO₂Me, CF₃, CCl₃, CFH₂ or CF₂H,
R⁹ represents hydrogen, C₁-C₃-alkyl, C₁-C₄-alkoxy-(C₁-C₄)alkyl, (C₁-C₄-alkyl)carbonyl, (C₁-C₄-haloalkyl)carbonyl, (C₁-C₄-alkoxy)carbonyl or optionally fluorine-, chlorine-, bromine-, cyano- or CF₃-substituted C₂-C₄-alkenyl,
R¹⁰ represents a group E1 of the formula

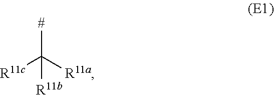

in which the symbols are as defined below and # represents the point of attachment to the nitrogen atom:
R^{11a} represents hydrogen or methyl,
R^{11b} represents hydrogen or C₁-C₃-alkyl,
R^{11c} represents hydrogen, C₁-C₄-alkyl, CN, CH₂CN, CH(Me)CN, C(Me)₂CN, CH₂OH, CH₂O—(C₁-C₃-alkyl), CH₂O—(C₁-C₃-haloalkyl), C(Me)HOH, C(Me)₂OH, C(Me)HO—(C₁-C₃-alkyl), C(Me)₂O—(C₁-C₃-alkyl), C(Me)HO—(C₁-C₃-haloalkyl), C(Me)₂O—(C₁-C₃-haloalkyl), CH₂S—(C₁-C₃-alkyl), C(Me)HS—(C₁-C₃-alkyl), C(Me)₂S—(C₁-C₃-alkyl), Si(C₁-C₂-alkyl)₃ or C₂-C₄-alkenyl which is optionally substituted by halogen or CF₃;
   or
R¹⁰ represents a cyclopentyl or a cyclohexyl ring which may optionally be mono- or polysubstituted by CN, halogen, C₁-C₃-alkyl, C₁-C₃-alkoxy, C₁-C₃-haloalkyl, C₁-C₃-haloalkoxy or C₁-C₃-alkoxy-C₁-C₃-alkyl,
R¹² are identical or different and are hydrogen, C₁-C₆-alkyl, C₁-C₆-haloalkyl, unsubstituted or substituted C₃-C₆-cycloalkyl, C₁-C₄-trialkylsilyl, unsubstituted or substituted C₂-C₄-alkenyl, unsubstituted or substituted C₂-C₄-alkynyl, unsubstituted or substituted phenyl, C₁-C₄-alkoxy(C₁-C₄)alkyl, unsubstituted or substituted benzyl or a 3- to 7-membered unsubstituted or substituted, saturated or unsaturated cycle which may contain no or up to four heteroatoms selected from the group consisting of N, O and S, where two oxygen atoms are not adjacent,
   or
   if two radicals R¹² are attached to a nitrogen atom, two radicals R¹² may form a 3- to 7-membered unsubstituted or substituted saturated or unsaturated cycle which may contain up to four further heteroatoms selected from the group consisting of N, O and S, where two oxygen atoms are not adjacent,
   or
   if two radicals R¹² are adjacent in the grouping NR¹²COR¹², NR¹²SOR¹², NR¹²SO₂R¹², NR¹², SONR¹², NR¹²SO₂NR¹², two radicals R¹² may form a 3- to 7-membered unsubstituted or substituted saturated or unsaturated cycle which may contain up to four further heteroatoms selected from the group consisting of N, O and S, where two oxygen atoms are not adjacent,
R¹³ are identical or different and are C₁-C₈-alkyl, C₁-C₈-haloalkyl, C₁-C₄-trialkylsilyl, unsubstituted or substituted C₂-C₆-alkenyl, unsubstituted or substituted C₂-C₆-alkynyl, unsubstituted or substituted C₃-C₆-cycloalkyl, unsubstituted or substituted aryl, C₁-C₄-alkoxy-(C₁-C₄)alkyl, unsubstituted or substituted benzyl or a 3- to 7-membered unsubstituted or substituted saturated or unsaturated cycle which may contain no or up to four heteroatoms selected from the group consisting of N, O and S, where two oxygen atoms are not adjacent,
   where two R¹³ may form a 3- to 7-membered unsubstituted or substituted saturated or unsaturated cycle which may contain up to four further heteroatoms selected from the group consisting of N, O and S, where two oxygen atoms are not adjacent,
   and where possible substituents are selected from the list below:
   fluorine, chlorine, bromine, iodine, cyano, nitro, CF₃, CFH₂, CF₂H, C₂F₅, CCl₃, hydroxyl, OMe, OEt, OPr, OisoPr, OBu, OsecBu, OisoBu, OtertBu, O(CH₂)₂OCH₃, O(CH₂)₃OCH₃, O-cyclopentyl, O-phenyl, OCF₃, OCF₂H, OCF₂CF₃, OCF₂CF₂H, SH, SMe, SEt, SCF₃, SCF₂H, SPh, SCF₅, SO₂Me, SO₂CF₃, SOMe, SOEt, CO₂H, CO₂CH₃, CO₂Et, CO₂Pr, CO₂isoPr, CO₂tertBu, COMe, COCF₃, NH₂, NHMe, NMe₂, NHEt, NEt₂, NHPr, NHisoPr, NHnBu, NHtertBu, NHisoBu, NHsecBu, cyclopropylamino, formyl, CH₂CN, CHMeCN, CH₂COCH₃, CH₂OMe, (CH₂)₂OMe, (CH₂)₃OMe, CH₂OH, CH₂SMe, (CH₂)₂SMe, methyl, ethyl, propyl, 1-methylethyl, butyl, 1-methylpropyl, 2-methylpropyl, 1,1-dimethylethyl, cyclopropyl, 1-methoxycyclopropyl, 1-chlorocyclopropyl, cyclobutyl, 3-dimethylbutyl, cyclopentyl, cyclohexyl, cyclohexylmethyl, neopentyl, prop-2-en-1-yl, 1-methylprop-2-en-1-yl, but-3-en-1-yl, (trimethylsilyl)methyl, phenyl, benzyl, —CH₂CH═CH₂, —CH(CH₃)CH═CH₂, —CH₂C≡CH,
   and agrochemically active salts thereof.

The diaminopyridines of the formula (I) according to the invention and their agrochemically active salts are highly suitable for use as pesticides, and in particular for controlling phytopathogenic harmful fungi. The compounds according to the invention mentioned above have in particular strong fungicidal activity and can be used both in crop protection, in the domestic and hygiene field and in the protection of materials.

The compounds of the formula (I) can be present both in pure form and as mixtures of various possible isomeric forms, in particular of stereoisomers, such as E and Z, threo- and erythro-, and also optical isomers, such as R and S isomers or atropisomers, and, if appropriate, also of tautomers. What is claimed are both the E and the Z isomers, and the threo- and erythro-, and also the optical isomers, any mixtures of these isomers, and also the possible tautomeric forms.

Preference is given to using, as fungicides, compounds of the formula (I) in which one or more of the symbols are as defined below:
X¹ represents nitrogen or CR³,
X² represents nitrogen or CR⁴,
   where X¹ and X² do not both represent nitrogen,
R¹ and R⁵ independently of one another represent hydrogen, C₁-C₂-alkyl, C₁-C₂-alkoxy, F, Cl or Br,
R² represents hydrogen, halogen, nitro, cyano, C₁-C₄-alkyl, C₁-C₂-haloalkyl, hydroxyl, C₁-C₄-alkoxy, O—(C₂-C₄-alkenyl), O—(C₂-C₄-alkynyl), OCONH—(C₁-C₄-alkyl), OCON—(C₁-C₄-alkyl)₂, C₁-C₃-haloalkoxy, COH, CO—(C₁-C₄-alkyl), C(—NOH)Me, C(═NO)—(C₁-C₄-alkyl)Me, CH(═NO)—(C₁-C₄-alkyl), SF₅, S—(C₁-C₃)-alkyl, S—(C₁-C₂)-haloalkyl, SO—(C₁-C₄-alkyl), SO₂—(C₁-C₄-alkyl), CO₂—(C₁-C₃-alkyl), NH₂, NH—(C₁-C₄-alkyl), N—(C₁-C₄-alkyl)₂, NHCO—(C₁-C₄-alkyl), NHCOH, NMeCO—(C₁-C₄-alkyl) or NHCO₂—(C₁-C₄-alkyl),
R³ and R⁴ independently of one another represent hydrogen, halogen, cyano, nitro, hydroxyl, O-(C₁-C₄)-alkyl, O—(C₃-C₆-cycloalkyl), O—(C₁-C₄-haloalkyl), OCONH—(C₁-C₄-alkyl), OCON—(C₁-C₄-alkyl)₂, OCO—(C₁-C₄-alkyl), SH, SF₅, S—(C₁-C₃-alkyl), S—(C₁-C₃-haloalkyl), SPh, SO—(C₁-C₄-alkyl), SO₂—(C₁-C₄-alkyl), SO₂—(C₂-C₄-alkenyl), SO₂—(C₂-C₄-alkynyl), SO₂—(C₁-C₂-haloalkyl), SO₂N—(C₁-C₄-alkyl)₂, formyl, CO—(C₁-C₄-alkyl), (C₁-C₃-haloalkyl)carbonyl, COCH₂CN, CONH—(C₁-C₄-alkyl), CON—(C₁-C₄-alkyl)₂, CONH—(C₁-C₃-haloalkyl), CONH—(C₂-C₄-alkenyl), CONH—(C₂-C₄-alkynyl), CONH—(C₃-C₆-cycloalkyl), CONHCH(CH₃)CH₂OCH₃, CONH(CH₂)ₘO—(C₁-C₄-alkyl), CONHPh, piperidin-1-ylcarbonyl, morpholin-4-ylcarbonyl, (4-methylpiperazin-1-yl)carbonyl, pyrrolidin-1-ylcarbonyl, azetidin-1-ylcarbonyl, aziridin-1-ylcarbonyl, hexamethylenimin-1-ylcarbonyl, morpholin-1-ylcarbonyl, thiomorpholin-1-ylcarbonyl, COOH, (C₁-C₄-alkoxy)carbonyl, CO₂(CH₂)ₘO—(C₁-C₄-alkyl), NHCO—(C₁-C₄-alkyl), NHCO—(C₁-C₄-haloalkyl), N—(C₁-C₃-alkyl)-CO—(C₁-C₄-alkyl), N—(C₁-C₃-alkyl)-CS—(C₁-C₄-alkyl), NHCO—(C₂-C₄-alkenyl), NH(C—S)O—(C₁-C₄-alkyl), NHCOPh, NHCO(CH₂)ₘO—(C₁-C₄-alkyl), NHCHO, N—(C₁-C₄-alkyl)CHO, NHCO₂—(C₁-C₄-alkyl), NHCO₂Ph, NHCO₂—(C₁-C₂-haloalkyl), N—(C₁-C₄-alkyl)-CO₂—(C₁-C₄-alkyl), NH₂, NH—(C₁-C₄-alkyl), N—(C₁-C₂-alkyl)₂, cyclopropylamino, NHCH(CH₃)CH₂OCH₃, acetyl(cyclopropyl)amino, [(1-methylcyclopropyl)carbonyl]amino, morpholin-1-yl, morpholin-4-ylmethyl, NHSO₂—(C₁-C₄-alkyl), CH₂CN, CHMeCN, CH₂SO₂NH—(C₁-C₄-alkyl), CH₂SO₂N—(C₁-C₄-alkyl)₂, C(Me)HSO₂N—(C₁-C₄-alkyl)₂, CH₂CO—(C₁-C₄-alkyl), CH(CH₃)COCH₃, CH₂CO—(C₃-C₆-cycloalkyl), CH₂CONH—(C₁-C₄-alkyl), CH₂CO₂—(C₁-C₄-alkyl), CH₂NHCOO—(C₁-C₄-alkyl), CH—NO—(C₁-C₄-alkyl), C(CH₃)—NO—(C₁-C₄-alkyl), CH₂NHCO—(C₁-C₄-alkyl), CH₂NHCO—(C₁-C₄-haloalkyl), C₁-C₆-alkyl, C₃-C₆-cycloalkyl, 1-methoxycyclopropyl, 1-chlorocyclopropyl, C₂-C₆-alkenyl, C₁-C₃-haloalkyl, morpholin-4-ylsulphonyl, (3-methyl-2,5-dioxoimidazolidin-1-yl), 3-methyl-2-oxoimidazolidin-1-yl, (3-methyl-2-oxoimidazolidin-1-yl)methyl, piperidin-1-ylsulphonyl, 1,3-thiazol-2-yl, 1,3-thiazol-4-yl, 3,5-dimethylpiperidin-1-yl, 4-(tert-butoxycarbonyl)piperazin-1-yl, 5-ethoxy-3,4-dimethyl-1H-pyrazol-1-yl, acetyl(cyclo-hexyl)amino, 2-furoylamino, 5-ethoxy-3-(trifluoromethyl)-1H-pyrazol-1-yl, 3-(2-chloroethyl)-2-oxoimidazolidin-1-yl, 1,1-dioxidoisothiazolidin-2-yl, 1,1-dioxidotetrahydro-thiophen-2-yl, 4-methyl-5-oxo-4,5-dihydro-1H-1,2,4-triazol-1-yl, 3-methyl-5-oxo-2,5-dihydro-1H-pyrazol-1-yl, 2-(methoxymethyl)pyrrolidin-1-yl, 2-oxocyclopentyl, 2-oxotetrahydrofuran-3-yl, 1-methyl-3-oxo-2,3-dihydro-1H-pyrazol-4-yl, 1-methyl-3-oxopyrazolidin-4-yl, tetrahydrofuran-2-yl, furan-2-yl, 5-methyl-1,1-dioxido-1,2,5-thiadiazolidin-2-yl, (benzylsulphamoyl)methyl, (furan-2-ylcarbonyl)amino, [(1-phenylethyl)sulphamoyl]methyl, 5-oxo-3-(propan-2-yl)-4,5-dihydro-1H-pyrazol-1-yl, 5-oxo-3-(trifluoromethyl)-4,5-dihydro-1H-pyrazol-1-yl, 1H-tetrazol-5-yl, (cyclopropyl-carbonyl)amino, 4,4-dimethyl-2,5-dioxoimidazolidin-1-yl, 2,3-dimethyl-5-oxo-2,5-dihydro-1H-pyrazol-1-yl, 5-thioxo-4,5-dihydro-1H-tetrazol-1-yl, 3-(1-methylethyl)-2-oxoimidazolidin-1-yl, 3-(2-methylpropyl)-2-oxoimidazolidin-1-yl, 2-oxo-3-prop-2-en-1-ylimidazolidin-1-yl, 3-tert-butyl-2-oxoimidazolidin-1-yl, pyrrolidin-1-ylsulphonyl, 2,5-dioxoimidazolidin-4-yl, (morpholin-4-ylsulphonyl)methyl, (piperidin-1-ylsulphonyl)-methyl, (pyrrolidin-1-ylsulphonyl)methyl, 2-oxoimidazolidin-1-yl, 3-methyl-5-oxo-4,5-dihydro-1H-pyrazol-1-yl, 3,4-dimethyl-5-oxo-4,5-dihydro-1H-pyrazol-1-yl, 1-methyl-cyclopentyl, pyrrolidin-1-yl, piperidin-1-yl, 2-oxo-2,5-dihydro-1H-pyrrol-1-yl, 3,3-dimethyl-2-oxocyclopentyl, 3-oxo-4,5-dimethyl-2,4-dihydro-pyrazol-2-yl, 3-oxo-4-ethyl-5-methyl-2,4-dihydropyrazol-2-yl, 3-oxo-5-trifluoromethyl-2,4-dihydropyrazol-2-yl, 3-oxo-5-isopropyl-2,4-dihydropyrazol-2-yl, 3,5-dioxo-4,4-dimethylpyrazolidin-1-yl, 3,5-dioxo-4-ethylpyrazolidin-1-yl, 3-oxo-4,4-dimethylpyrazolidin-1-yl, 2,5-dioxo-2,5-dihydro-1H-pyrrol-1-yl, 3-oxobutyl, 4-methoxy-2-oxo-2,5-dihydro-1H-pyrrol-1-yl, 2-oxo-2,5-dihydro-1H-pyrrol-1-yl, 5-oxo-4,5-dihydro-1H-imidazol-1-yl, 1,1-dioxido-1,2-thiazinan-2-yl, 6-methyl-1,1-dioxido-1,2,6-thiadiazinan-2-yl, cyclopropyl(trifluoroacetyl)amino, where m=1-3,
   and, if R³ and R⁴, if appropriate via R¹² or R¹³, form a cycle, the following subunit from the general formula (I):

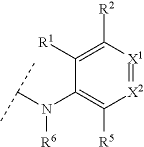

may be (2-oxo-2,3-dihydro-1H-indol-5-yl)amino, 1H-indol-6-ylamino, 1H-indol-5-ylamino, 2-[(trifluoromethyl)-1H-benzimidazol-6-yl]amino, (3-methyl-1,1-dioxido-2H-1,2,4-benzothiadiazin-7-yl)amino, (1,1-dioxido-2H-1,2,4-benzothiadiazin-6-yl)amino, (4-methyl-3-oxo-3,4-dihydro-2H-1,4-benzoxazin-6-yl)amino, (4-methyl-3-oxo-3,4-dihydro-2H-1,4-benzoxazin-7-yl)amino, (1-acetyl-2,3-dihydro-1H-indol-6-yl)amino, (4H-1,3-benzodioxin-7-yl)amino, (2-oxo-2,3,4,5-tetrahydro-1H-1-benzazepin-8-yl)amino, (2,2-dioxido-1,3-dihydro-2-benzothien-5-yl)amino, (1-oxo-2,3-dihydro-1H-inden-5-yl)amino, [2-(ethylsulphonyl)-2,3-dihydro-1,3-benzothiazol-6-yl]amino, (2,2,3,3-tetrafluoro-2,3-dihydro-1,4-benzodioxin-6-yl)amino, 1,3-benzodioxol-5-ylamino, (1,3-dioxo-2,3-dihydro-1H-isoindol-5-yl)amino, (2-methyl-1,3-benzothiazol-6-yl)amino, (2-oxo-2,3-dihydro-1H-benzimidazol-5-yl)amino, (2-oxo-1,3-benzoxathiol-5-yl)amino, (2-oxo-2,3-dihydro-1,3-benzoxazol-5-yl)amino, (2-ethyl-1,3-benzoxazol-5-yl)amino, (2-oxo-1,2,3,4-tetrahydroquinolin-6-yl)amino, (3-oxo-3,4-dihydro-2H-1,4-benzoxazin-6-yl)amino, (2-oxo-2,3-dihydro-1,3-benzoxazol-6-yl)amino, 3-oxo-1,3-dihydro-2-benzofuran-5-yl)amino, [2-(ethylsulphonyl)-1,3-benzothiazol-6-yl]amino, (2-methyl-1,3-benzothiazol-5-yl)amino, (1-acetyl-2,3-dihydro-1H-indol-5-yl)amino, (2,2,3,3-tetrafluoro-2,3-dihydro-1,4-benzodioxin-6-yl)amino, (2,2-dioxido-1,3-dihydro-2-benzothiophen-5-yl)amino, (2-oxo-2,3-dihydro-1H-indol-6-yl)amino, (2-oxo-1,2,3,4-tetrahydroquinolin-7-yl)amino, 1H-indazol-6-ylamino,
R⁶ represents hydrogen, C₁-C₃-alkyl, CH₂OCH₃, CH₂CH₂OCH₃, formyl, (C₁-C₄-alkyl)carbonyl, (C₁-C₄-haloalkyl)carbonyl, (C₁-C₄-alkoxy)carbonyl, benzyloxycarbonyl, benzyl, 4-methoxybenzyl, 2,4-dimethoxybenzyl, 2-hydroxybenzyl, unsubstituted or substituted benzoyl, unsubstituted or substituted C₂-C₄-alkenyl, unsubstituted or substituted C₂-C₄-alkynyl, C₁-C₂-alkylsulphinyl or C₁-C₂-alkylsulphonyl,
   where the substituents independently of one another are selected from the group consisting of hydrogen, fluorine, chlorine and bromine, C₁-C₂-alkyl, C₁-C₂-haloalkyl and cyano,
R⁷ represents hydrogen, methyl, cyano, CF₃, CFH₂ or CF₂H,
R⁸ represents chlorine, bromine, iodine, cyano, Me, SMe, SOMe, SO₂Me, CFH₂, CF₂H, or CF₃,
R⁹ represents hydrogen, methyl, ethyl, propan-1-yl, CH₂CH₂OMe or CH₂CH═CH₂,
R¹⁰ represents a group of the formula E1

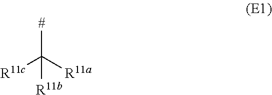

in which the symbols are as defined below and # represents the point of attachment to the nitrogen atom:
R^{11a} represents hydrogen or methyl,
R^{11b} represents hydrogen or C₁-C₃-alkyl,
R^{11c} represents hydrogen, C₁-C₄-alkyl, CN, CH₂CN, CH(Me)CN, C(Me)₂CN, CH₂OH, CH₂O—(C₁-C₃-alkyl), CH₂O—(C₁-C₃-haloalkyl), C(Me)HOH, C(Me)₂OH, C(Me)HO—(C₁-C₃-alkyl), C(Me)₂O—(C₁-C₃-alkyl), C(Me)HO—(C₁-C₂-haloalkyl), C(Me)₂O—(C₁-C₂-haloalkyl), CH₂S—(C₁-C₃-alkyl), C(Me)HS—(C₁-C₃-alkyl), C(Me)₂S—(C₁-C₃-alkyl), Si(C₁-C₂-alkyl)₃ or C₂-C₄-alkenyl which is optionally substituted by halogen or CF₃;
   or
R¹⁰ represents a cyclopentyl or a cyclohexyl ring which may optionally be mono- or polysubstituted by CN, halogen, C₁-C₃-alkyl, C₁-C₃-alkoxy, C₁-C₃-haloalkyl, C₁-C₃-haloalkoxy or C₁-C₃-alkoxy-C₁-C₃-alkyl,
   and also fungicidally active salts of these compounds.

Particular preference is given to using, as fungicides, compounds of the formula (I) in which one or more of the symbols have one of the meanings below:
X¹ represents nitrogen or CR³,
X² represents nitrogen or CR⁴,
   where X¹ and X² do not both represent nitrogen,
R¹ and R⁵ independently of one another represent hydrogen, C₁-C₂-alkyl, C₁-C₂-alkoxy, F or Cl,
R² represents hydrogen, fluorine, chlorine, bromine, iodine, nitro, cyano, C₁-C₃-alkyl, C₁-C₂-haloalkyl, hydroxyl, C₁-C₃-alkoxy, OCH₂CH═CH₂, OCH₂C≡CH, OCONHMe, C₁-C₂-haloalkoxy, COMe, COH, COEt, CMe(═NOH), CMe(═NOMe), SF₅, S—(C₁-C₃)-alkyl, SCF₃, SCF₂H, SOMe, SO₂Me, CO₂—(C₁-C₃-alkyl), NH₂, NH—(C₁-C₂-alkyl), N—(C₁-C₂-alkyl)₂, NHCO—(C₁-C₃-alkyl), NHCOH, NMeCO—(C₁-C₃-alkyl), NHCO₂—(C₁-C₃-alkyl),
R³ and R⁴ independently of one another represent hydrogen, fluorine, chlorine, bromine, iodine, cyano, nitro, hydroxyl, O—(C₁-C₄-alkyl), O-cyclopentyl, OCF₃, OCF₂H, OCF₂CF₃, OCF₂CF₂H, OCONH—(C₁-C₃-alkyl), OCON—(C₁-C₃-alkyl)₂, OCO—(C₁-C₄-alkyl), SH, SF₅,

S—C₁-C₃-alkyl, SCF₃, SCF₂H, SPh, SOMe, SO₂Me, SO₂CH₂CH═CH₂, SO₂CH₂C≡CH, SO₂CF₃, SO₂NMe₂, formyl, CO—(C₁-C₄-alkyl), COCF₃, COCH₂CN, CONH—(C₁-C₄-alkyl), CON—(C₁-C₄-alkyl)₂, CONHCH₂CF₃, CONHCH₂CH—CH₂, CONHCH₂C═CH, CONHCH₂C(═CH₂)CH₃, CONHCH(CH₃)CH₂OCH₃, CONH(CH₂)₂OCH₃, CONHPh, CONH-cyclopropyl, piperidin-1-ylcarbonyl, morpholin-4-ylcarbonyl, (4-methylpiperazin-1-yl)carbonyl, COOH, (C₁-C₄-alkoxy)carbonyl, CO₂(CH₂)₂OCH₃, NHCO—(C₁-C₄-alkyl), NHCOCF₃, N(C₂H₅)COMe, NHCOCH═CH₂, NHCOPh, NHCOC(CH₃)₂CH₂F, NHCOC(CH₃)₂CH₂Cl, NHCO(C═CH₂)CH₃, NHCOCH₂OCH₃, NHCO(CH₂)₂OCH₃, N(CH₃)COCH₃, NHCHO, NMeCHO, NHCO₂—(C₁-C₄-alkyl), NHCO₂Ph, NHCO₂CH₂CH₂Cl, NMeCO₂Me, NH₂, NH—(C₁-C₄-alkyl), N—(C₁-C₂-alkyl)₂, cyclopropylamino, NHCH(CH₃)CH₂OCH₃, acetyl(cyclopropyl)amino, [(1-methylcyclopropyl)carbonyl]amino, morpholin-1-yl, morpholin-4-ylmethyl, NHSO₂Me, CH₂CN, CHMeCN, CH₂SO₂NH—(C₁-C₄-alkyl), CH₂SO₂N—(C₁-C₄-alkyl)₂, C(Me)HSO₂N—(C₁-C₄-alkyl)₂, CH₂COCH₃, CH₂COtertBu, CH(CH₃)COCH₃, CH₂COCH(CH₃)₂, CH₂CO-cyclopropyl, CH₂CONHtertBu, CH₂CO₂—(C₁-C₄-alkyl), CH₂NHCOOMe, CH═NOMe, C(CH₃)═NOMe, CH═NOEt, C(CH₃)═NOEt, CH₂NHAc, CH₂NHCOCF₃, C₁-C₄-alkyl, C₃-C₆-cycloalkyl, 1-methoxycyclopropyl, 1-chlorocyclopropyl, 3,3-dimethylbutyl, neopentyl, C₂-C₄-alkenyl, C₁-C₂-haloalkyl, morpholin-4-ylsulphonyl, 3-methyl-2,5-dioxoimidazolidin-1-yl, 3-methyl-2-oxoimidazolidin-1-yl, (3-methyl-2-oxoimidazolidin-1-yl)methyl, piperidin-1-ylsulphonyl, 1,3-thiazol-2-yl, 1,3-thiazol-4-yl, 3,5-dimethylpiperidin-1-yl, 4-(tert-butoxycarbonyl)piperazin-1-yl, 5-ethoxy-3,4-dimethyl-1H-pyrazol-1-yl, acetyl(cyclohexyl)amino, 2-furoylamino, (2,2,2-trifluoroethyl)carbamoyl, 5-ethoxy-3-(trifluoromethyl)-1H-pyrazol-1-yl, 3-(2-chloroethyl)-2-oxoimidazolidin-1-yl, 1,1-dioxidoisothiazolidin-2-yl, 1,1-dioxidotetrahydrothiophen-2-yl, 4-methyl-5-oxo-4,5-dihydro-1H-1,2,4-triazol-1-yl, 3-methyl-5-oxo-2,5-dihydro-1H-pyrazol-1-yl, 2-(methoxymethyl)pyrrolidin-1-yl, 2-oxocyclopentyl, 2-oxotetrahydrofuran-3-yl, 1-methyl-3-oxo-2,3-dihydro-1H-pyrazol-4-yl, 1-methyl-3-oxopyrazolidin-4-yl, tetrahydrofuran-2-yl, furan-2-yl, 5-methyl-1,1-dioxido-1,2,5-thiadiazolidin-2-yl, (2,2-dimethylpropanoyl)-(methyl)amino, (dimethylsulphamoyl)methyl, (benzylsulphamoyl)methyl, (furan-2-ylcarbonyl)amino, [(1-phenylethyl)sulphamoyl]methyl, 5-oxo-3-(propan-2-yl)-4,5-dihydro-1H-pyrazol-1-yl, 5-oxo-3-(trifluoromethyl)-4,5-dihydro-1H-pyrazol-1-yl, (cyclopropylcarbonyl)amino, 4,4-dimethyl-2,5-dioxoimidazolidin-1-yl, 3-(1-methylethyl)-2-oxoimidazolidin-1-yl, 3-(2-methylpropyl)-2-oxoimidazolidin-1-yl, 2-oxo-3-prop-2-en-1-ylimidazolidin-1-yl, 3-tert-butyl-2-oxoimidazolidin-1-yl, pyrrolidin-1-ylsulphonyl, 2,5-dioxoimidazolidin-4-yl, (morpholin-4-ylsulphonyl)methyl, (piperidin-1-ylsulphonyl)methyl, (pyrrolidin-1-ylsulphonyl)methyl, 2-oxoimidazolidin-1-yl, 3-methyl-5-oxo-4,5-dihydro-1H-pyrazol-1-yl, 3,4-dimethyl-5-oxo-4,5-dihydro-1H-pyrazol-1-yl, 1-methylcyclopentyl, pyrrolidin-1-yl, piperidin-1-yl, 2-oxo-2,5-dihydro-1H-pyrrol-1-yl, 3,3-dimethyl-2-oxocyclopentyl, 3-oxo-4,5-dimethyl-2,4-dihydropyrazol-2-yl, 3-oxo-4-ethyl-5-methyl-2,4-dihydropyrazol-2-yl, 3,5-dioxo-4-ethylpyrazolidin-1-yl, 3-oxo-4,4-dimethylpyrazolidin-1-yl, 2,5-dioxo-2,5-dihydro-1H-pyrrol-1-yl, 4-methoxy-2-oxo-2,5-dihydro-1H-pyrrol-1-yl, 2-oxo-2,5-dihydro-1H-pyrrol-1-yl, 5-oxo-4,5-dihydro-1H-imidazol-1-yl, 1,1-dioxido-1,2-thiazinan-2-yl, 6-methyl-1,1-dioxido-1,2,6-thiadiazinan-2-yl, cyclopropyl(trifluoroacetyl)amino,

and, if R³ and R⁴, optionally via R¹² or R¹³, form a cycle, the following subunit from the general formula (I):

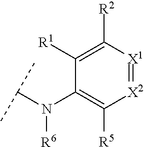

may be (2-oxo-2,3-dihydro-1H-indol-5-yl)amino, 1H-indol-6-ylamino, 1H-indol-5-ylamino, (4-methyl-3-oxo-3,4-dihydro-2H-1,4-benzoxazin-6-yl)amino, (4-methyl-3-oxo-3,4-dihydro-2H-1,4-benzoxazin-7-yl)amino, (1-acetyl-2,3-dihydro-1H-indol-6-yl)amino, (1-acetyl-2,3-dihydro-1H-indol-5-yl)amino, (2-oxo-2,3,4,5-tetrahydro-1H-1-benzazepin-8-yl)amino, 1,3-benzodioxol-5-ylamino, (1,3-dioxo-2,3-dihydro-1H-isoindol-5-yl)amino, (2-oxo-2,3-dihydro-1,3-benzoxazol-5-yl)amino, (2-ethyl-1,3-benzoxazol-5-yl)amino, (2-oxo-1,3-benzoxathiol-5-yl)amino, (2-oxo-1,2,3,4-tetrahydroquinolin-6-yl)amino, (3-oxo-3,4-dihydro-2H-1,4-benzoxazin-6-yl)amino, (2-oxo-2,3-dihydro-1,3-benzoxazol-6-yl)amino, [2-(ethylsulphonyl)-1,3-benzothiazol-6-yl]amino, (2-methyl-1,3-benzothiazol-5-yl)amino, (2,2,3,3-tetrafluoro-2,3-dihydro-1,4-benzodioxin-6-yl)amino, (3-oxo-1,3-dihydro-2-benzofur-5-yl)amino, (2,2-dioxido-1,3-dihydro-2-benzothiophen-5-yl) or (1-oxo-2,3-dihydro-1H-inden-5-yl)amino,
R⁶ represents hydrogen, Me, ethyl, propanyl, formyl, CH₂OCH₃, COMe, COCF₃, COOMe, COOEt, COOtertBu, benzyloxycarbonyl, benzyl, benzoyl or 2-methylbenzoyl,
R⁷ represents hydrogen, methyl or CF₃,
R⁸ represents chlorine, bromine, CF₃, Me, cyano, iodine, CFH₂, CF₂H, SMe, SOMe or SO₂Me,
R⁹ represents hydrogen, methyl, ethyl, propan-1-yl, CH₂CH₂OMe or CH₂CH═CH₂,
R¹⁰ represents a group of the formula E1

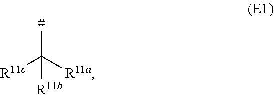

in which the symbols are as defined below and # represents the point of attachment to the nitrogen atom:
R^{11a} represents hydrogen or methyl,
R^{11b} represents hydrogen or C₁-C₃-alkyl,
R^{11c} represents hydrogen, C₁-C₄-alkyl, CN, CH₂CN, CH(Me)CN, CH₂OH, CH₂O—(C₁-C₂-alkyl), CH₂O—(C₁-C₂-haloalkyl), C(Me)HOH, C(Me)₂OH, C(Me)HO—(C₁-C₂-alkyl), C(Me)₂O—(C₁-C₂-alkyl), CH₂S—(C₁-C₂-alkyl), C(Me)HS—(C₁-C₂-alkyl), C(Me)S—(C₁-C₂-alkyl), SiMe₃ or C₂-C₄-alkenyl which is optionally substituted by halogen or CF₃;
   or
R¹⁰ represents a cyclopentyl or a cyclohexyl ring which may optionally be mono- or polysubstituted by CN, halogen, Me, Et, OMe, CF₃, OCF₃, CH₂OCH₃,
   and fungicidally active salts of these compounds.

Very particular preference is given to using, as fungicides, compounds of the formula (I, in which one or more of the symbols have one of the meanings below:
X¹ represents nitrogen or CR³,
X² represents nitrogen or CR⁴,
   where X¹ and X² do not both represent nitrogen,
R¹ and R⁵ independently of one another represent hydrogen, methyl, methoxy or F,
R² represents hydrogen, fluorine, chlorine, bromine, iodine, nitro, cyano, methyl, ethyl, propanyl, propan-2-yl, CF₃, CF₂H, CFH₂, CF₂CF₃, hydroxyl, OMe, OEt, OPr, OisoPr, OCH₂CH═CH₂, OCH₂C═CH, OCONHMe, OCF₃, OCF₂CF₃, OCF₂CF₂H, COMe, COH, COEt, C(═NOH)Me, C(═NOMe)Me, SF₅, SMe, SEt, SPr, SiPr, SCF₃, SCF₂H, SOMe, SO₂Me, CO₂CH₃, CO₂Et, NHMe, NMe₂, NHEt, NEt₂, NHCOMe, NHCOH, NMeCOMe, NHCO₂Me or NHCO₂Et,
R³ and R⁴ independently of one another represent hydrogen, fluorine, chlorine, bromine, iodine, cyano, nitro, hydroxyl, OMe, OEt, OPr, OisoPr, OBu, OsecBu, OisoBu, OtertBu, O-cylopentyl, OCF₃, OCF₂HOCF₂CF₃, OCF₂CF₂H, OCONHMe, OCONHEt, OCONHPr, OCONHisoPr, OCONMe₂, OCONEt₂, OCONPr₂, OCONisoPr₂, OCOMe, OCOEt, OCOBu, SH, SF₅, SMe, SEt, SPr, SiPr, SCF₃, SCF₂H, SPh, SOMe, SO₂Me, SO₂CH₂CH—CH₂, SO₂CH₂C—CH, SO₂CF₃, SO₂NMe₂, formyl, COMe, COEt, COPr, COisoPr, COCF₃, COCH₂CN, CONHMe, CONMe₂, CONHEt, CONHCH₂CF₃, CONEt₂, CONHPr, CONHisoPr, CONHBu, CONHtertBu, CONHCH₂CH═CH₂, CONHCH₂C≡CH, CONHCH₂C(═CH₂)CH₃, CONHCH(CH₃)CH₂OCH₃, CONH(CH₂)₂OCH₃, CONHPh, CONH-cyclopropyl, piperidin-1-ylcarbonyl, morpholin-4-ylcarbonyl, (4-methylpiperazin-1-yl)carbonyl, COOH, CO₂CH₃, CO₂Et, CO₂Pr, CO₂ isoPr, CO₂(CH₂)₂OCH₃, NHCOMe, NHCOEt, NHCOPr, NHCOisoPr, NHCOBu, NHCOisoBu, NHCOCF₃, NHCOsecBu, NHCOtertBu, N(C₂H₅)COMe, NHCOCH═CH₂, NHCOPh, NHCOC(CH₃)₂CH₂F, NHCOC(CH₃)₂CH₂Cl, NHCO(C—CH₂)CH₃, NHCOCH₂OCH₃, NHCO(CH₂)₂OCH₃, N(CH₃)COCH₃, NHCHO, NMeCHO, NHCO₂Me, NHCO₂Et, NHCO₂ isoPr, NHCO₂tertBu, NHCO₂ isoBu, NHCO₂secBu, NHCO₂nBu, NHCO₂Ph, NHCO₂CH₂CH₂Cl, NMeCO₂Me, NH₂, NHMe, NMe₂, NHEt, NEt₂, NHPr, NHisoPr, NHnBu, cyclopropylamino, NHCH(CH₃)CH₂OCH₃, acetyl(cyclopropyl)amino, [(1-methylcyclopropyl)carbonyl]amino, morpholin-1-yl, morpholin-4-yl-methyl, NHSO₂Me, CH₂CN, CHMeCN, CH₂SO₂NHMe, CH₂SO₂NHPr, CH₂COCH₃, CH₂COtertBu, CH(CH₃)COCH₃, CH₂COCH(CH₃)₂, CH₂CO-cyclopropyl, CH₂CONHtertBu, CH₂CO₂Et, CH₂CO₂Me, CH₂NHCOOMe, CH═NOMe, C(CH₃)═NOMe, CH═NOEt, C(CH₃)═NOEt, CH₂NHAc, CH₂NHCOCF₃, methyl, ethyl, propyl, 1-methylethyl, butyl, 1-methylpropyl, 2-methylpropyl, 1,1-dimethylethyl, cyclopropyl, 1-methoxycyclopropyl, 1-chlorocyclopropyl, cyclobutyl, 3,3-dimethylbutyl, cyclopentyl, cyclohexyl, neopentyl, prop-2-en-1-yl, 1-methylprop-2-en-1-yl, but-3-en-1-yl, CF₃, CF₂H, CF₂CF₃, morpholin-4-ylsulphonyl, 3-methyl-2,5-dioxoimidazolidin-1-yl, 3-methyl-2-oxoimidazolidin-1-yl, (3-methyl-2-oxoimidazolidin-1-yl)methyl, piperidin-1-ylsulphonyl, 1,3-thiazol-2-yl, 1,3-thiazol-4-yl, 3,5-dimethylpiperidin-1-yl, 4-(tert-butoxycarbonyl)piperazin-1-yl, 5-ethoxy-3,4-dimethyl-1H-pyrazol-1-yl, acetyl(cyclo-hexyl)amino, 2-furoylamino, (2,2,2-trifluoroethyl)carbamoyl, 5-ethoxy-3-(trifluoromethyl)-1H-pyrazol-1-yl, 3-(2-chloroethyl)-2-oxoimidazolidin-1-yl, 1,1-dioxidoisothiazolidin-2-yl, 1,1-dioxidotetrahydrothiophen-2-yl, 4-methyl-5-oxo-4,5-dihydro-1H-1,2,4-triazol-1-yl, 3-methyl-5-oxo-2,5-dihydro-1H-pyrazol-1-yl, 2-(methoxy-methyl)pyrrolidin-1-yl, 2-oxocyclopentyl, 2-oxotetrahydrofuran-3-yl, 1-methyl-3-oxo-2,3-dihydro-1H-pyrazol-4-yl, 1-methyl-3-oxopyrazolidin-4-yl, tetrahydrofuran-2-yl, furan-2-yl, 5-methyl-1,1-dioxido-1,2,5-thiadiazolidin-2-yl, (2,2-dimethylpropanoyl)(methyl)amino, (benzylsulphamoyl)methyl, (dimethylsulphamoyl)methyl, (furan-2-ylcarbonyl)amino, [(1-phenylethyl)sulphamoyl]methyl, [ethyl(methyl)sulphamoyl]methyl, [methyl(propan-2-yl)sulphamoyl]methyl, 5-oxo-3-(propan-2-yl)-4,5-dihydro-1H-pyrazol-1-yl, 5-oxo-3-(trifluoromethyl)-4,5-dihydro-1H-pyrazol-1-yl, (cyclopropylcarbonyl)amino, 3-(1-methylethyl)-2-oxoimidazolidin-1-yl, 3-(2-methylpropyl)-2-oxoimidazolidin-1-yl, 2-oxo-3-prop-2-en-1-ylimidazolidin-1-yl, 3-tert-butyl-2-oxoimidazolidin-1-yl, pyrrolidin-1-ylsulphonyl, 2,5-dioxoimidazolidin-4-yl, (morpholin-4-ylsulphonyl)methyl, (piperidin-1-ylsulphonyl)methyl, (pyrrolidin-1-ylsulphonyl)methyl, 2-oxoimidazolidin-1-yl, 3,3-dimethyl-2-oxocyclopentyl, 3-oxo-4,5-dimethyl-2,4-dihydropyrazol-2-yl, 3,5-dioxo-4-ethylpyrazolidin-1-yl, 5-oxo-4,5-dihydro-1H-imidazol-1-yl, 1,1-dioxido-1,2-thiazinan-2-yl, 6-methyl-1,1-dioxido-1,2,6-thiadiazinan-2-yl, cyclopropyl(trifluoracetyl)amino,
   and, if R³ and R⁴, if appropriate via R¹² or R¹³, form a cycle, the subunit below of the general formula (I):

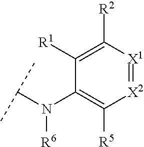

may be (2-oxo-2,3-dihydro-1H-indol-5-yl)amino, 1H-indol-6-ylamino, 1H-indol-5-ylamino, (4-methyl-3-oxo-3,4-dihydro-2H-1,4-benzoxazin-6-yl)amino, (4-methyl-3-oxo-3,4-dihydro-2H-1,4-benzoxazin-7-yl)amino, (1-acetyl-2,3-dihydro-1H-indol-6-yl)amino, (1-acetyl-2,3-dihydro-1H-indol-5-yl)amino, (2-oxo-2,3,4,5-tetrahydro-1H-1-benzazepin-8-yl)amino, 1,3-benzodioxol-5-ylamino, (1,3-dioxo-2,3-dihydro-1H-isoindol-5-yl)amino, (2-oxo-2,3-dihydro-1,3-benzoxazol-5-yl)amino, (2-ethyl-1,3-benzoxazol-5-yl)amino, (2-oxo-1,3-benzoxathiol-5-yl)amino, (2-oxo-1,2,3,4-tetrahydroquinolin-6-yl)amino, (3-oxo-3,4-dihydro-2H-1,4-benzoxazin-6-yl)amino, (2-oxo-2,3-dihydro-1,3-benzoxazol-6-yl)amino, [2-(ethylsulphonyl)-1,3-benzothiazol-6-yl]amino, (2-methyl-1,3-benzothiazol-5-yl)amino, (2,2,3,3-tetrafluoro-2,3-dihydro-1,4-benzodioxin-6-yl)amino, (3-oxo-1,3-dihydro-2-benzofur-5-yl)amino, (2,2-dioxido-1,3-dihydro-2-benzothiophen-5-yl) or (1-oxo-2,3-dihydro-1H-inden-5-yl)amino,
R⁶ represents hydrogen, Me, formyl, COMe, COCF₃, COOMe or COOEt,
R⁷ represents hydrogen,
R⁸ represents chlorine, bromine, CF₃, Me, cyano, iodine, CFH₂, SMe, SOMe or SO₂Me,
R⁹ represents hydrogen, methyl, ethyl, propan-1-yl, CH₂CH₂OMe or CH₂CH═CH₂,
R¹⁰ represents methyl, ethyl, propanyl, propan-2-yl, butanyl, butan-2-yl, 2-methylpropanyl, tert-butyl, pentanyl, 3-methylbutanyl, 2-methylbutanyl, 2,2-dimethylpropanyl, pentan-2-yl, pentan-3-yl, 4-methylpentan-2-yl, hexan-3-yl, 3,3-dimethylbutan-2-yl, 2,4-dimethylpentan-3-yl, cyclopentyl, 2-methylcyclopentyl, 2-ethylcyclopentyl, 2-chlorocyclopentyl, 2-fluorocyclopentyl, 2-cyanocyclopentyl, 2-methoxycyclopentyl, 2-(methoxymethyl)-cyclopentyl, cyclohexyl, 2-methoxycyclohexyl, 2-methylcyclohexyl, 4,4-difluoro-cyclohexyl, 4-(trifluoromethyl)cyclohexyl, prop-2-en-1-yl, 2-methylprop-2-en-1-yl, 1-methylprop-2-en-1-yl, 3-methylbut-2-en-1-yl, but-2-en-1-yl, 4,4,4-trifluorobut-2-en-1-yl, 3-chlorobut-2-en-1-yl, 2-chloroprop-2-en-1-yl, cyanomethyl, 2-cyanoethyl, 1-cyano-propan-2-yl, 3-cyanobutan-2-yl, (trimethylsilyl)methyl, 1-(trimethylsilyl)ethyl, 2-hydroxyethyl, 2-methoxyethyl, 2-ethoxyethyl, 2-(trifluoromethoxy)ethyl, 1-hydroxypropan-2-yl, 1-hydroxybutan-2-yl, 3-hydroxy-3-methylbutan-2-yl, 2-hydroxy-2-methylpropanyl, 3-methoxy-3-methylbutan-2-yl, 2-methoxy-2-methylpropanyl, 1-methoxypropan-2-yl, 1-ethoxypropan-2-yl, 1-trifluoromethoxypropan-2-yl, 1-methoxybutan-2-yl, 3-methoxybutan-2-yl, 1-methoxy-3-methylbutan-2-yl, 1-methoxypentan-2-yl, 2-(methylsulphanyl)ethyl, (2R)-1-(methylsulphanyl)propan-2-yl, (2S)-1-(methylsulphanyl)propan-2-yl, 2-methyl-1-(methylsulphanyl)propan-2-yl,
   and the fungicidally active salts of these compounds.

Special preference is given to compounds of the formula (I) in which one or more of the symbols have one of the meanings below:
X¹ represents CR³,
X² represents nitrogen or CR⁴,
R¹ and R⁵ independently of one another represent hydrogen, methyl, methoxy or F,
R² represents hydrogen, fluorine, chlorine, bromine, cyano, methyl, ethyl, propanyl, propan-2-yl, CF₃, hydroxyl, OMe, OEt, OCF₃, COMe, COEt, SMe, SEt, CO₂CH₃, CO₂Et, NHMe, NMe₂, NHEt, NEt₂ or NHCOMe,
R³ and R⁴ independently of one another represent hydrogen, fluorine, chlorine, bromine, iodine, cyano, nitro, hydroxyl, OMe, OEt, OPr, OisoPr, OBu, OsecBu, OisoBu, OtertBu, O-cylopentyl, OCF₃, OCF₂CF₃, OCF₂CF₂H, OCONHMe, OCONHEt, OCONHPr, OCONHisoPr, OCONMe₂, OCONEt₂, OCONPr₂, OCONisoPr₂, OCOMe, OCOEt, OCOBu, SH, SF₅, SMe, SEt, SPr, SiPr, SCF₃, SCF₂H, SPh, SO₂Me, SO₂CF₃, SO₂NMe₂, formyl, COMe, COEt, COPr, COisoPr, COCF₃, COCH₂CN, CONHMe, CONMe₂, CONHEt, CONHCH₂CF₃, CONEt₂, CONHPr, CONHisoPr, CONHBu, CONHtertBu, CONHCH₂CH═CH₂, CONHCH₂C═CH, CONHCH₂C(═CH₂)CH₃, CONHCH(CH₃)CH₂OCH₃, CONH(CH₂)₂OCH₃, CONHPh, CONH-cyclopropyl, piperidin-1-ylcarbonyl, morpholin-4-ylcarbonyl, COOH, CO₂CH₃, CO₂Et, CO₂Pr, CO₂isoPr, CO₂(CH₂)₂OCH₃, NHCOMe, NHCOEt, NHCOPr, NHCOisoPr, NHCOBu, NHCOisoBu, NHCOCF₃, NHCOsecBu, NHCOtertBu, N(C₂H₅)COMe, NHCOCH—CH₂, NHCOPh, NHCOC(CH₃)₂CH₂F, NHCOC(CH₃)₂CH₂Cl, NHCO(C—CH₂)CH₃, NHCOCH₂OCH₃, NHCO(CH₂)₂OCH₃, N(CH₃)COCH₃, NHCHO, NMeCHO, NHCO₂Me, NHCO₂Et, NHCO₂ isoPr, NHCO₂tertBu, NHCO₂ isoBu, NHCO₂secBu, NHCO₂nBu, NHCO₂Ph, NHCO₂CH₂CH₂Cl, NMeCO₂Me, NH₂, NHMe, NMe₂, NHEt, NEt₂, NHPr, NHisoPr, NHnBu, cyclopropylamino, NHCH(CH₃)CH₂OCH₃, acetyl(cyclopropyl)amino, [(1-methyl-cyclopropyl)carbonyl]amino, morpholin-1-yl, morpholin-4-ylmethyl, NHSO₂Me, CH₂CN, CHMeCN, CH₂SO₂NHMe, CH₂SO₂NHPr, CH₂COCH₃, CH₂COtertBu, CH(CH₃)COCH₃, CH₂COCH(CH₃)₂, CH₂CO-cyclopropyl, CH₂CONHtertBu, CH₂CO₂Et, CH₂CO₂Me, CH₂NHCOOMe, CH₂NHAc, CH₂NHCOCF₃, methyl, ethyl, propyl, 1-methylethyl, butyl, 1-methylpropyl, 2-methylpropyl, 1,1-dimethylethyl, cyclopropyl, 1-methoxycyclopropyl, 1-chlorocyclopropyl, cyclobutyl, 3-dimethylbutyl, cyclopentyl, cyclohexyl, neopentyl, prop-2-en-1-yl, 1-methylprop-2-en-1-yl, but-3-en-1-yl, CF₃, CF₂H, morpholin-4-ylsulphonyl, 3-methyl-2,5-dioxoimidazolidin-1-yl, 3-methyl-2-oxoimidazolidin-1-yl, (3-methyl-2-oxoimidazolidin-1-yl)methyl, 1,3-thiazol-2-yl, 1,3-thiazol-4-yl, 3,5-dimethylpiperidin-1-yl, 4-(tert-butoxycarbonyl)piperazin-1-yl, acetyl(cyclohexyl)amino, 2-furoylamino, 3-methyl-5-oxo-2,5-dihydro-1H-pyrazol-1-yl, tetrahydrofuran-2-yl, furan-2-yl, 5-methyl-1,1-dioxido-1,2,5-thiadiazolidin-2-yl, (2,2-dimethylpropanoyl)(methyl)amino, (benzylsulphamoyl)methyl, (dimethylsulphamoyl)-methyl, (furan-2-ylcarbonyl)amino, [(1-phenylethyl)sulphamoyl]methyl, [ethyl(methyl)sulphamoyl]methyl, [methyl(propan-2-yl)sulphamoyl]methyl, 5-oxo-3-(propan-2-yl)-4,5-dihydro-1H-pyrazol-1-yl, 5-oxo-3-(trifluoromethyl)-4,5-dihydro-1H-pyrazol-1-yl, (cyclopropylcarbonyl)amino, 3-(1-methylethyl)-2-oxoimidazolidin-1-yl, 3-(2-methylpropyl)-2-oxoimidazolidin-1-yl, 2-oxo-3-prop-2-en-1-ylimidazolidin-1-yl, 3-tert-butyl-2-oxoimidazolidin-1-yl, pyrrolidin-1-ylsulphonyl, 2,5-dioxoimidazolidin-4-yl, (morpholin-4-ylsulphonyl)methyl, (piperidin-1-ylsulphonyl)methyl, (pyrrolidin-1-ylsulphonyl)methyl, 2-oxoimidazolidin-1-yl, 3,3-dimethyl-2-oxocyclopentyl, 3-oxo-4,5-dimethyl-2,4-dihydropyrazol-2-yl, 3,5-dioxo-4-ethylpyrazolidin-1-yl, 5-oxo-4,5-dihydro-1H-imidazol-1-yl, 1,1-dioxido-1,2-thiazinan-2-yl, 6-methyl-1,1-dioxido-1,2,6-thiadiazinan-2-yl, cyclopropyl(trifluoroacetyl)amino,
   and, if R³ and R⁴, if appropriate via R¹² or R¹³, form a cycle, the following subunit from the general formula (I):

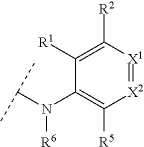

may represent (2-oxo-2,3-dihydro-1H-indol-5-yl)amino, 1H-indol-6-ylamino, 1H-indol-5-ylamino, (4-methyl-3-oxo-3,4-dihydro-2H-1,4-benzoxazin-6-yl)amino, (4-methyl-3-oxo-3,4-dihydro-2H-1,4-benzoxazin-7-yl)amino, (1-acetyl-2,3-dihydro-1H-indol-6-yl)amino, (1-acetyl-2,3-dihydro-1H-indol-5-yl)amino, (2-oxo-2,3,4,5-tetrahydro-1H-1-benzazepin-8-yl)amino, 1,3-benzodioxol-5-ylamino, (2-oxo-1,3-benzoxathiol-5-yl)amino, (2-oxo-1,2,3,4-tetrahydroquinolin-6-yl)amino, (3-oxo-3,4-dihydro-2H-1,4-benzoxazin-6-yl)amino, (2-oxo-2,3-dihydro-1,3-benzoxazol-6-yl)amino, (2-methyl-1,3-benzothiazol-5-yl)amino, (2,2,3,3-tetrafluoro-2,3-dihydro-1,4-benzodioxin-6-yl)amino, (3-oxo-1,3-dihydro-2-benzofur-5-yl)amino, 1-oxo-2,3-dihydro-1H-inden-5-yl)amino,
R⁶ represents hydrogen, Me, formyl or COMe,
R⁷ represents hydrogen,
R⁸ represents chlorine, bromine, CF₃, Me, cyano, iodine or CFH₂,
R⁹ represents hydrogen, methyl, ethyl, propan-1-yl, CH₂CH₂OMe or CH₂CH═CH₂,
R¹⁰ represents methyl, ethyl, propanyl, propan-2-yl, butanyl, butan-2-yl, 2-methylpropanyl, tert-butyl, pentanyl, 3-methylbutanyl, 2-methylbutanyl, 2,2-dimethylpropanyl, pentan-2-yl, pentan-3-yl, 4-methylpentan-2-yl, hexan-3-yl, 3,3-dimethylbutan-2-yl, 2,4-dimethylpentan-3-yl, cyclopentyl, 2-methylcyclopentyl, 2-ethylcyclopentyl, 2-chlorocyclopentyl, 2-fluoro-cyclopentyl, 2-cyanocyclopentyl, 2-methoxycyclopentyl, 2-(methoxymethyl)cyclopentyl, cyclohexyl, 2-methoxycyclohexyl, 2-methylcyclohexyl, 4,4-difluorocyclohexyl, 4-(trifluoromethyl)cyclohexyl, prop-2-en-1-yl, 2-methylprop-2-en-1-yl, 1-methylprop-2-en-1-yl, 3-methylbut-2-en-1-yl, but-2-en-1-yl, 4,4,4-trifluorobut-2-en-1-yl, 3-chlorobut-2-en-1-yl, 2-chloroprop-2-en-1-yl, cyanomethyl, 2-cyanoethyl, 1-cyanopropan-2-yl, 3-cyanobutan-2-yl, (trimethylsilyl)methyl, 1-(trimethylsilyl)ethyl, 2-hydroxyethyl, 2-methoxyethyl, 2-ethoxyethyl, 2-(trifluoromethoxy)ethyl, 1-hydroxypropan-2-yl, 1-hydroxybutan-2-yl, 3-hydroxy-3-methylbutan-2-yl, 2-hydroxy-2-methylpropanyl, 3-methoxy-3-methylbutan-2-yl, 2-methoxy-2-methylpropanyl, 1-methoxypropan-2-yl, 1-ethoxypropan-2-yl, 1-trifluoromethoxypropan-2-yl, 1-methoxybutan-2-yl, 3-methoxybutan-2-yl, 1-methoxy-3-methylbutan-2-yl, 1-methoxypentan-2-yl, 2-(methylsulphanyl)ethyl, (2R)-1-(methylsulphanyl)propan-2-yl, (2S)-1-(methylsulphanyl)propan-2-yl, 2-methyl-1-(methylsulphanyl)propan-2-yl,
   and agrochemically active salts thereof.

Special preference is furthermore given to using, as fungicides, compounds of the formula (I) in which one or more of the symbols have one of the meanings below:
X¹ represents CR³,
X² represents nitrogen or CR⁴,
R¹ and R⁵ independently of one another represent hydrogen, methyl, methoxy or F,
R² represents hydrogen, fluorine, chlorine, bromine, cyano, methyl, CF₃, hydroxyl, OMe, OEt, OCF₃, COMe, SMe, CO₂CH₃, CO₂Et, NHMe, NMe₂, NHEt, NEt₂ or NHCOMe,
R³ and R⁴ independently of one another represent hydrogen, fluorine, chlorine, bromine, cyano, hydroxyl, OMe, OEt, OPr, OisoPr, OCF₃, OCF₂CF₃, OCF₂CF₂H, OCONHMe, OCONHEt, OCONHisoPr, OCONMe₂, SF₅, SMe, SEt, SPr, SiPr, SCF₃, SCF₂H, SO₂Me, SO₂CF₃, SO₂NMe₂, formyl, COMe, COEt, COCF₃, COCH₂CN, CONHMe, CONMe₂, CONHEt, CONEt₂, CONHPr, CONHisoPr, CONHBu, CONHtertBu, CONHCH₂CH═CH₂, CONHCH₂C≡CH, CONHPh, CONH-cyclopropyl, piperidin-1-ylcarbonyl, morpholin-4-ylcarbonyl, CO₂CH₃, CO₂Et, CO₂Pr, CO₂ isoPr, CO₂(CH₂)₂OCH₃, NHCOMe, NHCOEt, NHCOPr, NHCOisoPr, NHCOCH₃, NHCOtertBu, N(C₂H₅)COMe, NHCOCH═CH₂, NHCOPh, NHCOC(CH₃)₂CH₂F, NHCO(C═CH₂)CH₃, N(CH₃)COCH₃, NHCHO, NMeCHO, NHCO₂Me, NHCO₂Et, NHCO₂Ph, NHMe, NMe₂, NHEt, NEt₂, NHPr, NHisoPr, cyclopropylamino, acetyl(cyclopropyl)amino, [(1-methylcyclopropyl)-carbonyl]amino, morpholin-1-yl, CH₂CN, CHMeCN, CH₂SO₂NHMe, CH₂SO₂NHPr, CH₂COCH₃, CH₂COtertBu, CH₂CO₂Et, CH₂CO₂Me, CH₂NHAc, methyl, ethyl, propyl, 1-methylethyl, butyl, 1-methylpropyl, 2-methylpropyl, 1,1-dimethylethyl, prop-2-en-1-yl, CF₃, CF₂H, morpholin-4-ylsulphonyl, 3-methyl-2,5-dioxoimidazolidin-1-yl, 3-methyl-2-oxoimidazolidin-1-yl, (3-methyl-2-oxoimidazolidin-1-yl)methyl, piperidin-1-ylsulphonyl, 1,3-thiazol-2-yl, 1,3-thiazol-4-yl, 3,5-dimethylpiperidin-1-yl, 4-(tert-butoxycarbonyl)-piperazin-1-yl, acetyl(cyclohexyl)amino, 2-furoylamino, 3-methyl-5-oxo-2,5-dihydro-1H-pyrazol-1-yl, tetrahydrofuran-2-yl, furan-2-yl, 5-methyl-1,1-dioxido-1,2,5-thiadiazolidin-2-yl, (2,2-dimethylpropanoyl)(methyl)amino, (benzylsulphamoyl)methyl, (dimethylsulphamoyl)methyl, (furan-2-ylcarbonyl)amino, [(1-phenylethyl)sulphamoyl]methyl, [ethyl(methyl)sulphamoyl)methyl, [methyl(propan-2-yl)sulphamoyl]methyl, 5-oxo-3-(propan-2-yl)-4,5-dihydro-1H-pyrazol-1-yl, 5-oxo-3-(trifluoromethyl)-4,5-dihydro-1H-pyrazol-1-yl, cyclopropyl(trifluoroacetyl)amino,
   and, if R³ and R⁴, if appropriate via R¹² or R¹³, form a cycle, the subunit below from the general formula (I):

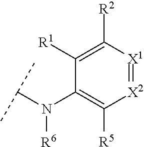

may represent (2-oxo-2,3-dihydro-1H-indol-5-yl)amino, 1H-indol-6-ylamino, 1H-indol-5-ylamino, (4-methyl-3-oxo-3,4-dihydro-2H-1,4-benzoxazin-6-yl)amino, (4-methyl-3-oxo-3,4-dihydro-2H-1,4-benzoxazin-7-yl)amino, (1-acetyl-2,3-dihydro-1H-indol-6-yl)amino, (1-acetyl-2,3-dihydro-1H-indol-5-yl)amino, (2-oxo-2,3,4,5-tetrahydro-1H-1-benzazepin-8-yl)amino, 1,3-benzodioxol-5-ylamino, (2-oxo-1,3-benzoxathiol-5-yl)amino, (2-oxo-1,2,3,4-tetrahydroquinolin-6-yl)amino, (3-oxo-3,4-dihydro-2H-1,4-benzoxazin-6-yl)amino, (2-oxo-2,3-dihydro-1,3-benzoxazol-6-yl)amino, (2-methyl-1,3-benzothiazol-5-yl)amino, (2,2,3,3-tetrafluoro-2,3-dihydro-1,4-benzodioxin-6-yl)amino, (3-oxo-1,3-dihydro-2-benzofur-5-yl)amino, 1-oxo-2,3-dihydro-1H-inden-5-yl)amino,
R⁶ represents hydrogen, Me, formyl or COMe,
R⁷ represents hydrogen,
R⁸ represents chlorine, bromine, CF₃, cyano, iodine or CFH₂,
R⁹ represents hydrogen, methyl, ethyl, propan-1-yl or CH₂CH═CH₂,
R¹⁰ represents methyl, ethyl, propanyl, propan-2-yl, butanyl, butan-2-yl, 2-methylpropanyl, tert-butyl, pentanyl, 3-methylbutanyl, 2-methylbutanyl, 2,2-dimethylpropanyl, pentan-2-yl, pentan-3-yl, 4-methylpentan-2-yl, hexan-3-yl, cyclopentyl, 2-methylcyclopentyl, 2-ethylcyclopentyl, 2-(methoxymethyl)cyclopentyl, cyclohexyl, (3-trifluoro-methyl)cyclohexyl, prop-2-en-1-yl, 2-methylprop-2-en-1-yl, 3-methylbut-2-en-1-yl, 2-chloroprop-2-en-1-yl, cyanomethyl, 2-cyanoethyl, (trimethylsilyl)methyl, 1-(trimethylsilyl)ethyl, 2-hydroxyethyl, 2-methoxyethyl, 2-ethoxyethyl, 2-(trifluoromethoxy)ethyl, 1-hydroxypropan-2-yl, 1-hydroxybutan-2-yl, 1-methoxypropan-2-yl, 1-ethoxypropan-2-yl, 1-trifluoromethoxypropan-2-yl, 1-methoxybutan-2-yl, 3-methoxybutan-2-yl, 1-methoxy-3-methylbutan-2-yl, 1-methoxypentan-2-yl, 2-(methylsulphanyl)ethyl, (2R)-1-(methylsulphanyl)propan-2-yl, (2S)-1-(methylsulphanyl)propan-2-yl, 2-methyl-1-(methylsulphanyl)propan-2-yl,
   and agrochemically active salts thereof.

Special preference is furthermore given to using, as fungicides, compounds of the formula (I) in which one or more of the symbols have one of the meanings below:
X¹ represents CR³,
X² represents nitrogen or CR⁴,
R¹ and R⁵ independently of one another represent hydrogen or F,
R² represents hydrogen, chlorine or OMe,
R³ represents hydrogen, fluorine, chlorine, cyano, hydroxyl, OMe, OisoPr, OCF₃, OCF₂CF₂H, SMe, SCF₃, SCF₂H, SO₂Me, SO₂NMe₂, formyl, COMe, COEt, COCH₂CN, CONHMe, CONMe₂, CONHtertBu, CONHCH₂CH═CH₂, CONHPh, CONH-cyclopropyl, CO₂CH₃, NHCOMe, NHCOisoPr, NHCOCF₃, NHCOtertBu, N(C₂H₅)COMe, N(CH₃)COCH₃, NHCHO, NHCO₂Me, NHCO₂Et, NEt₂, cyclopropylamino, [(1-methylcyclopropyl)carbonyl]amino, CH₂COCH₃, CH₂COtertBu, CH₂CO₂Me, methyl, ethyl, 1-methylethyl, CF₃, morpholin-4-ylsulphonyl, 3-methyl-2,5-dioxoimidazolidin-1-yl, 1,3-thiazol-4-yl, 4-(tert-butoxycarbonyl)piperazin-1-yl, acetyl(cyclohexyl)amino, (2,2-dimethylpropanoyl)(methyl)amino or cyclopropyl(trifluoroacetyl)amino,
R⁴ represents hydrogen, fluorine, chlorine, cyano, hydroxyl, OMe, OEt, OCF₃, OCF₂CF₂H, OCONHMe, SMe, SO₂NMe₂, formyl, COMe, COEt, COCH₂CN, CONHMe, CONMe₂, CONHtertBu, NHCOMe, NHCOisoPr, NHCOtertBu, NHCOC(CH₃)₂CH₂F, NHCO(C═CH₂)CH₃, N(CH₃)COCH₃, NHCHO, NHCO₂Me, NHCO₂Et, NHCO₂Ph, [(1-methylcyclopropyl)carbonyl]amino, CH₂CN, CH₂COCH₃, CH₂COtertBu, CH₂CO₂Me, methyl, ethyl, 1-methylethyl, CF₃, morpholin-4-ylsulphonyl, (3-methyl-2-oxoimidazolidin-1-yl)methyl, piperidin-1-ylsulphonyl, (benzylsulphamoyl)methyl, (dimethylsulphamoyl)methyl, (furan-2-ylcarbonyl)amino, [(1-phenylethyl)sulphamoyl]methyl, [ethyl-(methyl)sulphamoyl)methyl or [methyl(propan-2-yl)sulphamoyl]methyl,
   and, if R³ and R⁴ form a cycle, the subunit below from the general formula (I):

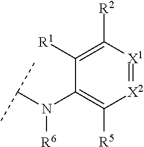

may be 1H-indol-6-ylamino, 1H-indol-5-ylamino, (4-methyl-3-oxo-3,4-dihydro-2H-1,4-benzoxazin-6-yl)amino, (1-acetyl-2,3-dihydro-1H-indol-6-yl)amino, (1-acetyl-2,3-dihydro-1H-indol-5-yl)amino, (2-oxo-2,3,4,5-tetrahydro-1H-1-benzazepin-8-yl)amino, 1,3-benzodioxol-5-ylamino, (2-oxo-1,3-benzoxathiol-5-yl)amino, (2-oxo-1,2,3,4-tetrahydroquinolin-6-yl)amino, (2-oxo-2,3-dihydro-1,3-benzoxazol-6-yl)amino, (2-methyl-1,3-benzothiazol-5-yl)amino, (2,2,3,3-tetrafluoro-2,3-dihydro-1,4-benzodioxin-6-yl)amino, (3-oxo-1,3-dihydro-2-benzofur-5-yl)amino or 1-oxo-2,3-dihydro-1H-inden-5-yl)amino,
R⁶ represents hydrogen,
R⁷ represents hydrogen,
R⁸ represents chlorine, bromine, CF₃ or cyano,
R⁹ represents hydrogen, methyl or ethyl,
R¹⁰ represents methyl, ethyl, propanyl, propan-2-yl, butanyl, butan-2-yl, tert-butyl, 4-methylpentan-2-yl, hexan-3-yl, cyclopentyl, cyclohexyl, prop-2-en-1-yl, cyanomethyl, 2-hydroxyethyl, 2-methoxyethyl, 2-ethoxyethyl, 2-(trifluoromethoxy)ethyl, 1-hydroxypropan-2-yl, 1-hydroxybutan-2-yl, 1-methoxypropan-2-yl, 1-methoxybutan-2-yl, 1-methoxy-3-methylbutan-2-yl, 1-methoxypentan-2-yl, 2-(methylsulphanyl)ethyl, (2R)-1-(methylsulphanyl)propan-2-yl, (2S)-1-(methylsulphanyl)propan-2-yl or 2-methyl-1-(methylsulphanyl)propan-2-yl,
   and the agrochemically active salts thereof.

Preference is furthermore given to using, as fungicides, compounds of the formula (I) in which one or more of the symbols have one of the meanings below:
X¹ represents CR³,
X² represents CR⁴,
R³ represents hydrogen, fluorine chlorine, cyano, hydroxyl, OMe, OisoPr, OCF₃, OCF₂CF₂H, SMe, SCF₃, SCF₂H, SO₂Me, SO₂NMe₂, formyl, COMe, COEt, COCH₂CN, CONHMe, CONMe₂, CONHtertBu, CONHCH₂CH═CH₂, CONHPh, CONH-cyclopropyl, CO₂CH₃, NHCOMe, NHCOisoPr, NHCOCF₃, NHCOtertBu, N(C₂H₅)COMe, N(CH₃)COCH₃, NHCHO, NHCO₂Me, NHCO₂Et, NEt₂, cyclopropylamino, [(1-methylcyclopropyl)carbonyl]amino, CH₂COCH₃, CH₂COtertBu, CH₂CO₂Me, methyl, ethyl, 1-methylethyl, CF₃, morpholin-4-ylsulphonyl, 3-methyl-2,5-dioxoimidazolidin-1-yl, 1,3-thiazol-4-yl, 4-(tert-butoxycarbonyl)piperazin-1-yl, acetyl(cyclohexyl)amino, (2,2-dimethylpropanoyl)(methyl)amino or cyclopropyl(trifluoracetyl)amino,
   where the other substituents have one or more of the meanings mentioned above,
   and the agrochemically active salts thereof.

Preference is furthermore given to using, as fungicides, compounds of the formula (I) in which one or more of the symbols have one of the meanings below:
X¹ represents CR³
X² represents nitrogen or CR⁴,
R⁴ represents hydrogen, fluorine, chlorine, cyano, hydroxyl, OMe, OEt, OCF₃, OCF₂CF₂H, OCONHMe, SMe, SO₂NMe₂, formyl, COMe, COEt, COCH₂CN, CONHMe, CONMe₂, CONHtertBu, NHCOMe, NHCOisoPr, NHCOtertBu, NHCOC(CH₃)₂CH₂F, NHCO(C═CH₂)CH₃, N(CH₃)COCH₃, NHCHO, NHCO₂Me, NHCO₂Et, NHCO₂Ph, [(1-methylcyclopropyl)carbonyl]amino, CH₂CN, CH₂COCH₃, CH₂COtertBu, CH₂CO₂Me, methyl, ethyl, 1-methylethyl, CF₃, morpholin-4-ylsulphonyl, (3-methyl-2-oxoimidazolidin-1-yl)methyl, piperidin-1-ylsulphonyl, (benzylsulphamoyl)methyl, (dimethylsulphamoyl)methyl, (furan-2-ylcarbonyl)amino, [(1-phenylethyl)sulphamoyl]methyl, [ethyl(methyl)sulphamoyl]methyl or [methyl(propan-2-yl)sulphamoyl]methyl,
   where the other substituents have one or more of the meanings mentioned above,
   and the agrochemically active salts thereof.

Preference is furthermore given to using, as fungicides, compounds of the formula (I) in which one or more of the symbols have one of the meanings below:
R⁶ represents H, CHO or COCH₃,
R⁷ represents H,
R⁹ represents H, Me or ethyl
   where the other substituents have one or more of the meanings mentioned above,
   and the agrochemically active salts thereof.

Preference is furthermore given to using, as fungicides, compounds of the formula (I) in which one or more of the symbols have one of the meanings below:
R¹ represents hydrogen or fluorine,
R⁵ represents hydrogen or fluorine,
   where the other substituents have one or more of the meanings mentioned above,
   and the agrochemically active salts thereof.

Preference is furthermore given to using, as fungicides, compounds of the formula (I) in which
R⁶ represents hydrogen,
   where the other substituents have one or more of the meanings mentioned above,
   and the agrochemically active salts thereof.

Preference is furthermore given to using, as fungicides, compounds of the formula (I) in which one or more of the symbols have one of the meanings below:
R¹ represents hydrogen or fluorine,
R⁵ represents hydrogen or fluorine,
X¹ represents CR³ and
X² represents CR⁴,
   where the other substituents have one or more of the meanings mentioned above,
   and the agrochemically active salts thereof.

Preference is furthermore given to using, as fungicides, compounds of the formula (I) in which
R⁷ represents hydrogen,
   where the other substituents have one or more of the meanings mentioned above,
   and the agrochemically active salts thereof.

Preference is furthermore given to using, as fungicides, compounds of the formula (I) in which
R⁸ represents chlorine, bromine, CF₃ or cyano,
   where the other substituents have one or more of the meanings mentioned above,
   and the agrochemically active salts thereof.

Preference is furthermore given to using, as fungicides, compounds of the formula (I) in which
R⁹ represents hydrogen, Me or ethyl,
   where the other substituents have one or more of the meanings mentioned above,
   and the agrochemically active salts thereof.

Preference is furthermore given to using, as fungicides, compounds of the formula (I) in which
R¹⁰ represents methyl, ethyl, propanyl, propan-2-yl, butanyl, butan-2-yl, tert-butyl, 4-methylpentan-2-yl, hexan-3-yl, cyclopentyl, cyclohexyl, prop-2-en-1-yl, cyanomethyl, 2-hydroxyethyl, 2-methoxyethyl, 2-ethoxyethyl, 2-(trifluoromethoxy)ethyl, 1-hydroxypropan-2-yl, 1-hydroxybutan-2-yl, 1-methoxypropan-2-yl, 1-methoxybutan-2-yl, 1-methoxy-3-methylbutan-2-yl, 1-methoxypentan-2-yl, 2-(methylsulphanyl)ethyl, (2R)-1-(methylsulphanyl)propan-2-yl, (2S)-1-(methylsulphanyl)propan-2-yl or 2-methyl-1-(methylsulphanyl)propan-2-yl,
   where the other substituents have one or more of the meanings mentioned above,
   and the agrochemically active salts thereof.

Preference is furthermore given to using, as fungicides, compounds of the formula (I) in which
R¹, R⁵, R⁶ and R⁷ represent hydrogen,
   where the other substituents have one or more of the meanings mentioned above,
   and the agrochemically active salts thereof.

The radical definitions mentioned above can be combined with one another as desired. Moreover, individual definitions may not apply.

The invention also provides compounds of the formulae (Ia), (Ib) and (Ic).

The compounds of the formulae (Ia), (Ib) and (Ic) according to the invention and their agrochemically active salts are highly suitable for controlling phytopathogenic harmful fungi. The compounds according to the invention mentioned above have in particular strong fungicidal activity and can be used both in crop protection, in the domestic and hygiene field and in the protection of materials.

Compounds of the formula (Ia),

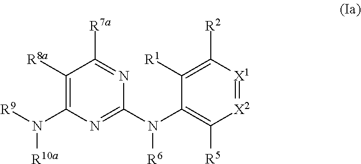

in which
X¹, X², R¹ to R⁶, R⁹, R¹² and R¹³ have the general, preferred, particularly preferred, very particularly preferred and especially preferred meanings given above,
R^{7a} represents hydrogen, C₂-C₃-alkyl, cyano or C₁-C₃-haloalkyl,
R^{8a} represents chlorine, iodine, SMe, SOMe, SOMe, CCl₃, CFH₂ or CF₂H,
R^{10a} represents a group E1 of the formula

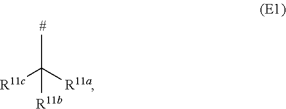

in which the symbols are as defined below and # represents the point of attachment to the nitrogen atom:
R^{11a} represents hydrogen or methyl,
R^{11b} represents hydrogen or C₁-C₃-alkyl,
R^{11c} represents hydrogen, C₁-C₄-alkyl, CN, CH₂CN, CH(Me)CN, C(Me)₂CN, CH₂O—(C₁-C₃-alkyl), CH₂O—(C₁-C₃-haloalkyl), C(Me)₂OH, C(Me)HO—(C₁-C₃-alkyl), C (Me)₂O—(C₁-C₃-alkyl), C(Me)HO—(C₁-C₃-haloalkyl), C(Me)₂O—(C₁-C₃-haloalkyl), CH₂S—(C₁-C₃-alkyl), C(Me)HS—(C₁-C₃-alkyl), C(Me)S—(C₁-C₃-alkyl), Si—(C₁-C₂-alkyl)₃ or C₂-C₄-alkenyl which is optionally substituted by halogen or CF₃,
   or
R^{11c} represents a cyclopentyl or a cyclohexyl ring which is optionally substsituted by CN, halogen, C₁-C₃-alkyl, C₁-C₃-alkoxy, C₁-C₃-haloalkyl, C₁-C₃-haloalkoxy, C₁-C₃-alkoxy-C₁-C₃-alkyl,
   and agrochemically active salts of these compounds.

Compounds of the formula (Ib),

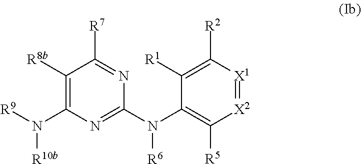

in which
X¹, X², R¹ to R⁷, R⁹, R¹² and R¹³ have the general, preferred, particularly preferred, very particularly preferred and especially preferred meanings given above,
R^{8b} represents CF₃,
R^{10b} represents a group E1 of the formula below

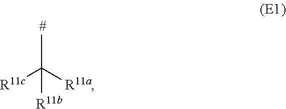

in which the symbols are as defined below and # represents the point of attachment to the nitrogen atom:
R^{11a} represents hydrogen or methyl,
R^{11b} represents hydrogen or C₁-C₃-alkyl,
R^{11c} represents hydrogen, C₁-C₄-alkyl, CN, CH₂CN, CH(Me)CN, C(Me)₂CN, CH₂O—(C₁-C₃-alkyl), CH₂O—(C₁-C₃-haloalkyl), C(Me)₂OH, C(Me)HO—(C₁-C₃-alkyl), C(Me)₂O—(C₁-C₃-alkyl), C(Me)HO—(C₁-C₃-haloalkyl), C(Me)₂O—(C₁-C₃-haloalkyl), C₁₋₂S—(C₁-C₃-alkyl), C(Me)HS—(C₁-C₂-alkyl), C(Me)₂S—(C₁-C₃-alkyl), Si—(C₁-C₂-alkyl)₃ or C₂-C₄-alkenyl which is optionally substituted by halogen or CF₃,

### Or

R^{11c} represents a cyclopentyl or a cyclohexyl ring which is optionally substituted by CN, halogen, C₁-C₃-alkyl, C₁-C₃-alkoxy, C₁-C₃-haloalkyl, C₁-C₃-haloalkoxy, C₁-C₃-alkoxy-C₁-C₃-alkyl;
   except for the cases in which in the subunit of the general formula (In) below

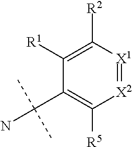

X¹ represents CR³ and X² represents CR⁴ and
R³ and R⁴ together with the phenyl ring form a 1H-2-hydroxyindol-5-yl or 1H-2-hydroxyindol-6-yl radical;
   or R³ represents CON(R¹²)₂ and the two radicals R¹² together with the nitrogen atom to which they are attached form a 4-methyl-1,4-piperazinyl radical;
   or R² and R³ or R³ and R⁴ represent chlorine;
   and agrochemically active salts of these compounds.

Compounds of the formula (Ic),

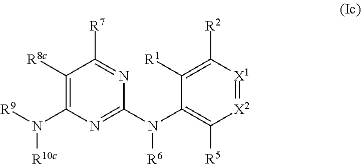

in which
X¹, X², R¹ to R⁷, R⁹, R¹² and R¹³ have the general, preferred, particularly preferred, very particularly preferred and especially preferred meanings given above,
R^{8c} represents bromine and
R^{10c} represents a group E1 of the formula

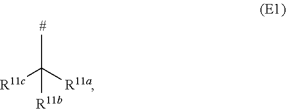

in which the symbols are as defined below and # represents the point of attachment to the nitrogen atom:
R^{11a} represents hydrogen or methyl,
R^{11b} represents hydrogen or C₁-C₃-alkyl,
R^{11c} represents hydrogen, C₁-C₄-alkyl, CN, CH₂CN, CH(Me)CN, C(Me)₂CN, CH₂O—(C₁-C₃-alkyl), CH₂O—(C₁-C₃-haloalkyl), C(Me)₂OH, C(Me)HO—(C₁-C₃-alkyl), C(Me)₂O—(C₁-C₃-alkyl), C(Me)HO—(C₁-C₃-haloalkyl), C(Me)₂O—(C₁-C₃-haloalkyl), CH₂S—(C₁-C₃-alkyl), C(Me)HS—(C₁-C₃-alkyl), C(Me)S—(C₁-C₃-alkyl), Si—(C₁-C₂-alkyl)₃ or C₂-C₄-alkenyl which is optionally substituted by halogen or CF₃,
   or
R^{11c} represents a cyclopentyl or a cyclohexyl ring which is optionally substituted by CN, halogen, C₁-C₃-alkyl, C₁-C₃-alkoxy, C₁-C₃-haloalkoxy, C₁-C₃-alkoxy-C₁-C₃-alkyl;
   except for the cases in which in the subunit of the general formula (Ic) below

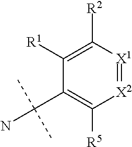

X¹ represents CR³ and X² represents CR⁴ and
R³ and R⁴ together with the phenyl ring form a 1H-2-hydroxyindol-5-yl or a 1H-indazol-5-ylamino radical;
or R³ represents OH, SMe, SOMe, SO₂NMe₂, piperazin-1-yl, 4-tbutyloxycarbonylpiperazin-1-yl or (pyrrolidin-1-yl)methyl;
or R² or R⁴ represents SMe;
   and the agrochemically active salts of these compounds.

The radical definitions mentioned above can be combined with one another as desired. Moreover, individual definitions may not apply.

The compounds of the formulae (I), (Ia), (Ib) and (Ic) can be present in both pure form and as mixtures of various possible isomeric forms, in particular of stereoisomers, such as E and Z, threo and erythro, and also optical isomers, such as R and S isomers or atropisomers, and, if appropriate, also of tautomers. What is claimed are both the E and the Z isomers, and the threo and erythro, and also the optical isomers, any mixtures of these isomers, and also the possible tautomeric forms.

Depending on the nature of the substituents defined above, the compounds of the formulae (I), (Ia), (Ib) and (Ic) have acidic or basic properties and can form salts, if appropriate also inner salts, or adducts with inorganic or organic acids or with bases or with metal ions. If the compounds of the formulae (I), (Ia), (Ib) and (Ic) carry amino, alkylamino or other groups which induce basic properties, these compounds can be reacted with acids to give salts, or they are directly obtained as salts in the synthesis. If the compounds of the formulae (I), (Ia), (Ib) and (Ic) carry hydroxyl, carboxyl or other groups which induce acidic properties, these compounds can be reacted with bases to give salts. Suitable bases are, for example, hydroxides, carbonates, bicarbonates of the alkali metals and alkaline earth metals, in particular those of sodium, potassium, magnesium and calcium, furthermore ammonia, primary, secondary and tertiary amines having (C₁-C₄)-alkyl groups, mono-, di- and trialkanolamines of (C₁-C₄)-alkanols, choline and also chlorocholine.

The salts obtainable in this manner also have fungicidal properties.

Examples of inorganic acids are hydrohalic acids, such as hydrogen fluoride, hydrogen chloride, hydrogen bromide and hydrogen iodide, sulphuric acid, phosphoric acid and nitric acid, and acidic salts, such as NaHSO₄ and KHSO₄. Suitable organic acids are, for example, formic acid, carbonic acid and alkanoic acids, such as acetic acid, trifluoroacetic acid, trichloroacetic acid and propionic acid, and also glycolic acid, thiocyanic acid, lactic acid, succinic acid, citric acid, benzoic acid, cinnamic acid, oxalic acid, alkylsulphonic acids (sulphonic acids having straight-chain or branched alkyl radicals of 1 to 20 carbon atoms), arylsulphonic acids or aryldisulphonic acids (aromatic radicals, such as phenyl and naphthyl, which carry one or two sulphonic acid groups), alkylphosphonic acids (phosphonic acids having straight-chain or branched alkyl radicals of 1 to 20 carbon atoms), arylphosphonic acids or aryldiphosphonic acids (aromatic radicals, such as phenyl and naphthyl, which carry one or two phosphonic acid radicals), where the alkyl and aryl radicals may carry further substituents, for example p-toluenesulphonic acid, salicylic acid, p-aminosalicylic acid, 2-phenoxybenzoic acid, 2-acetoxybenzoic acid, etc.

Suitable metal ions are in particular the ions of the elements of the second main group, in particular calcium and magnesium, of the third and fourth main group, in particular aluminium, tin and lead, and also of the first to eighth transition group, in particular chromium, manganese, iron, cobalt, nickel, copper, zinc and others. Particular preference is given to the metal ions of the elements of the fourth period. Here, the metals can be present in the various valencies that they can assume.

Optionally substituted groups may be mono- or polysubstituted, where in the case of polysubstitution the substituents may be identical or different.

In the definitions of the symbols given in the formulae above, collective terms were used which are generally representative for the following substituents:

halogen: fluorine, chlorine, bromine and iodine;
aryl: an unsubstituted or optionally substituted 5- to 15-membered partially or fully unsaturated mono-, bi- or tricyclic ring system having up to 3 ring members selected from the groups C(═O), (C═S), where at least one of the rings of the ring system is fully unsaturated, such as, for example (but not limited thereto) benzene, naphthalene, tetrahydronaphthalene, anthracene, indane, phenanthrene, azulene;
alkyl: saturated straight-chain or branched hydrocarbon radicals having 1 to 10 carbon atoms, such as, for example (but not limited thereto) methyl, ethyl, propyl, 1-methylethyl, butyl, 1-methyl-propyl, 2-methylpropyl, 1,1-dimethylethyl, pentyl, 1-methylbutyl, 2-methylbutyl, 3-methylbutyl, 2,2-dimethylpropyl, 1-ethylpropyl, hexyl, 1,1-dimethylpropyl, 1,2-dimethylpropyl, 1-methylpentyl, 2-methylpentyl, 3-methylpentyl, 4-methylpentyl, 1,1-dimethylbutyl, 1,2-dimethylbutyl, 1,3-dimethylbutyl, 2,2-dimethylbutyl, 2,3-dimethylbutyl, 3,3-dimethylbutyl, 1-ethylbutyl, 2-ethylbutyl, 1,1,2-trimethylpropyl, 1,2,2-trimethylpropyl, 1-ethyl-1-methylpropyl and 1-ethyl-2-methylpropyl, heptyl, 1-methylhexyl, octyl, 1,1-dimethylhexyl, 2-ethylhexyl, 1-ethylhexyl, nonyl, 1,2,2-trimethylhexyl, decyl;
haloalkyl: straight-chain or branched alkyl groups having 1 to 4 carbon atoms (as mentioned above), where in these groups some or all of the hydrogen atoms may be replaced by halogen atoms as mentioned above, such as, for example (out not limited thereto), C₁-C₂-haloalkyl, such as chloromethyl, bromomethyl, dichloromethyl, trichloromethyl, fluoromethyl, difluoromethyl, trifluoromethyl, chlorofluoromethyl, dichlorofluoromethyl, chlorodifluoromethyl, 1-chloroethyl, 1-bromoethyl, 1-fluoroethyl, 2-fluoroethyl, 2,2-difluoroethyl, 2,2,2-trifluoroethyl, 2-chloro-2-fluoroethyl, 2-chloro-2-difluoroethyl, 2,2-dichloro-2-fluoroethyl, 2,2,2-trichloroethyl, pentafluoroethyl and 1,1,1-trifluoroprop-2-yl;
alkenyl: unsaturated straight-chain or branched hydrocarbon radicals having 2 to 16 carbon atoms and at least one double bond in any position, such as, for example (but not limited thereto), C₂-C₆-alkenyl, such as ethenyl, 1-propenyl, 2-propenyl, 1-methylethenyl, 1-butenyl, 2-butenyl, 3-butenyl, 1-methyl-1-propenyl, 2-methyl-1-propenyl, 1-methyl-2-propenyl, 2-methyl-2-propenyl, 1-pentenyl, 2-pentenyl, 3-pentenyl, 4-pentenyl, 1-methyl-1-butenyl, 2-methyl-1-butenyl, 3-methyl-1-butenyl, 1-methyl-2-butenyl, 2-methyl-2-butenyl, 3-methyl-2-butenyl, 1-methyl-3-butenyl, 2-methyl-3-butenyl, 3-methyl-3-butenyl, 1,1-dimethyl-2-propenyl, 1,2-dimethyl-1-propenyl, 1,2-dimethyl-2-propenyl, 1-ethyl-1-propenyl, 1-ethyl-2-propenyl, 1-hexenyl, 2-hexenyl, 3-hexenyl, 4-hexenyl, 5-hexenyl, 1-methyl-1-pentenyl, 2-methyl-1-pentenyl, 3-methyl-1-pentenyl, 4-methyl-1-pentenyl, 1-methyl-2-pentenyl, 2-methyl-2-pentenyl, 3-methyl-2-pentenyl, 4-methyl-2-pentenyl, 1-methyl-3-pentenyl, 2-methyl-3-pentenyl, 3-methyl-3-pentenyl, 4-methyl-3-pentenyl, 1-methyl-4-pentenyl, 2-methyl-4-pentenyl, 3-methyl-4-pentenyl, 4-methyl-4-pentenyl, 1,1-dimethyl-2-butenyl, 1,1-dimethyl-3-butenyl, 1,2-dimethyl-1-butenyl, 1,2-dimethyl-2-butenyl, 1,2-dimethyl-3-butenyl, 1,3-dimethyl-1-butenyl, 1,3-dimethyl-2-butenyl, 1,3-dimethyl-3-butenyl, 2,2-dimethyl-3-butenyl, 2,3-dimethyl-1-butenyl, 2,3-dimethyl-2-butenyl, 2,3-dimethyl-3-butenyl, 3,3-dimethyl-1-butenyl, 3,3-dimethyl-2-butenyl, 1-ethyl-1-butenyl, 1-ethyl-2-butenyl, 1-ethyl-3-butenyl, 2-ethyl-1-butenyl, 2-ethyl-2-butenyl, 2-ethyl-3-butenyl, 1,1,2-trimethyl-2-propenyl, 1-ethyl-1-methyl-2-propenyl, 1-ethyl-2-methyl-1-propenyl and 1-ethyl-2-methyl-2-propenyl;
alkynyl: straight-chain or branched hydrocarbon groups having 2 to 16 carbon atoms and at least one triple bond in any position, such as, for example (but not limited thereto), C₂-C₆-alkynyl, such as ethynyl, 1-propynyl, 2-propynyl, 1-butynyl, 2-butynyl, 3-butynyl, 1-methyl-2-propynyl, 1-pentynyl, 2-pentynyl, 3-pentynyl, 4-pentynyl, 1-methyl-2-butynyl, 1-methyl-3-butynyl, 2-methyl-3-butynyl, 3-methyl-1-butynyl, 1,1-dimethyl-2-propynyl, 1-ethyl-2-propynyl, 1-hexynyl, 2-hexynyl, 3-hexynyl, 4-hexynyl, 5-hexynyl, 1-methyl-2-pentynyl, 1-methyl-3-pentynyl, 1-methyl-4-pentynyl, 2-methyl-3-pentynyl, 2-methyl-4-pentynyl, 3-methyl-1-pentynyl, 3-methyl-4-pentynyl, 4-methyl-1-pentynyl, 4-methyl-2-pentynyl, 1,1-dimethyl-2-butynyl, 1,1-dimethyl-3-butynyl, 1,2-dimethyl-3-butynyl, 2,2-dimethyl-3-butynyl, 3,3-dimethyl-1-butynyl, 1-ethyl-2-butynyl, 1-ethyl-3-butynyl, 2-ethyl-3-butynyl and 1-ethyl-1-methyl-2-propynyl;
alkoxy: saturated straight-chain or branched alkoxy radicals having 1 to 4 carbon atoms, such as, for example (but not limited thereto), C₁-C₄-alkoxy, such as methoxy, ethoxy, propoxy, 1-methylethoxy, butoxy, 1-methylpropoxy, 2-methylpropoxy, 1,1-dimethylethoxy;
haloalkoxy: straight-chain or branched alkoxy groups having 1 to 4 carbon atoms (as mentioned above), where some or all of the hydrogen atoms in these groups may be replaced by halogen atoms as mentioned above, such as, for example (but not limited thereto), C₁-C₂-haloalkoxy, such as chloromethoxy, bromomethoxy, dichloromethoxy, trichloromethoxy, fluoromethoxy, difluoromethoxy, trifluoromethoxy, chlorofluoromethoxy, dichlorofluoromethoxy, chlorodifluoromethoxy, 1-chloroethoxy, 1-bromoethoxy, 1-fluoroethoxy, 2-fluoroethoxy, 2,2-difluoroethoxy, 2,2,2-trifluoroethoxy, 2-chloro-2-fluoroethoxy, 2-chloro-2,2-difluorethoxy, 2,2-dichloro-2-fluorethoxy, 2,2,2-trichloroethoxy, pentafluoroethoxy and 1,1,1-trifluoroprop-2-oxy;
thioalkyl: saturated straight-chain or branched alkylthio radicals having 1 to 6 carbon atoms, such as, for example (out not limited thereto), C₁-C₆-alkylthio, such as methylthio, ethylthio, propylthio, 1-methylethylthio, butylthio, 1-methylpropylthio, 2-methylpropylthio, 1,1-dimethylethylthio, pentylthio, 1-methylbutylthio, 2-methylbutylthio, 3-methylbutylthio, 2,2-dimethylpropylthio, 1-ethylpropylthio, hexylthio, 1,1-dimethylpropylthio, 1,2-dimethylpropylthio, 1-methylpentylthio, 2-methylpentylthio, 3-methylpentylthio, 4-methylpentylthio, 1,1-dimethylbutylthio, 1,2-dimethylbutylthio, 1,3-dimethylbutylthio, 2,2-dimethylbutylthio, 2,3-dimethylbutylthio, 3,3-dimethylbutylthio, 1-ethylbutylthio, 2-ethylbutylthio, 1,1,2-trimethylpropylthio, 1,2,2-trimethylpropylthio, 1-ethyl-1-methylpropylthio and 1-ethyl-2-methylpropylthio;
thiohaloalkyl: straight-chain or branched alkylthio groups having 1 to 6 carbon atoms (as mentioned above), where some or all of the hydrogen atoms in these groups may be replaced by halogen atoms as mentioned above, such as, for example (but not limited thereto) C₁-C₂-haloalkylthio, such as chloromethylthio, bromomethylthio, dichloromethylthio, trichloromethylthio, fluoromethylthio, difluoromethylthio, trifluoromethylthio, chlorofluoromethylthio, dichlorofluoromethylthio, chlorodifluoromethylthio, 1-chloroethylthio, 1-bromoethylthio, 1-fluoroethylthio, 2-fluoroethylthio, 2,2-difluoroethylthio, 2,2,2-trifluoroethylthio, 2-chloro-2-fluoroethylthio, 2-chloro-2,2-difluoroethylthio, 2,2-dichloro-2-fluoroethylthio, 2,2,2-trichloroethylthio, pentafluoroethylthio and 1,1,1-trifluoroprop-2-ylthio;
cycloalkyl: mono-, bi- or tricyclic saturated hydrocarbon groups having 3 to 12 carbon ring members, such as, for example (but not limited thereto), cyclopropyl, cyclobutyl, cyclopentyl and cyclohexyl, bicyclo[1,0,1]butane, decalinyl, norbornyl;
cycloalkenyl: mono-, bi- or tricyclic non-aromatic hydrocarbon groups having 5 to 15 carbon ring members and at least one double bond, such as, for example (but not limited thereto) cyclopenten-1-yl, cyclohexen-1-yl, cyclohepta-1,3-dien-1-yl, norbornen-1-yl;
(alkoxy)carbonyl: an alkoxy group having 1 to 4 carbon atoms (as mentioned above) which is attached to the skeleton via a carbonyl group (—CO—);
heterocyclyl: a three- to fifteen-membered saturated or partially unsaturated heterocycle which contains one to four heteroatoms from the group consisting of oxygen, nitrogen and sulphur: mono-, bi- or tricyclic heterocycles containing, in addition to carbon ring members, one to three nitrogen atoms and/or one oxygen or sulphur atom or one or two oxygen and/or sulphur atoms; if the ring contains a plurality of oxygen atoms, these are not directly adjacent; such as, for example (but not limited thereto), oxiranyl, aziridinyl, 2-tetrahydrofuranyl, 3-tetrahydrofuranyl, 2-tetrahydrothienyl, 3-tetrahydrothienyl, 2-pyrrolidinyl, 3-pyrrolidinyl, 3-isoxazolidinyl, 4-isoxazolidinyl, 5-isoxazolidinyl, 3-isothiazolidinyl, 4-isothiazolidinyl, 5-isothiazolidinyl, 4-pyrazolidinyl, 5-pyrazolidinyl, 2-oxazolidinyl, 4-oxazolidinyl, 5-oxazolidinyl, 2-thiazolidinyl, 4-thiazolidinyl, 5-thiazolidinyl, 2-imidazolidinyl, 4-imidazolidinyl, 1,2,4-oxadiazolidin-3-yl, 1,2,4-oxadiazolidin-5-yl, 1,2,4-thiadiazolidin-3-yl, 1,2,4-thiadiazolidin-5-yl, 1,2,4-triazolidin-3-yl, 1,3,4-oxadiazolidin-2-yl, 1,3,4-thiadiazolidin-2-yl, 1,3,4-triazolidin-2-yl, 2,3-dihydrofur-2-yl, 2,3-dihydrofur-3-yl, 2,4-dihydrofur-2-yl, 2,4-dihydrofur-3-yl, 2,3-dihydrothien-2-yl, 2,3-dihydrothien-3-yl, 2,4-dihydrothien-2-yl, 2,4-dihydrothien-3-yl, 2-pyrrolin-2-yl, 2-pyrrolin-3-yl, 3-pyrrolin-2-yl, 3-pyrrolin-3-yl, 2-isoxazolin-3-yl, 3-isoxazolin-3-yl, 4-isoxazolin-3-yl, 2-isoxazolin-4-yl, 3-isoxazolin-4-yl, 4-isoxazolin-4-yl, 2-isoxazolin-5-yl, 3-isoxazolin-5-yl, 4-isoxazolin-5-yl, 2-isothiazolin-3-yl, 3-isothiazolin-3-yl, 4-isothiazolin-3-yl, 2-isothiazolin-4-yl, 3-isothiazolin-4-yl, 4-isothiazolin-4-yl, 2-isothiazolin-5-yl, 3-isothiazolin-5-yl, 4-isothiazolin-5-yl, 2,3-dihydropyrazol-1-yl, 2,3-dihydropyrazol-2-yl, 2,3-dihydropyrazol-3-yl, 2,3-dihydropyrazol-4-yl, 2,3-dihydropyrazol-5-yl, 3,4-dihydropyrazol-1-yl, 3,4-dihydropyrazol-3-yl, 3,4-dihydropyrazol-4-yl, 3,4-dihydropyrazol-5-yl, 4,5-dihydropyrazol-1-yl, 4,5-thhydropyrazol-3-yl, 4,5-dihydropyrazol-4-yl, 4,5-dihydropyrazol-5-yl, 2,3-dihydrooxazol-2-yl, 2,3-dihydrooxazol-3-yl, 2,3-dihydrooxazol-4-yl, 2,3-dihydrooxazol-5-yl, 3,4-dihydrooxazol-2-yl, 3,4-dihydrooxazol-3-yl, 3,4-dihydrooxazol-4-yl, 3,4-dihydrooxazol-5-yl, 3,4-dihydrooxazol-2-yl, 3,4-dihydrooxazol-3-yl, 3,4-dihydrooxazol-4-yl, 2-piperidinyl, 3-piperidinyl, 4-piperidinyl, 1,3-dioxan-5-yl, 2-tetra-hydropyranyl, 4-tetrahydropyranyl, 2-tetrahydrothienyl, 3-hexahydro-pyridazinyl, 4-hexa-hydropyridazinyl, 2-hexahydropyrimidinyl, 4-hexahydropyrimidinyl, 5-hexahydropyrimidinyl, 2-piperazinyl, 1,3,5-hexahydrotriazin-2-yl und 1,2,4-hexahydrotriazin-3-yl;
hetaryl: unsubstituted or optionally substituted, 5- to 15-membered partially or fully unsaturated mono-, bi- or tricyclic ring system where at least one of the rings of the ring system is fully unsaturated, comprising one to four heteroatoms from the group consisting of oxygen, nitrogen and sulphur, if the ring contains a plurality of oxygen atoms, these are not directly adjacent;
such as, for example (but not limited thereto),
5-membered heteroaryl which contains one to four nitrogen atoms or one to three nitrogen atoms and one sulphur or oxygen atom: 5-membered heteroaryl groups which, in addition to carbon atoms, may contain one to four nitrogen atom or one to three nitrogen atoms and one sulphur or oxygen atom as ring members, for example 2-furyl, 3-furyl, 2-thienyl, 3-thienyl, 2-pyrrolyl, 3-pyrrolyl, 3-isoxazolyl, 4-isoxazolyl, 5-isoxazolyl, 3-isothiazolyl, 4-isothiazolyl, 5-isothiazolyl, 3-pyrazolyl, 4-pyrazolyl, 5-pyrazolyl, 2-oxazolyl, 4-oxazolyl, 5-oxazolyl, 2-thiazolyl, 4-thiazolyl, 5-thiazolyl, 2-imidazolyl, 4-imidazolyl, 1,2,4-oxadiazol-3-yl, 1,2,4-oxadiazol-5-yl, 1,2,4-thiadiazol-3-yl, 1,2,4-thiadiazol-5-yl, 1,2,4-triazol-3-yl, 1,3,4-oxadiazol-2-yl, 1,3,4-thiadiazol-2-yl and 1,3,4-triazol-2-yl;
benzo-fused 5-membered heteroaryl which contains one to three nitrogen atoms or one nitrogen atom and one oxygen or sulphur atom: 5-membered heteroaryl groups which, in addition to carbon atoms, may contain one to four nitrogen atoms or one to three nitrogen atoms and one sulphur or oxygen atom as ring members, and in which two adjacent carbon ring members or one nitrogen and one adjacent carbon ring member may be bridged by a buta-1,3-diene-1,4-diyl group in which one or two carbon atoms may be replaced by nitrogen atoms; for example benzindolyl, benzimidazolyl, benzothiazolyl, benzopyrazolyl, benzofuryl;
5-membered heteroaryl which contains one to four nitrogen atoms and is attached via nitrogen or benzo-fused 5-membered heteroaryl which contains one to three nitrogen atoms and is attached via nitrogen: 5-membered heteroaryl groups which, in addition to carbon atoms, may contain one to four nitrogen atoms or one to three nitrogen atoms as ring members and in which two adjacent carbon ring members or one nitrogen and one adjacent carbon ring member may be bridged by a buta-1,3-diene-1,4-diyl group in which one or two carbon atoms may be replaced by nitrogen atoms, where these rings are attached to the skeleton via one of the nitrogen ring members, for example 1-pyrrolyl, 1-pyrazolyl, 1,2,4-triazol-1-yl, 1-imidazolyl, 1,2,3-triazol-1-yl, 1,3,4-triazol-1-yl;
6-membered heteroaryl which contains one to three or one to four nitrogen atoms: 6-membered heteroaryl groups which, in addition to carbon atoms, may contain one to three or one to four nitrogen atoms as ring members, for example 2-pyridinyl, 3-pyridinyl, 4-pyridinyl, 3-pyridazinyl, 4-pyridazinyl, 2-pyrimidinyl, 4-pyrimidinyl, 5-pyrimidinyl, 2-pyrazinyl, 1,3,5-triazin-2-yl and 1,2,4-triazin-3-yl;

Not included are combinations which contradict natural laws and which the person skilled in the art would therefore have excluded based on his expert knowledge. Excluded are, for example, ring structures having three or more adjacent oxygen atoms.

The present invention furthermore relates to a process for preparing the diaminopyrimidines of the formulae (Ia), (Ib) and (Ic) according to the invention, comprising at least one of steps (a) to (f) below:
(a) the reaction of 2,4-dihalopyrimidines of the formula (III) with amines of the formula (II) in the presence of a base, if appropriate in the presence of a solvent, if appropriate in the presence of a catalyst, to give compounds of the formula (V), according to the reaction scheme below (Scheme 1):

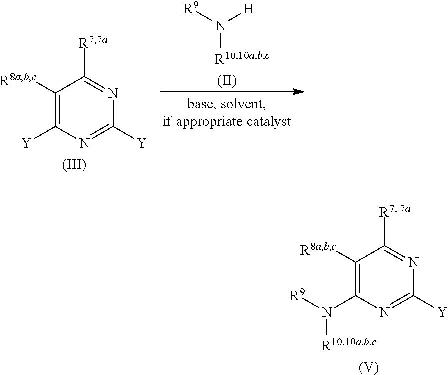

where Y═F, Cl, Br, I
(b) the reaction of compounds of the formula (V) with (het)aromatic amines of the formula (IV), if appropriate in the presence of an acid, if appropriate in the presence of a solvent, according to the reaction scheme below (Scheme 2):

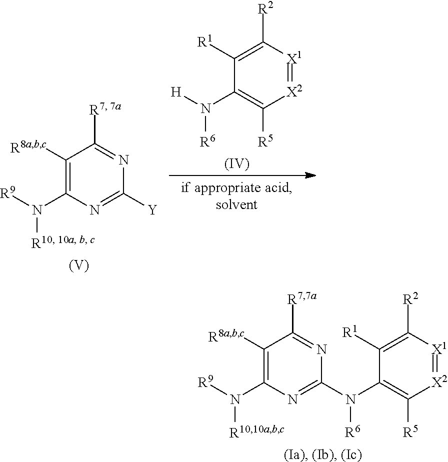

where Y═F, Cl, Br, I
(c) the reaction of compounds of the formula (VIb) with a (het)aromatic amine of the formula (IV), if appropriate in the presence of an acid and if appropriate in the presence of a solvent, according to the reaction scheme below (Scheme 3):

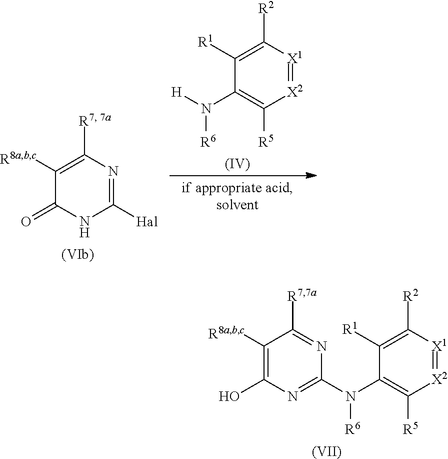

where Hal═F, Cl, Br, I
(d) the reaction of compounds of the formula (VIII) with a halogenating agent, if appropriate in the presence of a solvent, to give compounds of the formula (VIII), according to the reaction scheme below (Scheme 4):

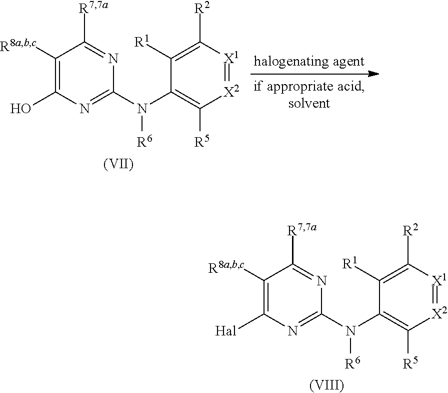

the reaction of compounds of the formula (Mb) with a (het)aromatic amine of the formula (IV), if appropriate in the presence of a catalyst and if appropriate in the presence of a solvent, according to the reaction scheme below (Scheme 5):

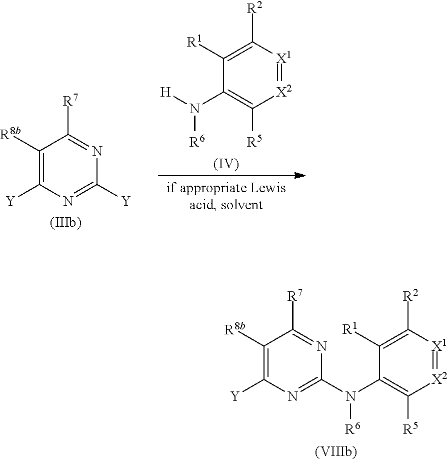

where Y═F, Cl, Br, I and R^{8b}═CF₃
(e) the reaction of compounds of the formula (VIII) with amines of the formula (II) in the presence of a base, if appropriate in the presence of a solvent, if appropriate in the presence of a catalyst, to give compounds of the formulae (Ia, Ib and Ic), according to the reaction scheme below (Scheme 6):

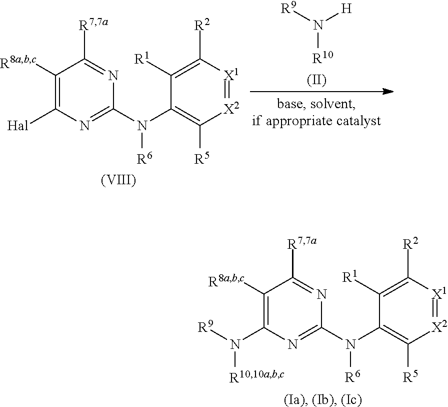

where the definitions of the radicals R¹ to R¹⁰ and X¹ and X² in the above schemes correspond to the definitions and Y and Hal represents F, Cl, Br, I.

One way of preparing the intermediate (V) is shown in Scheme 1.

The alkyl-substituted amino compounds of the formula (II) are either commercially available or can be prepared according to literature procedures. One method of preparing suitable amino compounds (II) is, for example, the reduction of corresponding nitro compounds (described, for example, in J. Chem Soc, 1947, 1474), carboxamides (described, for example, in J. Am. Chem. Soc, 1950, 72, 2657), and the reduction of corresponding azides (described, for example, in Synth. Commun. 1988, 18, 1975), oximes (described, for example, in Org. Lett. 2007, 9, 2665) or nitriles (described, for example, in Org. Lett. 2005, 7, 1737). The synthesis of alkyl-substituted amino compounds of the formula (II) is also possible via reductive amination of ketones or aldehydes (described, for example, in J. Mol Cat, 1999, 146, 129). Further methods for preparing (II) comprise the cleavage of corresponding phthalimides according to Gabriel or the aminolysis of alkyl halides (described, for example, in Tetrahedron Lett. 1999, 40, 4467) and the alkylation of corresponding primary amines (Tetrahedron Assymm. 2000, 11, 1165).

Suitable substituted 2,4-dihalopyrimidines (III) are either commercially available or can be prepared according to literature procedures, for example from commercially available substituted uracils (for example R⁸═CN: J. Org. Chem. 1962, 27, 2264; J. Chem. Soc. 1955, 1834; Chem. Ber. 1909, 42, 734; R⁸═CF₃: J. Fluorine Chem. 1996, 77, 93; see also WO 2000/047539).

Initially, using a suitable base at a temperature of from −30° C. to +80° C. in a suitable solvent, such as, for example, dioxane, THF, dimethylformamide or acetonitrile, an amine (II) is reacted with a 2,4-dihalopyrimidine (III) over a period of 1-24 h. Suitable for use as base are, for example, inorganic salts, such as NaHCO₃, Na₂CO₃ or K₂CO₃, organometallic compounds, such as LDA or NaHMDS, or amine bases, such as ethyldiisopropylamine, DBU, DBN or tri-n-butylamine. Alternatively, the reaction can also be carried out as described, for example, in Org. Lett. 2006, 8, 395 with the aid of a suitable transition metal catalyst, such as, for example, palladium, in combination with a suitable ligand, such as, for example, triphenylphosphine or xanthphos.

Some of the compounds of the formula (V) are novel and thus also form part of the subject-matter of the present invention.

Novel are compounds of the formula (Va)

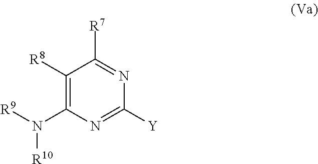

in which
R⁷ represents hydrogen,
R⁸ represents chlorine, iodine, SMe, SOMe, SO₂Me, CF₃, CCl₃, CFH₂ or CF₂H,
R⁹ has the general, preferred, particularly preferred and very particularly preferred meanings as defined above,
Y═F, Cl, Br or I
   and
R¹⁰ represents a group E1 of the formula

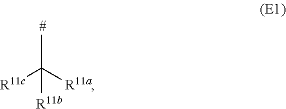

in which the symbols are as defined below and # represents the point of attachment to the nitrogen atom:
R^{11a} represents hydrogen or methyl,
R^{11b} represents hydrogen or C₁-C₃-alkyl,
R^{11c} represents C₂-C₄-alkyl, CN, CH₂CN, CH(Me)CN, C(Me)₂CN, CH₂O—(C₁-C₃-alkyl), CH₂O—(C₁-C₃-haloalkyl), C(Me)₂OH, C(Me)HO—(C₁-C₃-alkyl), C(Me)₂O—(C₁-C₃-alkyl), C(Me)HO—(C₁-C₃-haloalkyl), C(Me)₂O—(C₁-C₃-haloalkyl), CH₂S—(C₁-C₃-alkyl), C(Me)HS—(C₁-C₃-alkyl), C(Me)S—(C₁-C₃-alkyl), Si—(C₁-C₂-alkyl)₃ or C₂-C₄-alkenyl which is optionally substituted by halogen or CF₃,
   or
R^{11c} represents a cyclopentyl ring which may optionally be mono- or polysubstituted by CN, halogen, C₁-C₃-alkyl, C₁-C₃-alkoxy, C₁-C₃-haloalkyl, C₁-C₃-haloalkoxy, C₁-C₃-alkoxy-C₁-C₃-alkyl,
   or
R^{11c} represents a cyclohexyl ring which is substituted by at least one of the radicals from the group consisting of CN, halogen, C₁-C₃-alkyl, C₁-C₃-alkoxy, C₁-C₃-haloalkyl, C₁-C₃-haloalkoxy, C₁-C₃-alkoxy-C₁-C₃-alkyl.

One way of preparing the compounds (Ia), (Ib) and (Ic) is shown in Scheme 2.

The substituted (het)aromatic amines (IV) are either commercially available or can be prepared by methods known from the literature from commercially available precursors. (Het)aromatic amines which carry one or more identical or different substituents in the aromatic moiety can be prepared by a large number of methods described in the relevant literature. In an exemplary manner, some of the methods are mentioned below.

Sulphonamide- or sulphonic ester-substituted (het)arylamines can be prepared, for example, by the reaction, known from the literature, of commercially available aminosulphonic acids with chlorinating agents (for example POCl₃) and subsequent reaction of the sulphochlorides formed with O- or N-nucleophiles.

Two customary methods for preparing N-monoacylated diaminoaromatics are outlined in Scheme 7. Thus, for example, nitroanilines can be converted by standard methods using acyl halides, chloroformic esters or iso(thio)cyanates into the corresponding N-acylnitroaromatics, which can then be reduced by procedures known from the literature to give N-acylaminoaromatics. A further method describes the preparation of the compounds mentioned by transition metal-catalysed crosscoupling of aminohaloaromatics and N-acyl compounds (see, for example, J. Am. Chem. Soc. 2001, 123, 7727).

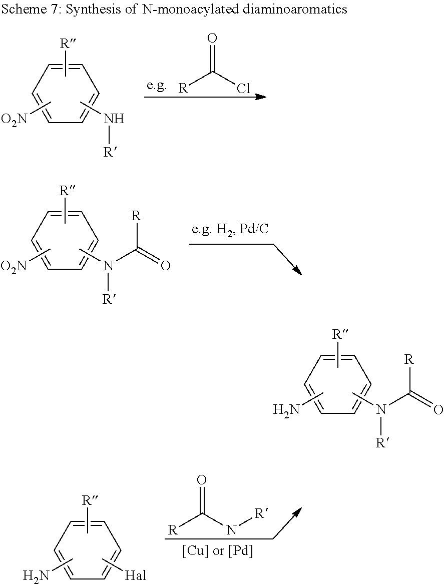

Cyclic radicals R³ or R⁴ attached via nitrogen can be prepared, for example, by condensing nitroaminoaromatics with haloalkylcarbonyl halides or diesters or diester equivalents or lactones; the subsequent reduction of the nitro group affords the desired (het)aromatic amine. A further way to synthesize N-attached radicals R³ or R⁴ is the condensation of nitroarylhydrazines with diesters or diester equivalents, propargyl acid esters or keto esters. The reduction of the nitro group affords the aniline.

Substituted 3- or 4-aminopyridines can be prepared, for example, by reducing appropriately substituted nitropyridines (for example WO2006074147) by Hoffmann or Curtius degradation of corresponding pyridinecarboxylic acid derivatives (for example Bioorg. Med. Chem. Lett. 2006, 16, 3484) or aminolysis of substituted halopyridines (for example EP228846). Other methods consist in the reaction, known from the literature, of corresponding haloaminopyridines with 0-, N- or S-nucleophiles (for example Bioorg. Med. Chem. Lett. 2002, 12, 1517) and also C-nucleophiles (for example Bioorg. Med. Chem. Lett. 2000, 10, 2383). A further method describes the preparation of the compounds mentioned by transition metal-catalysed crosscoupling of aminohalopyridines (WO2006025979).

The intermediate (V) is reacted in the presence of Brönstedt acids, such as, for example, anhydrous hydrochloric acid, camphorsulphonic acid or p-toluenesulphonic acid, in a suitable solvent, such as, for example, dioxane, THF, DMSO, DME, 2-methoxyethanol, n-butanol or acetonitrile, at a temperature of 0° C.-140° C. and over a period of 1-48 h with a (het)aromatic amine (IV). This is analogously described, for example, in Bioorg. Med. Chem. Lett. 2006, 16, 2689; GB2002 A1-2369359, Org. Lett. 2005, 7, 4113.

Alternatively, the reaction of (V) and (IV) to give (Ia), (Ib) or (Ic) can also be carried out under base-catalysis, i.e. using, for example, carbonates, such as potassium carbonate, alkoxides, such as potassium tert-butoxide, or hydrides, such as sodium hydride, where the catalytic use of a transition metal, such as, for example, palladium, together with a suitable ligand, such as, for example, xanthphos, may also be useful.

Finally, it is possible to carry out the reaction of (V) and (IV) to give (I) in the absence of solvents and/or Brönstedt acids (described, for example, in Bioorg. Med. Chem. Lett. 2006, 16, 108; Bioorg. Med. Chem. Lett. 2005, 15, 3881).

One way of preparing compounds of the formula (VII) is shown in Scheme 3.

2-Halo-substituted pyrimidin-4-ones (VIb) can be obtained from 2,4-dihalo-substituted pyrimidines by regioselective hydrolysis. This is described, for example, in Russ. J. Org. Chem. 2006, 42, 580; J. Med. Chem. 1965, 8, 253.

Intermediates of the formula (VIb) are reacted in the presence of Brönstedt acids, such as, for example, anhydrous hydrochloric acid, camphorsulphonic acid or p-toluenesulphonic acid, in a suitable solvent, such as, for example, dioxane, THF, DMSO, DME, 2-methoxyethanol, n-butanol or acetonitrile at a temperature of 0° C.-140° C. over a period of 1-48 h with a (net)aromatic amine (IV).

Alternatively, the reaction of (VIb) and (IV) to give (VII) can also be carried out under base-catalysis, i.e. using, for example, carbonates, such as potassium carbonate, alkoxides, such as potassium tert-butoxide, or hydrides, such as sodium hydride, where the catalytic use of a transition metal, such as, for example, palladium, together with a suitable ligand, such as, for example, xanthphos, may also be useful.

Finally, it is possible to carry out the reaction of (VIb) and (IV) to give (VII) in the absence of solvents and/or Brönstedt acids (described, for example, in Bioorg. Med. Chem. Lett. 2006, 16, 108; Bioorg. Med. Chem. Lett. 2005, 15, 3881).

Some of the compounds of the formula (VII) are novel and thus also form part of the subject-matter of the present invention.

Novel are compounds of the formula (VIIa),

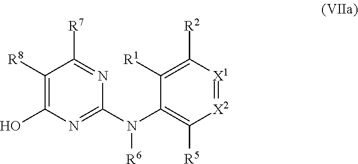

in which the symbols are as defined below:
X¹, X², R¹ to R⁶, R¹² and R¹³ have the general, preferred, particularly preferred, very particularly preferred and especially preferred meanings given above,
R⁷ represents hydrogen
R⁸ represents chlorine, bromine, iodine, CFH₂, CF₂H, CF₃, CCl₃, SMe, SOMe or SO₂Me,
   with the proviso that, if
R¹═R²═R⁵=hydrogen and X²═CH,
R³ must not represent hydrogen, CO₂H, (CH₂)₂OH, SMe, SOMe, or cyano,
   or
   with the proviso that, if
R¹═R⁵=hydrogen and X¹═CH,
   neither R² nor R⁴ must represent OH or CONH₂.

One way of preparing compounds of the formula (VIM is shown in Scheme 4.

Intermediates of the formula (VII) can be converted by reaction with suitable halogenating agents, such as, for example, thionyl chloride, phosphorus pentoxide or phosphoryl chloride or a mixture thereof, if appropriate in the presence of a suitable solvent, such as, for example, toluene or ethanol, and if appropriate in the presence of a suitable base, such as, for example, triethylamine, into 2-anilino-4-chloropyrimidines of the formula (VIII). This is analogously described, for example, in J. Med. Chem. 1989, 32, 1667; J. Heterocycl. Chem. 1989, 26, 313.

A further method for preparing compounds of the formula (VIIIb) from 2,4-dihalopyrimidine (IIIb) and aromatic amines (IV) is shown in Scheme 5.

Using a suitable Lewis acid or a suitable base, at a temperature of from −15° C. to 100° C. and in a suitable inert solvent, such as, for example, 1,4-dioxane, diethyl ether, THF, n-butanol, tert-butanol, dichloroethane or dichloromethane, a (het)aromatic amine (IV) is reacted with a 2,4-dihalopyrimidine (IIIb) over a period of 1-24 h. Suitable for use as base are, for example, inorganic salts, such as NaHCO₃, Na₂CO₃ or K₂CO₃, organometallic compounds, such as LDA or NaHMDS, or amine bases, such as ethyldiisopropylamine, DBU, DBN or tri-n-butylamine. Suitable for use as Lewis acid are, for example (but not limited thereto), halides of the metals zinc (for example ZnCl₂), magnesium, copper, tin or titanium (see, for example, US 2005/0256145 or WO 05/023780 and the literature cited therein).

Some of the compounds of the formula (VIII) are novel and thus also form part of the subject-matter of the present invention.

Novel are compounds of the formula (VIIIa),

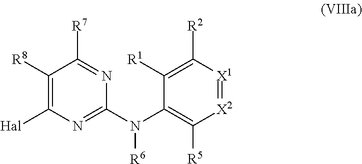

in which the symbols are as defined below:
X¹, X², R² to R⁴, R¹² and R¹³ have the general, preferred, particularly preferred, very particularly preferred and especially preferred meanings given above and
Hal represents fluorine, chlorine, bromine or iodine,
R⁸ represents chlorine, bromine, iodine, Me, CF₃, CFH₂, CF₂H, CCl₃ and cyano,
R¹, R⁵, R⁶ and R⁷ represent hydrogen,
   with the proviso that, if
X²═CH and X¹═CR³,
R³ does not represent CON(Me)-4-(N-methylpiperidinyl), N-piperazinyl, CO-1-(4-methylpiperazinyl), N-morpholinyl, SO₂Me, CONH₂, Me, OMe, COO-benzyl, COOH, CN, NO₂, NMe₂ or Cl,
   or
   with the proviso that, if
X¹═CH and X²═CR⁴,
R² or R⁴ does not represent CN, Cl or 5-oxazolyl,
   or
   with the proviso that, if
X¹═CR³ and X²═CR⁴,
R², R³ and R⁴ do not represent chlorine,
   or
   with the proviso that, if
R⁸═CF₃,
R³ and R⁴ together do not form a saturated or partially unsaturated heterocycle.

A further way of preparing the compound (Ia), (Ib) or (Ic) is shown in Scheme 6.

To prepare compounds of the formula (Ia), (Ib) or (Ic) the intermediate (VIII) is, in the presence of bases, such as, for example, carbonates, such as potassium carbonate, alkoxides, such as potassium tert-butoxide, or hydrides, such as sodium hydride, or amine bases, in a suitable solvent, such as, for example, dioxane, THY, DMSO, DME, 2-methoxyethanol, n-butanol or acetonitrile, at a temperature of 0° C.-140° C. reacted over a period of 1-48 h with amines of the formula (H), where the catalytic use of a transition metal, such as, for example, palladium, together with a suitable ligand, such as, for example, triphenylphosphine or xanthphos, may also be useful.

In general, it is also possible to choose a different route for preparing the compounds (Ia), (Ib), (Ic) and (I) according to the invention, as shown in Scheme 8.

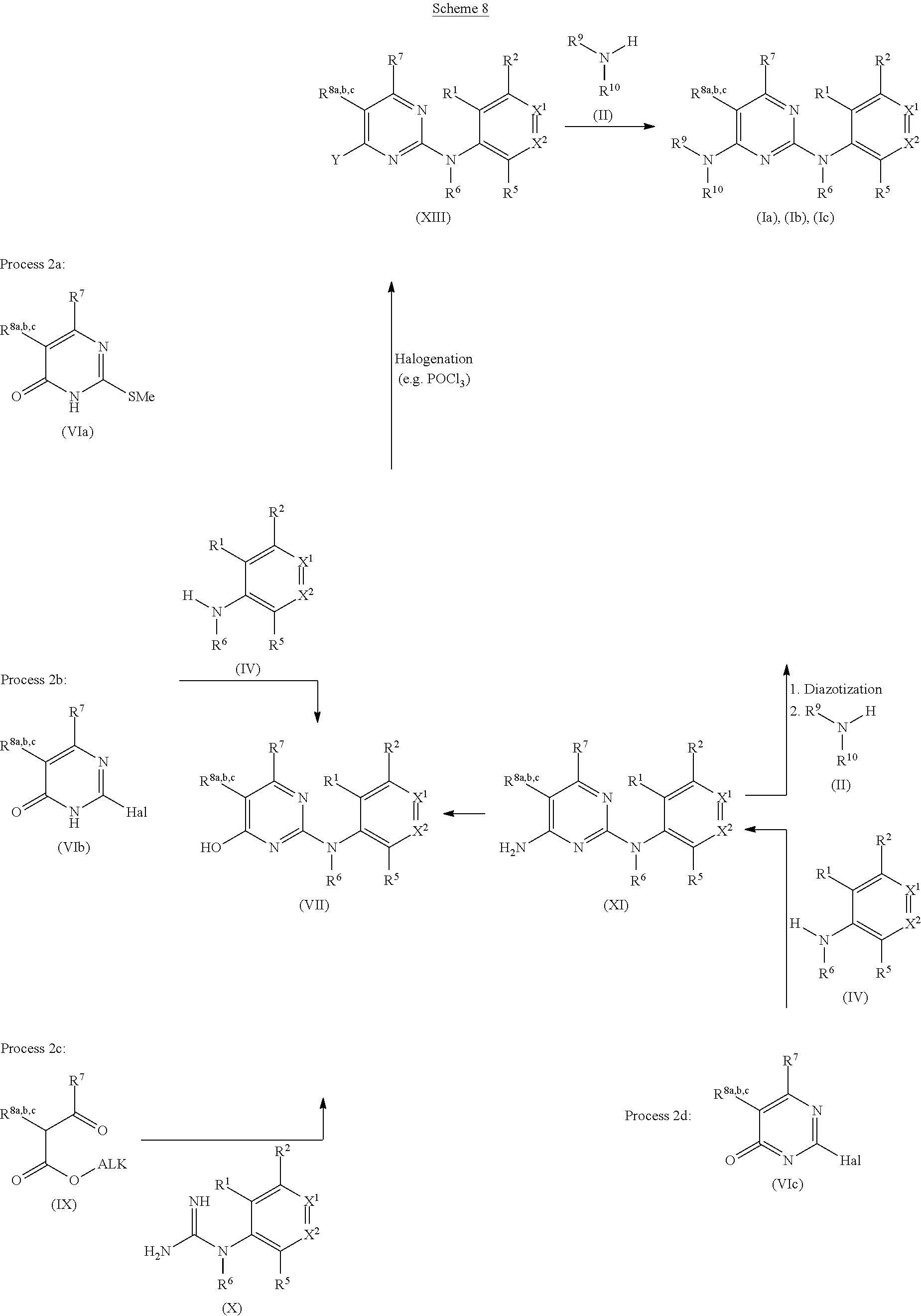

[A further method for preparing diaminopyrimidines of the formulae (Ia), (Ib) and (Ic) and (I) is shown in Scheme 9:

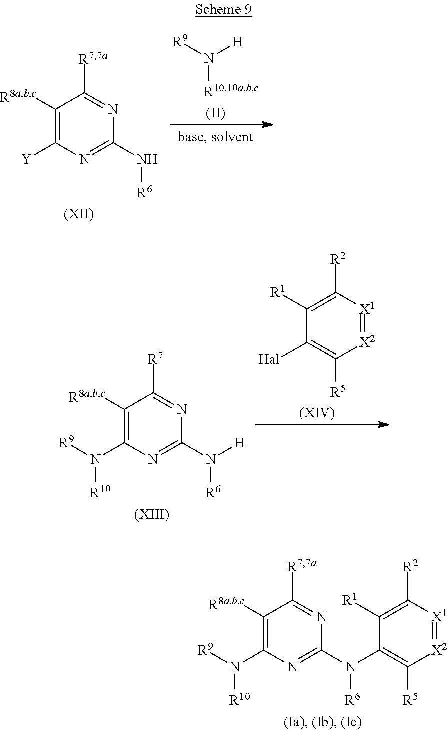

Starting with 4-halo-substituted 2-aminopyrimidines (XII), which can be prepared, for example, analogously to (VIII) from compounds of the type (VIa), (VIb) or (IX) by reaction with R⁶-amines and subsequent chlorination in the 4-position, it is possible to obtain, after addition of an amino compound (II), certain diaminopyrimidines (XIII). In a transition metal-catalysed subsequent step, these can be reacted with an aryl halide (XIV) (as described, for example, in Org. Lett. 2002, 4, 3481), to give the desired target compound (Ia), (Ib), (Ic).

The processes according to the invention for preparing the compounds of the formulae (Ia), (Ib) and (Ic) are preferably carried out using one or more reaction auxiliaries.

Suitable reaction auxiliaries are, if appropriate, the customary inorganic or organic bases or acid acceptors. These preferably include alkali metal and alkaline earth metal acetates, amides, carbonates, bicarbonates, hydrides, hydroxides, and alkoxides, such as, for example, sodium acetate, potassium acetate or calcium acetate, lithium amide, sodium amide, potassium amide or calcium amide, sodium carbonate, potassium carbonate or calcium carbonate, sodium bicarbonate, potassium bicarbonate, or calcium bicarbonate, lithium hydride, sodium hydride, potassium hydride or calcium hydride, lithium hydroxide, sodium hydroxide, potassium hydroxide or calcium hydroxide, sodium methoxide, ethoxide, n- or i-propoxide, n-, s- or t-butoxide or potassium methoxide, ethoxide, n- or i-propoxide, n-, s- or t-butoxide; furthermore also basic organic nitrogen compounds, such as, for example, trimethylamine, triethylamine, tripropylamine, tributylamine, ethyldiisopropylamine, N,N-dimethylcyclohexylamine, dicyclohexylamine, ethyldicyclohexylamine, N,N-dimethylaniline, N,N-dimethylbenzylamine, pyridine, 2-methyl-, 3-methyl-, 4-methyl-, 2,4-dimethyl-, 2,6-dimethyl-, 3,4-dimethyl- and 3,5-dimethylpyridine, 5-ethyl-2-methylpyridine, 4-dimethylaminopyridine, N-methylpiperidine, 1,4-diazabicyclo[2.2.2]-octane (DABCO), 1,5-diazabicyclo[4.3.0]non-5-ene (DBN) or 1,8-diazabicyclo[5.4.0]undec-7-ene (DBU).

The processes according to the invention are preferably carried out using one or more diluents. Suitable diluents are virtually all inert organic solvents. These preferably include aliphatic and aromatic, optionally halogenated hydrocarbons, such as pentane, hexane, heptane, cyclohexane, petroleum ether, benzine, ligroin, benzene, toluene, xylene, methylene chloride, ethylene chloride, chloroform, carbon tetrachloride, chlorobenzene and o-dichlorobenzene, ethers, such as diethyl ether and dibutyl ether, glycol dimethyl ether and diglycol dimethyl ether, tetrahydrofuran and dioxane, ketones, such as acetone, methyl ethyl ketone, methyl isopropyl ketone or methyl isobutyl ketone, esters, such as methyl acetate or ethyl acetate, nitriles, such as, for example, acetonitrile or propionitrile, amides, such as, for example, dimethylformamide, dimethylacetamide and N-methylpyrrolidone, and also dimethyl sulphoxide, tetramethylene sulphone and hexamethyl-phosphoric triamide and DMPU.

In the processes according to the invention, the reaction temperatures can be varied within a relatively wide range. In general, the processes are carried out at temperatures between 0° C. and 250° C., preferably at temperatures between 10° C. and 185° C.

The processes according to the invention are generally carried out under atmospheric pressure. However, it is also possible to operate under elevated or reduced pressure.

For carrying out the processes according to the invention, the starting materials required in each case are generally employed in approximately equimolar amounts. However, it is also possible to use in each case one of the components employed in a relatively large excess. Work-up in the processes according to the invention is in each case carried out by customary methods (cf. the Preparation Examples).

In general, compounds of the formula (I) can be prepared, for example, by sequential nucleophilic addition of an aliphatic amine (II) and a (hetero)aromatic amine (IV) to a suitable substituted pyrimidine (III), as outlined below in Scheme 10:

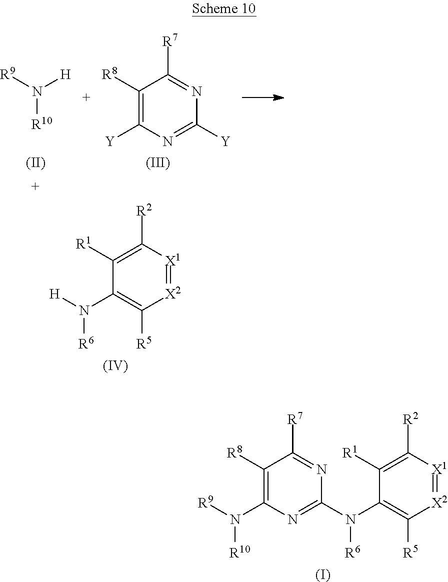

Here, Y, in each case independently of one another, represent suitable leaving groups, for example a halogen atom (Hal═F, Cl, Br, I), SMe, SO₂Me, SOMe or else triflate (CF₃SO₂O: for pyrimidines known from WO 05/095386).

The synthesis of diaminopyrimidines of the formula (I) according to Scheme 8 or else by other routes has been described in the literature in many different instances (see, for example, also WO 06/087230, WO 05/111022, WO 05/111023, WO 05/037800, WO 04/065378, WO 04/056786, WO 04/056807, WO 04/048343, WO 03/032997, WO 03/030909, WO 02/096888).

The invention furthermore provides the non-medicinal use of the diaminopyrimidines according to the invention or of mixtures of these for controlling unwanted microorganisms.

The invention furthermore provides a composition for controlling unwanted microorganisms, comprising at least one diaminopyrimidine according to the present invention.

Moreover, the invention relates to a method for controlling unwanted microorganisms, characterized in that the diaminopyrimidines according to the invention are applied to the microorganims and/or their habitat.

The compounds according to the invention have strong microbicidal action and can be used for controlling unwanted microorganisms, such as fungi and bacteria, in crop protection and in the protection of materials.

The diaminopyrimidines of the formula (I) according to the invention have very good fungicidal properties and can be used in crop protection, for example for controlling Plasmodiophoromycetes, Oornycetes, Chytridiomycetes, Zygomycetes, Ascomycetes, Basidiomycetes and Deuteromycetes.

In crop protection, bactericides can be used for controlling Pseudomonadaceae, Rhizobiaceae, Enterobacteriaceae, Corynebacteriaceae and Streptomycetaceae.

The fungicidal compositions according to the invention can be used for the curative or protective control of phytopathogenic fungi. Accordingly, the invention also relates to curative and protective methods for controlling phytopathogenic fungi using the active compounds or compositions according to the invention, which are applied to the seed, the plant or plant parts, the fruit or the soil on which the plants grow.

The compositions according to the invention for controlling phytopathogenic fungi in crop protection comprise an effective, but non-phytotoxic amount of the active compounds according to the invention. “Effective, but non-phytotoxic amount” means an amount of the composition according to the invention which is sufficient to control the fungal disease of the plant in a satisfactory manner or to eradicate the fungal disease completely, and which, at the same time, does not cause any significant symptoms of phytotoxicity. In general, this application rate may vary within a relatively wide range. It depends on a plurality of factors, for example on the fungus to be controlled, the plant, the climatic conditions and the ingredients of the compositions according to the invention.

According to the invention, it is possible to treat all plants and parts of plants. Plants are to be understood here as meaning all plants and plant populations, such as wanted and unwanted wild plants or crop plants (including naturally occurring crop plants). Crop plants can be plants which can be obtained by conventional breeding and optimization methods or by biotechnological and genetic engineering methods or combinations of these methods, including the transgenic plants and including plant cultivars which can or cannot be protected by varietal property rights. Parts of plants are to be understood as meaning all above-ground and below-ground parts and organs of the plants, such as shoot, leaf, flower and root, examples which may be mentioned being leaves, needles, stems, trunks, flowers, fruit bodies, fruits and seeds and also roots, tubers and rhizomes. Plant parts also include harvested material and vegetative and generative propagation material, for example seedlings, tubers, rhizomes, cuttings and seeds.

The following plants may be mentioned as plants which can be treated according to the invention: cotton, flax, grapevines, fruit, vegetables, such as Rosaceae sp. (for example pomaceous fruit, such as apples and pears, but also stone fruit, such as apricots, cherries, almonds and peaches and soft fruit such as strawberries), Ribesioidae sp., fuglandaceae sp., Betulaceae sp., Anacardiaceae sp., Fagaceae sp., Moraceae sp., Oleaceae sp., Actimidaceae sp., Lauraceae sp., Musaceae sp. (for example banana trees and plantations), Rubiaceae sp. (for example coffee), Theaceae sp., Sterculiceae sp., Rutaceae sp. (for example lemons, oranges and grapefruit), Solanaceae sp. (for example tomatoes), Liliaceae sp., Asteraceae sp. (for example lettuce), Umbelliferae sp., Cruciferae sp., Chenopodiaceae sp., Cucurbitaceae sp. (for example cucumbers), Alliaceae sp. (for example leek, onions), Papilionaceae sp. (for example peas); major crop plants, such Gramineae sp. (for example maize, lawn, cereals such as wheat, rye, rice, barley, oats, millet and triticale), Asteraceae sp. (for example sunflowers), Brassicaceae sp. (for example white cabbage, red cabbage, broccoli, cauliflowers, brussel sprouts, pak choi, kohlrabi, garden radish, and also oilseed rape, mustard, horseradish and cress), Fabacae sp. (for example beans, peas), Papilionaceae sp. (for example soya beans), Solanaceae sp. (for example potatoes), Chenopodiaceae sp. (for example sugarbeet, fodderbeet, swiss chard, beetroot); crop plants and ornamental plants in garden and forest; and also in each case genetically modified varieties of these plants. Preferably, cereal plants are treated according to the invention.

Some pathogens of fungal diseases which can be treated according to the invention may be mentioned by way of example, but not by way of limitation:

Diseases caused by powdery mildew pathogens, such as, for example, Blumeria species, such as, for example, Blumeria graminis; Podosphaera species, such as, for example, Podosphaera leuco-tricha; Sphaerotheca species, such as, for example, Sphaerotheca fuliginea; Uncinula species, such as, for example, Uncinula necator;

Diseases caused by rust disease pathogens, such as, for example, Gymnosporangium species, such as, for example, Gymnosporangium sabinae; Hemileia species, such as, for example, Hemileia vastatrix; Phakopsora species, such as, for example, Phakopsora pachyrhizi and Phakopsora meibomiae; Puccinia species, such as, for example, Puccinia recondita or Puccinia triticina; Uromyces species, such as, for example, Uromyces appendiculatus;

Diseases caused by pathogens from the group of the Oomycetes, such as, for example, Bremia species, such as, for example, Bremia lactucae; Peronospora species, such as, for example, Peronospora pisi or P. brassicae; Phytophthora species, such as, for example Phytophthora infestans; Plasmopara species, such as, for example, Plasmopara viticola; Pseudoperonospora species, such as, for example, Pseudoperonospora humuli or Pseudoperonospora cubensis; Pythium species, such as, for example, Pythium ultimum;

Leaf blotch diseases and leaf wilt diseases caused, for example, by Alternaria species, such as, for example, Alternaria solani; Cercospora species, such as, for example, Cercospora beticola; Cladiosporium species, such as, for example, Cladiosporium cucumerinum; Cochliobolus species, such as, for example, Cochliobolus sativus (conidia form: Drechslera, Syn: Helminthosporium); Colletotrichum species, such as, for example, Colletotrichum lindemuthanium; Cycloconium species, such as, for example, Cycloconium oleaginum; Diaporthe species, such as, for example, Diaporthe citri; Elsinoe species, such as, for example, Elsinoe fawcettii; Gloeosporium species, such as, for example, Gloeosporium laeticolor; Glomerella species, such as, for example, Glomerella cingulata; Guignardia species, such as, for example, Guignardia bidwelli; Leptosphaeria species, such as, for example, Leptosphaeria maculans; Magnaporthe species, such as, for example, Magnaporthe grisea; Microdochium species, such as, for example, Microdochium nivale; Mycosphaerella species, such as, for example, Mycosphaerella graminicola and M. fijiensis; Phaeosphaeria species, such as, for example, Phaeosphaeria nodorum; Pyrenophora species, such as, for example, Pyrenophora teres; Ramularia species, such as, for example, Ramularia collocygni; Rhynchosporium species, such as, for example, Rhynchosporium secalis; Septoria species, such as, for example, Septoria apii; Typhula species, such as, for example, Typhula incarnata; Venturia species, such as, for example, Venturia inaequalis;

Root and stem diseases caused, for example, by Corticium species, such as, for example, Corticium graminearum; Fusarium species, such as, for example, Fusarium oxysporum; Gaeumannomyces species, such as, for example, Gaeumannomyces graminis; Rhizoctonia species, such as, for example Rhizoctonia solani; Tapesia species, such as, for example, Tapesia acuformis; Thielaviopsis species, such as, for example, Thielaviopsis basicola;

Ear and panicle diseases (including maize cobs) caused, for example, by Alternaria species, such as, for example, Alternaria spp.; Aspergillus species, such as, for example, Aspergillus flavus; Cladosporium species, such as, for example, Cladosporium cladosporioides; Claviceps species, such as, for example, Claviceps purpurea; Fusarium species, such as, for example, Fusarium culmorum; Gibberella species, such as, for example, Gibberella zeae; Monographella species, such as, for example, Monographella nivalis; Septoria species, such as for example, Septoria nodorum;

Diseases caused by smut fungi, such as, for example, Sphacelotheca species, such as, for example, Sphacelotheca reiliana; Tilletia species, such as, for example, Tilletia caries; T. controversa; Urocystis species, such as, for example, Urocystis occulta; Ustilago species, such as, for example, Ustilago nuda; U. nuda tritici;

Fruit rot caused, for example, by Aspergillus species, such as, for example, Aspergillus flavus; Botrytis species, such as, for example, Botrytis cinerea; Penicillium species, such as, for example, Penicillium expansum and P. purpurogenum; Sclerotinia species, such as, for example, Sclerotinia sclerotiorum;

Verticilium species, such as, for example, Verticilium alboatrum;

Seed- and soil-borne rot and wilt diseases, and also diseases of seedlings, caused, for example, by Fusarium species, such as, for example, Fusarium culmorum; Phytophthora species, such as, for example, Phytophthora cactorum; Pythium species, such as, for example, Pythium ultimum; Rhizoctonia species, such as, for example, Rhizoctonia solani; Sclerotium species, such as, for example, Sclerotium rolfsii;

Cancerous diseases, galls and witches' broom caused, for example, by Nectria species, such as, for example, Nectria galligena;

Wilt diseases caused, for example, by Monilinia species, such as, for example, Monilinia laxa;

Deformations of leaves, flowers and fruits caused, for example, by Taphrina species, such as, for example, Taphrina deformans;

Degenerative diseases of woody plants caused, for example, by Esca species, such as, for example, Phaemoniella clamydospora and Phaeoacremonium aleophilum and Fomitiporia mediterranea;

Diseases of flowers and seeds caused, for example, by Botrytis species, such as, for example, Botrytis cinerea;

Diseases of plant tubers caused, for example, by Rhizoctonia species, such as, for example, Rhizoctonia solani; Helminthosporium species, such as, for example, Helminthosporium solani;

Diseases caused by bacteriopathogens, such as, for example, Xanthomonas species, such as, for example, Xanthomonas campestris pv. oryzae; Pseudomonas species, such as, for example, Pseudomonas syringae pv. lachrymans; Erwinia species, such as, for example, Erwinia amylovora.

Preference is given to controlling the following diseases of soya beans:

fungal diseases on leaves, stems, pods and seeds caused, for example, by alternaria leaf spot (Alternaria spec. atrans tenuissima), anthracnose (Colletotrichum gloeosporoides dematium var. truncatum), brown spot (Septoria glycines), cercospora leaf spot and blight (Cercospora kikuchii), choanephora leaf blight (Choanephora infundibulifera trispora (Syn.)), dactuliophora leaf spot (Dactuliophora glycines), downy mildew (Peronospora manshurica), drechslera blight (Drechslera glycini), frogeye leaf spot (Cercospora sojina), leptosphaerulina leaf spot (Leptosphaerulina trifolii), phyllostica leaf spot (Phyllosticta sojaecola), pod and stem blight (Phomopsis sojae), powdery mildew (Microsphaera diffusa), pyrenochaeta leaf spot (Pyrenochaeta glycines), rhizoctonia aerial, foliage, and web blight (Rhizoctonia solani), rust (Phakopsora pachyrhizi Phakopsora meibomiae), scab (Sphaceloma glycines), stemphylium leaf blight (Stemphylium botryosum), target spot (Corynespora cassiicola).

Fungal diseases on roots and the stem base caused, for example, by black root rot (Calonectria crotalariae), charcoal rot (Macrophomina phaseolina), fusarium blight or wilt, root rot, and pod and collar rot (Fusarium oxysporum, Fusarium orthoceras, Fusarium semitectum, Fusarium equiseti), mycoleptodiscus root rot (Mycoleptodiscus terrestris), neocosmospora (Neocosmopspora vasinfecta), pod and stem blight (Diaporthe phaseolorum), stem canker (Diaporthe phaseolorum var. caulivora), phytophthora rot (Phytophthora megasperma), brown stem rot (Phialophorao gregata), pythium rot (Pythium aphanidermatum, Pythium irregulare, Pythium debaryanum, Pythium myriotylum, Pythium ultimum), rhizoctonia root rot, stem decay, and damping-off (Rhizoctonia solani), sclerotinia stem decay (Sclerotinia sclerotiorum), sclerotinia southern blight (Sclerotinia rolfsii), thielaviopsis root rot (Thielaviopsis basicola).

In the present case, undesired microorganisms are understood as meaning phytopathogenic fungi and bacteria. Thus, the substances according to the invention can be employed for protecting plants against attack by the abovementioned pathogens within a certain period of time after the treatment. The period of time within which their protection is effected is generally extended from 1 to 10 days, preferably 1 to 7 days, after the plants have been treated with the active compounds.

The fact that the active compounds, at the concentrations required for the controlling of plant diseases, are well tolerated by plants permits the treatment of aerial plant parts, of vegetative propagation material and seed, and of the soil.

In this context, the active compounds according to the invention can be employed particularly successfully for controlling cereal diseases such as, for example, against Erysiphe species, against Puccinia and against Fusaria species, rice diseases such as, for example against Pyricularia and Rhizoctonia and diseases in viticulture, fruit production and vegetable production such as, for example, against Botrytis, Venturia, Sphaerotheca and Podosphaera species.

The active compounds according to the invention are also suitable for increasing the yield. Moreover, they display a low degree of toxicity and are well tolerated by plants.

If appropriate, the compounds according to the invention can, at certain concentrations or application rates, also be used as herbicides, safeners, growth regulators or agents to improve plant properties, or as microbicides, for example as fungicides, antimycotics, bactericides, viricides (including agents against viroids) or as agents against MLO (Mycoplasma-like organisms) and RLO (Rickettsia-like organisms). If appropriate, they can also be employed as insecticides. If appropriate, they can also be employed as intermediates or precursors for the synthesis of other active compounds.

If appropriate, the active compounds according to the invention can also be used in certain concentrations and application rates as herbicides and for influencing plant growth. If appropriate, they can also be employed as intermediates and precursors for the synthesis of further active compounds.

The active compounds according to the invention, in combination with good plant tolerance and favourable toxicity to warm-blooded animals and being tolerated well by the environment, are suitable for protecting plants and plant organisms, for increasing harvest yields and for improving the quality of harvested material in agriculture, in horticulture, in animal husbandry, in forests, in gardens and leisure facilities, in the protection of stored products and of materials, and in the hygiene sector. They are preferably employed as plant protection agents. They are active against normally sensitive and resistant species and against all or some stages of development.

The treatment according to the invention of the plants and plant parts with the active compounds or compositions is carried out directly or by action on their surroundings, habitat or storage space using customary treatment methods, for example by dipping, spraying, atomizing, irrigating, evaporating, dusting, fogging, broadcasting, foaming, painting, spreading-on, watering (drenching), drip irrigating and, in the case of propagation material, in particular in the case of seeds, furthermore as a powder for dry seed treatment, a solution for wet seed treatment, a water-soluble powder for slurry treatment, by encrusting, by coating with one or more coats, etc. It is furthermore possible to apply the active compounds by the ultra-low-volume method or to inject the active compound preparation or the active compound itself into the soil.

The active compounds according to the invention can also be used as defoliants, desiccants, haulm killers and in particular as weed killers. Weeds in the broadest sense are to be understood as meaning all plants which grow in locations when they are unwanted. Whether the compounds according to the invention act as total or selective herbicides depends essentially on the amount applied.

In addition, by the treatment according to the invention it is possible to reuse the mycotoxin content in the harvested material and the food- and feedstuffs prepared therefrom. Particular, but not exclusive, mention may be made here of the following mycotoxins: deoxynivalenol (DON), nivalenol, 15-Ac-DON, 3-Ac-DON, T2- and HT2-toxin, fumonisins, zearalenone, moniliformin, fusarin, diacetoxyscirpenol (DAS), beauvericin, enniatin, fusaroproliferin, fusarenol, ochratoxins, patulin, ergot alkaloids and aflatoxins produced, for example, by the following fungi: Fusarium spec., such as Fusarium acuminatum, F. avenaceum, F. crook wellense, F. culmorum, F. graminearum (Gibberella zeae), F. equiseti, F. fujikoroi, F. musarum, F. oxysporum, F. proliferatum, F. poae, F. pseudograminearum, F. sambucinum, F. scirpi, F. semitectum, F. solani, F. sporotrichoides, F. langsethiae, F. subglutinans, F. tricinctum, F. verticillioides, inter alia, and also by Aspergillus spec., Penicillium spec., Claviceps purpurea, Stachybotrys spec., inter alia.

In the protection of materials, the compositions or active compounds according to the invention can furthermore be employed for protecting industrial materials against attack and destruction by unwanted microorganisms, such as, for example, fungi.

In the present context, industrial materials are understood as meaning nonlife materials which have been made for use in technology. For example, industrial materials which are to be protected by active compounds according to the invention from microbial modification or destruction can be glues, sizes, paper and board, textiles, leather, timber, paints and plastic articles, cooling lubricants and other materials which are capable of being attacked or destroyed by microorganisms. Parts of production plants, for example cooling-water circuits, which can be adversely affected by the multiplication of microorganisms may also be mentioned within the materials to be protected. Industrial materials which may be mentioned with preference for the purposes of the present invention are glues, sizes, paper and board, leather, timber, paints, cooling lubricants and heat-transfer fluids, especially preferably wood. The compositions or active compounds according to the invention can prevent disadvantageous effects such as rotting, decay, discoloration, decoloration or the formation of mould.

The method according to the invention for controlling unwanted fungi can also be employed for protecting storage goods. Here, storage goods are to be understood as meaning natural substances of vegetable or animal origin or process products thereof of natural origin, for which long-term protection is desired. Storage goods of vegetable origin, such as, for example, plants or plant parts, such as stems, leaves, tubers, seeds, fruits, grains, can be protected freshly harvested or after processing by (pre)drying, moistening, comminuting, grinding, pressing or roasting. Storage goods also include timber, both unprocessed, such as construction timber, electricity poles and barriers, or in the form of finished products, such as furniture. Storage goods of animal origin are, for example, hides, leather, furs and hairs. The active compound combinations according to the invention can prevent disadvantageous effects, such as rotting, decay, discoloration, decoloration or the formation of mould.

Microorganisms capable of degrading or changing the industrial materials which may be mentioned are, for example, bacteria, fungi, yeasts, algae and slime organisms. The active compounds according to the invention preferably act against fungi, in particular moulds, wood-discoloring and wood-destroying fungi (Basidiomycetes) and against slime organisms and algae. Microorganisms of the following genera may be mentioned as examples: Alternaria, such as Alternaria tenuis; Aspergillus, such as Aspergillus niger; Chaetomium, such as Chaetomium globosum; Coniophora, such as Coniophora puetana; Lentinus, such as Lentinus tigrinus; Penicillium, such as Penicillium glaucum; Polyporus, such as Polyporus versicolor; Aureobasidium, such as Aureobasidium pullulans; Sclerophoma, such as Sclerophoma pityophila; Trichoderma, such as Trichoderma viride; Escherichia, such as Escherichia coli; Pseudomonas, such as Pseudomonas aeruginosa; Staphylococcus, such as Staphylococcus aureus.

The present invention furthermore relates to a composition for controlling unwanted microorganisms comprising at least one of the diaminopyrimidines according to the invention. These are preferably fungicidal compositions comprising auxiliaries, solvents, carriers, surfactants or extenders suitable for use in agriculture.

According to the invention, a carrier is a natural or synthetic, organic or inorganic substance with which the active compounds are mixed or bonded for better applicability, in particular for application to plants or parts of plants or seed. The carrier, which may be solid or liquid, is generally inert and should be suitable for use in agriculture.

Suitable solid carriers are: for example ammonium salts and ground natural minerals, such as kaolins, clays, talc, chalk, quartz, attapulgite, montmorillonite or diatomaceous earth, and ground synthetic minerals, such as finely divided silica, alumina and silicates; suitable solid carriers for granules are: for example crushed and fractionated natural rocks, such as calcite, marble, pumice, sepiolite and dolomite, and also synthetic granules of inorganic and organic meals, and granules of organic material, such as paper, sawdust, coconut shells, maize cobs and tobacco stalks; suitable emulsifiers and/or foam-formers are: for example nonionic and anionic emulsifiers, such as polyoxyethylene fatty acid esters, polyoxyethylene fatty alcohol ethers, for example alkylaryl polyglycol ethers, alkylsulphonates, alkyl sulphates, arylsulphonates and also protein hydrolysates; suitable dispersants are nonionic and/or ionic substances, for example from the classes of the alcohol/POE and/or POP ethers, acid and/or POP/POE esters, alkylaryl and/or POP/POE ethers, fat and/or POP/POE adducts, POE and/or POP polyol derivatives, POE and/or POP/sorbitan or sugar adducts, alkyl or aryl sulphates, sulphonates and phosphates, or the corresponding PO ether adducts. Furthermore suitable oligo- or polymers, for example those derived from vinylic monomers, from acrylic acid, from EO and/or PO alone or in combination with, for example, (poly)alcohols or (poly)amines. It is also possible to employ lignin and its sulphonic acid derivatives, unmodified and modified celluloses, aromatic and/or aliphatic sulphonic acids and their adducts with formaldehyde.

The active compounds can be converted to the customary formulations, such as solutions, emulsions, wettable powders, water- and oil-based suspensions, powders, dusts, pastes, soluble powders, soluble granules, granules for broadcasting, suspension-emulsion concentrates, natural materials impregnated with active compound, synthetic materials impregnated with active compound, fertilizers and also microencapsulations in polymeric substances.

The active compounds can be used as such, in the form of their formulations or the use forms prepared therefrom, such as ready-to-use solutions, emulsions, water- or oil-based suspensions, powders, wettable powders, pastes, soluble powders, dusts, soluble granules, granules for broadcasting, suspension-emulsion concentrates, natural materials impregnated with active compound, synthetic materials impregnated with active compound, fertilizers and also microencapsulations in polymeric substances. Application is carried out in a customary manner, for example by pouring, spraying, atomizing, broadcasting, dusting, foaming, painting-on, etc. It is furthermore possible to apply the active compounds by the ultra-low-volume method or to inject the preparation of active compound or the active compound itself into the soil. It is also possible to treat the seed of the plants.

The formulations mentioned can be prepared in a manner known per se, for example by mixing the active compounds with at least one customary extender, solvent or diluent, emulsifier, dispersant and/or binder or fixative, wetting agent, water repellant, if appropriate siccatives and UV stabilizers and if appropriate colorants and pigments, antifoams, preservatives, secondary thickeners, glues, gibberellins and other processing auxiliaries.

The compositions according to the invention include not only formulations which are already ready to use and can be applied to the plant or the seed using a suitable apparatus, but also commercial concentrates which have to be diluted with water prior to use.

The active compounds according to the invention can be present as such or in their (commercial) formulations and also in the use forms prepared from these formulations as a mixture with other (known) active compounds, such as insecticides, attractants, sterilants, bactericides, acaricides, nematicides, fungicides, growth regulators, herbicides, fertilizers, safeners and/or semiochemicals.

Suitable for use as auxiliaries are substances which are suitable for imparting to the composition itself and/or to preparations derived therefrom (for example spray liquors, seed dressings) particular properties such as certain technical properties and/or also particular biological properties. Typical suitable auxiliaries are: extenders, solvents and carriers.

Suitable extenders are, for example, water, polar and nonpolar organic chemical liquids, for example from the classes of the aromatic and non-aromatic hydrocarbons (such as paraffins, alkylbenzenes, alkylnaphthalenes, chlorobenzenes), the alcohols and polyols (which, if appropriate, may also be substituted, etherified and/or esterified), the ketones (such as acetone, cyclohexanone), esters (including fats and oils) and (poly)ethers, the unsubstituted and substituted amines, amides, lactams (such as N-alkylpynolidones) and lactones, the sulphones and sulphoxides (such as dimethyl sulphoxide).

Liquefied gaseous extenders or carriers are liquids which are gaseous at ambient temperature and under atmospheric pressure, for example aerosol propellants, such as halogenated hydrocarbons, and also butane, propane, nitrogen and carbon dioxide.

Tackifiers, such as carboxymethylcellulose and natural and synthetic polymers in the form of powders, granules and latices, such as gum arabic, polyvinyl alcohol, polyvinyl acetate, or else natural phospholipids, such as cephalins and lecithins and synthetic phospholipids can be used in the formulations. Other possible additives are mineral and vegetable oils.

If the extender used is water, it is also possible to use, for example, organic solvents as auxiliary solvents. Suitable liquid solvents are essentially: aromatic compounds, such as xylene, toluene or alkylnaphthalenes, chlorinated aromatic compounds or chlorinated aliphatic hydrocarbons, such as chlorobenzenes, chloroethylenes or methylene chloride, aliphatic hydrocarbons, such as cyclohexane or paraffins, for example mineral oil fractions, alcohols, such as butanol or glycol, and also ethers and esters thereof, ketones, such as acetone, methyl ethyl ketone, methyl isobutyl ketone or cyclohexanone, strongly polar solvents, such as dimethylformamide and dimethyl sulphoxide, and also water.

The compositions according to the invention may additionally comprise further components, such as, for example, surfactants. Suitable surfactants are emulsifiers and/or foam-formers, dispersants or wetting agents having ionic or nonionic properties, or mixtures of these surfactants. Examples of these are salts of polyacrylic acid, salts of lignosulphonic acid, salts of phenolsulphonic acid or naphthalenesulphonic acid, polycondensates of ethylene oxide with fatty alcohols or with fatty acids or with fatty amines, substituted phenols (preferably alkylphenols or arylphenols), salts of sulphosuccinic esters, taurine derivatives (preferably alkyl taurates), phosphoric esters of polyethoxylated alcohols or phenols, fatty esters of polyols, and derivatives of the compounds containing sulphates, sulphonates and phosphates, for example alkylaryl polyglycol ethers, alkylsulphonates, alkyl sulphates, arylsulphonates, protein hydrolysates, lignosulphite waste liquors and methylcellulose. The presence of a surfactant is required if one of the active compounds and/or one of the inert carriers is insoluble in water and the application is carried out in water. The proportion of surfactants is between 5 and 40 percent by weight of the compositions according to the invention.

It is possible to use colorants such as inorganic pigments, for example iron oxide, titanium oxide, Prussian blue, and organic dyes, such as alizarin dyes, azo dyes and metal phthalocyanine dyes, and trace nutrients, such as salts of iron, manganese, boron, copper, cobalt, molybdenum and zinc.

Other possible additives are perfumes, mineral or vegetable oils, if appropriate modified, waxes and nutrients (including trace nutrients), such as salts of iron, manganese, boron, copper, cobalt, molybdenum and zinc.

Stabilizers, such as low-temperature stabilizers, preservatives, antioxidants, light stabilizers or other agents which improve chemical and/or physical stability may also be present.

If appropriate, it is also possible for other addition components to be present, for example protective colloids, binders, glues, thickeners, thixotropic agents, penetrants, stabilizers, sequestrants, complex fomers. In general, the active compounds can be combined with any solid or liquid additive customarily used for formulation purposes.

The formulations generally comprise between 0.05 and 99% by weight, 0.01 and 98% by weight, preferably between 0.1 and 95% by weight, particularly preferably between 0.5 and 90% by weight, of active compound. very particularly preferably between 10 and 70 percent by weight.

The formulations described above can be employed in a method according to the invention for controlling unwanted microorganisms where the diaminopyrimidines according to the invention are applied to the microorganisms and/or their habitat.

The active compounds according to the invention, as such or in their formulations, can also be used in a mixture with known fungicides, bactericides, acaricides, nematicides or insecticides, for example to broaden the activity spectrum or to prevent the development of resistance.

Suitable mixing partners are, for example, known fungicides, insecticides, acaricides, nematicides or else bactericides (see also Pesticide Manual, 13th ed.).

A mixture with other known active compounds, such as herbicides, or with fertilizers and growth regulators, safeners and/or semiochemicals is also possible.

Application is carried out in a manner adapted to the use forms.

The control of phytopathogenic harmful fungi is primarily by treating the soil and the above-ground parts of the plants with crop protection compositions. Owing to concerns with a view to a possible impact of the crop protection compositions on the environment and human and animal health, there are efforts to reduce the amount of active compounds applied.

The active compounds can be applied as such, in the form of their formulations and the use forms prepared therefrom, such as ready-to-use solutions, suspensions, wettable powders, pastes, soluble powders, dusts and granules. Application is carried out in a customary manner, for example by watering, spraying, atomizing, broadcasting, dusting, foaming, painting-on, etc. It is also possible to apply the active compounds by the ultra-low-volume method or to inject the preparation of active compound or the active compound itself into the soil. It is also possible to treat the seed of the plants.

When using the active compounds according to the invention as fungicides, the application rates can be varied within a relatively wide range, depending on the type of application. The application rate of the active compounds according to the invention is
in the treatment of parts of plants, for example leaves: from 0.1 to 10 000 g/ha, preferably from 10 to 1000 g/ha, particularly preferably from 50 to 300 g/ha (when the application is by watering or dripping, it is possible to reduce the application rate even more, in particular when inert substrates such as rock wool or perlite are used);
in the treatment of seed: from 2 to 200 g per 100 kg of seed, preferably from 3 to 150 g per 100 kg of seed, particularly preferably from 2.5 to 25 g per 100 kg of seed, very particularly preferably from 2.5 to 12.5 g per 100 kg of seed;
in soil treatment: from 0.1 to 10 000 g/ha, preferably from 1 to 5000 g/ha.

These application rates are mentioned only in an exemplary manner and not limiting for the purpose of the invention.

The compounds according to the invention can also be used for protecting objects which come into contact with salt water or brackish water, such as hulls, screens, nets, buildings, moorings and signalling systems, against colonization.

The active compounds according to the invention, alone or in combination with other active compounds, can furthermore be employed as antifouling agents.

The treatment method according to the invention can be used for treating genetically modified organisms (GMOs), for example plants or seeds. Genetically modified plants (or transgenic plants) are plants in which a heterologous gene has been stably integrated into the genome. The expression “heterologous gene” essentially means a gene which is provided or assembled outside the plant and when introduced in the nuclear, chloroplastic or mitochondrial genome gives the transformed plant new or improved agronomic or other properties by expressing a protein or polypeptide of interest or by downregulating or silencing other gene(s) which is/are present in the plant (using for example, antisense technology, cosuppression technology or RNA interference—RNAi—technology). A heterologous gene that is located in the genome is also called a transgene. A transgene that is defined by its particular location in the plant genome is called a transformation or transgenic event.

Depending on the plant species or plant cultivars, their location and growth conditions (soils, climate, vegetation period, diet), the treatment according to the invention may also result in superadditive (“synergistic”) effects. Thus, for example, the following effects, which exceed the effects which were actually to be expected, are possible: reduced application rates and/or a widening of the activity spectrum and/or an increase in the activity of the active compounds and compositions which can be used according to the invention, better plant growth, increased tolerance to high or low temperatures, increased tolerance to drought or to water or soil salt content, increased flowering performance, easier harvesting, accelerated maturation, higher harvest yields, bigger fruits, larger plant height, greener leaf colour, earlier flowering, higher quality and/or a higher nutritional value of the harvested products, higher sugar concentration within the fruits, better storage stability and/or processability of the harvested products.

In the present case, unwanted phytopathogenic fungi and/or microorganisms and/or viruses are to be understood as meaning phytopathogenic fungi, bacteria and viruses. Thus, the substances according to the invention can be employed for protecting plants against attack by the abovementioned pathogens within a certain period of time after the treatment. The period of time within which protection is effected generally extends from 1 to 10 days, preferably 1 to 7 days, after the treatment of the plants with the active compounds.

Plants and plant cultivars which are preferably treated according to the invention include all plants with genetic material which bestows upon these plants particularly advantageous useful properties (whether this was achieved by breeding and/or biotechnology is immaterial).

Plants and plant cultivars which are also preferably treated according to the invention are resistant against one or more biotic stresses, i.e. said plants have a better defence against animal and microbial pests, such as against nematodes, insects, mites, phytopathogenic fungi, bacteria, viruses and/or viroids.

Plants and plant cultivars which may also be treated according to the invention are those plants which are resistant to one or more abiotic stress factors. Abiotic stress conditions may include, for example, drought, cold temperature exposure, heat exposure, osmotic stress, flooding, increased soil salinity, increased mineral exposure, ozone exposure, high light exposure, limited availability of nitrogen nutrients, limited availability of phosphorus nutrients or shade avoidance.

Plants and plant cultivars which may also be treated according to the invention are those plants characterized by enhanced yield characteristics. Increased yield in said plants can be the result of, for example, improved plant physiology, growth and development, such as water use efficiency, water retention efficiency, improved nitrogen use, enhanced carbon assimilation, improved photosynthesis, increased germination efficiency and accelerated maturation. Yield can furthermore by affected by improved plant architecture (under stress and non-stress conditions), including early flowering, flowering control for hybrid seed production, seedling vigour, plant size, internode number and distance, root growth, seed size, fruit size, pod size, pod or ear number, seed number per pod or ear, seed mass, enhanced seed filling, reduced seed dispersal, reduced pod dehiscence and lodging resistance. Further yield traits include seed composition, such as carbohydrate content, protein content, oil content and composition, nutritional value, reduction in anti-nutritional compounds, improved processability and better storage stability.

Plants that may be treated according to the invention are hybrid plants that already express the characteristics of heterosis or the hybrid effect which results in generally higher yield, vigour, health and resistance towards biotic and abiotic stress factors. Such plants are typically made by crossing an inbred male sterile parent line (the female parent) with another inbred male fertile parent line (the male parent). Hybrid seed is typically harvested from the male sterile plants and sold to growers. Male sterile plants can sometimes (e.g. in corn) be produced by detasseling, (i.e. the mechanical removal of the male reproductive organs or male flowers) but, more typically, male sterility is the result of genetic determinants in the plant genome. In that case, and especially when seed is the desired product to be harvested from the hybrid plants, it is typically useful to ensure that male fertility in the hybrid plants, which contain the genetic determinants responsible for male sterility, is fully restored. This can be accomplished by ensuring that the male parents have appropriate fertility restorer genes which are capable of restoring the male fertility in hybrid plants that contain the genetic determinants responsible for male sterility. Genetic determinants for male sterility may be located in the cytoplasm. Examples of cytoplasmic male sterility (CMS) were for instance described in Brassica species. However, genetic determinants for male sterility can also be located in the nuclear genome. Male sterile plants can also be obtained by plant biotechnology methods such as genetic engineering. A particularly useful means of obtaining male sterile plants is described in WO 89/10396 in which, for example, a ribonuclease such as a barnase is selectively expressed in the tapetum cells in the stamens. Fertility can then be restored by expression in the tapetum cells of a ribonuclease inhibitor such as barstar.

Plants or plant cultivars (obtained by plant biotechnology methods such as genetic engineering) which may be treated according to the invention are herbicide-tolerant plants, i.e. plants made tolerant to one or more given herbicides. Such plants can be obtained either by genetic transformation, or by selection of plants containing a mutation imparting such herbicide tolerance.

Herbicide-tolerant plants are for example glyphosate-tolerant plants, i.e. plants made tolerant to the herbicide glyphosate or salts thereof. For example, glyphosate-tolerant plants can be obtained by transforming the plant with a gene encoding the enzyme 5-enolpyruvylshikimate-3-phosphate synthase (EPSPS). Examples of such EPSPS genes are the AroA gene (mutant CT7) of the bacterium Salmonella typhimurium, the CP4 gene of the bacterium Agrobacterium sp., the genes encoding a petunia EPSPS, a tomato EPSPS, or an Eleusine EPSPS. It can also be a mutated EPSPS. Glyphosate-tolerant plants can also be obtained by expressing a gene that encodes a glyphosate oxidoreductase enzyme. Glyphosate-tolerant plants can also be obtained by expressing a gene that encodes a glyphosate acetyl transferase enzyme. Glyphosate-tolerant plants can also be obtained by selecting plants containing naturally-occurring mutations of the above-mentioned genes.

Other herbicide-resistant plants are for example plants that are made tolerant to herbicides inhibiting the enzyme glutamine synthase, such as bialaphos, phosphinothricin or glufosinate. Such plants can be obtained by expressing an enzyme detoxifying the herbicide or a mutant glutamine synthase enzyme that is resistant to inhibition. One such efficient detoxifying enzyme is, for example, an enzyme encoding a phosphinothricin acetyltransferase (such as the bar or pat protein from Streptomyces species). Plants expressing an exogenous phosphinothricin acetyltransferase have been described.

Further herbicide-tolerant plants are also plants that are made tolerant to the herbicides inhibiting the enzyme hydroxyphenylpyruvatedioxygenase (HPPD). Hydroxyphenylpyruvatedioxygenases are enzymes that catalyse the reaction in which para-hydroxyphenylpyruvate (HPP) is transformed into homogentisate. Plants tolerant to HPPD-inhibitors can be transformed with a gene encoding a naturally-occurring resistant HPPD enzyme, or a gene encoding a mutated HPPD enzyme. Tolerance to HPPD-inhibitors can also be obtained by transforming plants with genes encoding certain enzymes enabling the formation of homogentisate despite the inhibition of the native HPPD enzyme by the HPPD-inhibitor. Tolerance of plants to HPPD inhibitors can also be improved by transforming plants with a gene encoding an enzyme prephenate dehydrogenase in addition to a gene encoding an HPPD-tolerant enzyme.

Still further herbicide-resistant plants are plants that are made tolerant to acetolactate synthase (ALS) inhibitors. Known ALS-inhibitors include, for example, sulphonylurea, imidazolinone, triazolopyrimidmes, pyrimidinyloxy(thio)benzoates, and/or sulphonylaminocarbonyltriazolinone herbicides. Different mutations in the ALS enzyme (also known as acetohydroxyacid synthase, AHAS) are known to confer tolerance to different herbicides and groups of herbicides. The production of sulphonylurea-tolerant plants and imidazolinone-tolerant plants has been described in the international publication WO 1996/033270. Further sulphonylurea- and imidazolinone-tolerant plants have also been described, for example in WO 2007/024782.

Other plants tolerant to imidazolinone and/or sulphonylurea can be obtained by induced mutagenesis, by selection in cell cultures in the presence of the herbicide or by mutation breeding.

Plants or plant cultivars (obtained by plant biotechnology methods such as genetic engineering) which may also be treated according to the invention are insect-resistant transgenic plants, i.e. plants made resistant to attack by certain target insects. Such plants can be obtained by genetic transformation, or by selection of plants containing a mutation imparting such insect resistance.

In the present context, the term “insect-resistant transgenic plant” includes any plant containing at least one transgene comprising a coding sequence encoding:
1) an insecticidal crystal protein from Bacillus thuringiensis or an insecticidal portion thereof, such as the insecticidal crystal proteins listed online at: http://www.lifesci.sussex.ac.uk/Home/Neil_Crickmore/Bt/, or insecticidal portions thereof, for example proteins of the Cry protein classes Cry1Ab, Cry1Ac, Cry1F, Cry2Ab, Cry3Ae or Cry3Bb or insecticidal portions thereof; or
2) a crystal protein from Bacillus thuringiensis or a portion thereof which is insecticidal in the presence of a second other crystal protein from Bacillus thuringiensis or a portion thereof, such as the binary toxin made up of the Cy34 and Cy35 crystal proteins; or
3) a hybrid insecticidal protein comprising parts of two different insecticidal crystal proteins from Bacillus thuringiensis, such as a hybrid of the proteins of 1) above or a hybrid of the proteins of 2) above, for example the Cry1A.105 protein produced by maize event MON98034 (WO 2007/027777); or
4) a protein of any one of 1) to 3) above wherein some, particularly 1 to 10, amino acids have been replaced by another amino acid to obtain a higher insecticidal activity to a target insect species, and/or to expand the range of target insect species affected, and/or because of changes induced in the encoding DNA during cloning or transformation, such as the Cry3Bb1 protein in maize events MON863 or MON88017, or the Cry3A protein in maize event MIR604;
5) an insecticidal secreted protein from Bacillus thuringiensis or Bacillus cereus, or an insecticidal portion thereof, such as the vegetative insecticidal proteins (VIP) listed at: http://www.lifesci.sussex.ac.uk/home/Neil_Crickmore/Bt/vip.html, for example proteins from the VIP3Aa protein class; or
6) a secreted protein from Bacillus thuringiensis or Bacillus cereus which is insecticidal in the presence of a second secreted protein from Bacillus thuringiensis or B. cereus, such as the binary toxin made up of the VIP1a and VIP2A proteins;
7) a hybrid insecticidal protein comprising parts from different secreted proteins from Bacillus thuringiensis or Bacillus cereus, such as a hybrid of the proteins in 1) above or a hybrid of the proteins in 2) above; or
8) a protein of any one of 1) to 3) above wherein some, particularly 1 to 10, amino acids have been replaced by another amino acid to obtain a higher insecticidal activity to a target insect species, and/or to expand the range of target insect species affected, and/or because of changes induced in the encoding DNA during cloning or transformation (while still encoding an insecticidal protein), such as the VIP3Aa protein in cotton event COT102.

Of course, insect-resistant transgenic plants, as used herein, also include any plant comprising a combination of genes encoding the proteins of any one of the above classes 1 to 8. In one embodiment, an insect-resistant plant contains more than one transgene encoding a protein of any one of the above classes 1 to 8, to expand the range of target insect species affected or to delay insect resistance development to the plants, by using different proteins insecticidal to the same target insect species but having a different mode of action, such as binding to different receptor binding sites in the insect.

Plants or plant cultivars (obtained by plant biotechnology methods such as genetic engineering) which may also be treated according to the invention are tolerant to abiotic stresses. Such plants can be obtained by genetic transformation, or by selection of plants containing a mutation imparting such stress resistance. Particularly useful stress tolerance plants include:
a. plants which contain a transgene capable of reducing the expression and/or the activity of the poly(ADP-ribose)polymerase (PARP) gene in the plant cells or plants.
b. plants which contain a stress tolerance-enhancing transgene capable of reducing the expression and/or the activity of the PARG encoding genes of the plants or plant cells;
c. plants which contain a stress tolerance-enhancing transgene coding for a plant-functional enzyme of the nicotinamide adenine dinucleotide salvage biosynthesis pathway, including nicotinamidase, nicotinate phosphoribosyltransferase, nicotinic acid mononucleotide adenyl transferase, nicotinamide adenine dinucleotide synthetase or nicotinamide phosphoribosyltransferase.

Plants or plant cultivars (obtained by plant biotechnology methods such as genetic engineering) which may also be treated according to the invention show altered quantity, quality and/or storage-stability of the harvested product and/or altered properties of specific ingredients of the harvested product such as, for example:
1) transgenic plants which synthesize a modified starch, which in its physical-chemical characteristics, in particular the amylose content or the amylose/amylopectin ratio, the degree of branching, the average chain length, the side chain distribution, the viscosity behaviour, the gelling strength, the starch grain size and/or the starch grain morphology, is changed in comparison with the synthesized starch in wild type plant cells or plants, so that this modified starch is better suited for special applications.
2) transgenic plants which synthesize non-starch carbohydrate polymers or which synthesize non-starch carbohydrate polymers with altered properties in comparison to wild type plants without genetic modification. Examples are plants which produce polyfructose, especially of the inulin and levan type, plants which produce alpha-1,4-glucans, plants which produce alpha-1,6 branched alpha-1,4-glucans, and plants producing alternan.
3) transgenic plants which produce hyaluronan.

Plants or plant cultivars (that can be obtained by plant biotechnology methods such as genetic engineering) which may also be treated according to the invention are plants, such as cotton plants, with altered fibre characteristics. Such plants can be obtained by genetic transformation, or by selection of plants containing a mutation imparting such altered fibre characteristics and include:
a) plants, such as cotton plants, which contain an altered form of cellulose synthase genes,
b) plants, such as cotton plants, which contain an altered form of rsw2 or rsw3 homologous nucleic acids;
c) plants, such as cotton plants, with an increased expression of sucrose phosphate synthase;
d) plants, such as cotton plants, with an increased expression of sucrose synthase;
e) plants, such as cotton plants, wherein the timing of the plasmodesmatal gating at the basis of the fibre cell is altered, for example through downregulation of fibre-selective β-1,3-glucanase;
f) plants, such as cotton plants, which have fibres with altered reactivity, for example through the expression of the N-acetylglucosaminetransferase gene including nodC and chitin synthase genes.

Plants or plant cultivars (that can be obtained by plant biotechnology methods such as genetic engineering) which may also be treated according to the invention are plants, such as oilseed rape or related Brassica plants, with altered oil profile characteristics. Such plants can be obtained by genetic transformation or by selection of plants containing a mutation imparting such altered oil characteristics and include:
a) plants, such as oilseed rape plants, which produce oil having a high oleic acid content;
b) plants, such as oilseed rape plants, which produce oil having a low linolenic acid content;
c) plants, such as oilseed rape plants, which produce oil having a low level of saturated fatty acids.

Particularly useful transgenic plants which may be treated according to the invention are plants which comprise one or more genes which encode one or more toxins, are the following which are sold under the trade names YIELD GARD® (for example maize, cotton, soya beans), KnockOut® (for example maize), BiteGard® (for example maize), Bt-Xtra® (for example maize), StarLink® (for example maize), Bollgard® (cotton), Nucotn® (cotton), Nucotn 33B® (cotton), NatureGard® (for example maize), Protecta® and NewLeaf® (potato). Examples of herbicide-tolerant plants which may be mentioned are maize varieties, cotton varieties and soya bean varieties which are sold under the trade names Roundup Ready® (tolerance to glyphosate, for example maize, cotton, soya beans), Liberty Link® (tolerance to phosphinothricin, for example oilseed rape), IMI® (tolerance to imidazolinone) and SCS® (tolerance to sulphonylurea, for example maize). Herbicide-resistant plants (plants bred in a conventional manner for herbicide tolerance) which may be mentioned include the varieties sold under the name Clearfield® (for example maize).

Particularly useful transgenic plants which may be treated according to the invention are plants containing transformation events, or a combination of transformation events, that are listed for example in the databases for various national or regional regulatory agencies (see for example http://gmoinfojrc.it/gmp_browse.aspx and http://www.agbios.com/dbase.php).

According to the invention, the plants listed can be treated particularly advantageously with the compounds of the general formula (I) or the active compound mixtures according to the invention. The preferred ranges indicated above for the active compounds and mixtures also apply to the treatment of these plants. Particular emphasis is given to treating the plants with the compounds and mixtures specifically indicated in the present text.

The compositions or active compounds according to the invention can also be used to protect plants for a certain period after treatment against attack by the pathogens mentioned. The period for which protection is provided generally extends over 1 to 28 days, preferably over 1 to 14 days, particularly preferably over 1 to 10 days, very particularly preferably over 1 to 7 days, after the treatment of the plants with the active compounds, or over up to 200 days after seed treatment.

Preparation and use of the active compounds of the formula (I) according to the invention is shown in the examples below. However, the invention is not limited to these examples.

### Preparation of Starting Materials of the Formula (V)

### 2,5-Dichloro-N-(3-methoxypropan-2-yl)pyrimidine-4-amine (V-1)

At −10° C., 5.42 g (39.3 mmol) of potassium carbonate are added to a solution of 6.00 g (32.7 mmol) of 2,4,5-trichloropyrimidine in 100 ml of acetonitrile. 3.06 g (34.4 mmol) of 2-amino-1-methoxypropane are then added dropwise as a 20% strength solution in acetonitrile. With stirring, the reaction mixture is allowed to warm to room temperature. After 16 h, the reaction mixture is stirred into 250 ml of ice-water/dilute hydrochloric acid (1:1). The precipitate formed is filtered off and dried. This gives 5.10 g (64%) of 2,5-dichloro-N-(3-methoxypropan-2-yl)pyrimidine-4-amin (V-1) (logP (pH 2.3): 2.10). ¹H NMR (400 MHz, MeCN-d) δ=8.02 (s, 1H), 6.03 (br. s, 1H), 4.39-4.33 (m, 1H), 3.48-3.40 (m, 2H), 3.33 (s, 3H), 1.23 (d, 3H).

The following compounds can be prepared in an analogous manner:

5-bromo-2-chloro-N-(3-methoxypropan-2-yl)pyrimidine-4-amine (V-2) (logP (pH 2.3): 2.26). ¹H NMR (400 MHz, DMSO-d) δ=8.22 (s, 1H), 6.98 (br. d, 1H), 4.36 (br. m, 1H), 3.48 (dd, 1H), 3.36 (dd, 1H), 3.28 (s, 1H), 1.17 (d, 3H).

2,5-dichloro-N-(2-methoxyethyl)pyrimidine-4-amine (V-3) (logP (pH 2.3): 1.65). ¹H NMR (400 MHz, MeCN-d) δ=8.02 (s, 1H), 6.23 (br. s, 1H), 3.63−3.59 (m, 2H), 3.55-3.5 (m, 2H), 3.33 (s, 3H).

2,5-dichloro-N-(1-hydroxybutan-2-yl)pyrimidine-4-amine (V-4) (logP (pH 2.3): 1.65). ¹H NMR (400 MHz, MeCN-d) δ=8.02 (s, 1H), 5.99 (br. s, 1H), 4.14-4.06 (m, 1H), 3.62-3.59 (m, 2H), 2.83-2.80 (m, 1H), 1.72-1.54 (m, 2H), 0.94-0.91 (m, 3H).

2,5-dichloro-N-(2-hydroxyethyl)pyrimidine-4-amine (V-5) (logP (pH 2.3): 0.97). ¹H NMR (400 MHz, MeCN-d) δ=8.02 (s, 1H), 6.32 (br. s, 1H), 3.69-3.66 (m, 2H), 3.57-3.53 (m, 2H).

2,5-dichloro-N-[1-(methylsulphanyl)propan-2-yl]pyrimidine-4-amine (V-6) (logP (pH 2.3): 2.73). ¹H NMR (400 MHz, MeCN-d) δ=8.03 (s, 1H), 6.07 (br. s, 1H), 4.42-4.35 (m, 1H), 2.79-2.66 (m, 2H), 2.12 (s, 3H), 1.32 (d, 3H).

2,5-dichloro-N-[2-methyl-1-(methylsulphanyl)propan-2-yl]pyrimidine-4-amine (V-7) (logP (pH 2.3): 3.47). ¹H NMR (400 MHz, MeCN-d) δ=8.03 (s, 1H), 5.89 (br. s, 1H), 3.09 (s, 2H), 2.09 (s, 3H), 1.53 (s, 6H).

[(2,5-dichloropyrimidin-4-yl)amino]acetonitrile (V-8) (logP (pH 2.3): 1.27). ¹H NMR (400 MHz, MeCN-d) δ=8.16 (s, 1H), 6.70 (br. s, 1H), 4.35 (d, 2H).

2,5-dichloro-N-(propan-2-yl)pyrimidine-4-amine (V-9) (logP (pH 2.3): 2.46). ¹H NMR (400 MHz, MeCN-d) δ=7.99 (s, 1H), 5.92 (hr. s, 1H), 4.31-4.23 (m, 1H), 1.25 (d, 6H).

5-bromo-2-chloro-N-(propan-2-yl)pyrimidine-4-amine (V-10) (logP (pH 2.3): 2.62). ¹H NMR (400 MHz, ACETONITRILE-d) δ=8.19 (s, 1H), 7.03 br. s, 1H), 4.25 (br. m, 1H), 1.22 (d, 6H).

2,5-dichloro-N-methyl-N-(propan-2-yl)pyrimidine-4-amine (V-11) (logP (pH 2.3): 3.16). ¹H NMR (400 MHz, MeCN-d) δ=8.04 (s, 1H), 4.82-4.76 (m, 1H), 3.03 (s, 3H), 1.22 (d, 6H).

2,5-dichloro-N-(cyclopentyl)pyrimidine-4-amine (V-12) (logP (pH 2.3): 3.16). ¹H NMR (400 MHz, DMSO-d) δ=8.11-8.09 (d, 1H), 7.36 (d, 1H), 4.36-4.28 (m, 1H), 1.98-1.93 (m, 2H), 1.73-1.67 (m, 2H), 1.64-1.53 (m, 4H).

2,5-dichloro-N-(prop-2-en-1-yl)pyrimidine-4-amine (V-13) (logP (pH 2.3): 2.12). ¹H NMR (400 MHz, MeCN-d) δ=8.03 (s, 1H), 6.40 (br. s, 1H), 5.98-5.88 (m, 1H), 5.23-5.12 (m, 2H), 4.09-4.06 (m, 2H).

2,5-dichloro-N-(butan-2-yl)pyrimidine-4-amine (V-14) (logP (pH 2.3): 2.94). ¹H NMR (400 MHz, MeCN-d) δ=7.99 (s, 1H), 5.90 (br. s, 1H), 4.15-4.08 (m, 1H), 1.67-1.56 (m, 2H), 1.21 (d, 3 H), 0.91 (t, 3H).

N-(butan-2-yl)-2,5-dichloro-N-methylpyrimidine-4-amine (V-15) (logP (pH 2.3): 3.62). ¹H NMR (400 MHz, MeCN-d) δ=8.04 (s, 1H), 4.62-4.56 (m, 1H), 3.01 (s, 3H), 1.70-1.52 (m, 2H), 1.21 (d, 3H), 0.84 (t, 3H).

N-(butan-1-yl)-2,5-dichloropyrimidine-4-amine (V-16) (logP (pH 2.3): 2.91). ¹H NMR (400 MHz, MeCN-d) δ=7.99 (s, 1H), 6.23 (br. s, 1H), 3.47-3.42 (m, 2H), 1.63-1.56 (m, 2H), 1.42-1.33 (m, 2H), 0.92 (t, 3H).

N-t-butyl-2,5-dichloropyrimidine-4-amine (V-17) (logP (pH 2.3): 3.22). ¹H NMR (400 MHz, MeCN-d) δ=8.00 (s, 1H), 5.73 (br. s, 1H), 1.48 (s, 9 H.

2,5-dichloro-N-cyclohexylpyrimidine-4-amine (V-18) (logP (pH 2.3): 3.47). ¹H NMR (400 MHz, MeCN-d) δ=7.99 (s, 1H), 5.91 (br. s, 1H), 3.99-3.91 (m, 1H), 1.79-1.20 (m, 10H).

2,5-dichloro-N-ethyl-N-methylpyrimidine-4-amine (V-19) (logP (pH 2.3): 2.68). ¹H NMR (400 MHz, DMSO-d) δ=8.14 (s, 1H), 3.67 (q, 2H), 3.18 (s, 3H), 1.19 (t, 3H).

2,5-dichloro-N-ethylpyrimidine-4-amine (V-20) (logP (pH 2.3): 1.93). ¹H NMR (400 MHz, MeCN-d) δ=7.99 (s, 1H), 6.23 (br. s, 1H), 3.48 (q, 2H), 1.20 (t, 3H).

2,5-dichloro-N-propylpyrimidine-4-amine (V-21): logP (pH 2.3): 2.42). ¹H NMR (400 MHz, DMSO-d) δ=8.14 (s, 1H), 7.94 (br. s, 1H), 3.30 (t, 2H), 1.58-1.53 (m, 2H), 0.87 (t, 3H).

2,5-dichloro-N-(3-methylbutyl)pyrimidine-4-amine (V-22): logP (pH 2.3): 3.45). ¹H NMR (400 MHz, DMSO-d) δ=8.08 (s, 1H), 7.67 (br. s, 1H), 3.42-3.37 (m, 2H), 1.63-1.56 (m, 1H), 1.49-1.44 (m, 2H), 0.89 (d, 6H).

2,5-dichloro-N-(2-ethylpropyl)pyrimidine-4-amine (V-23): logP (pH 2.3): 3.39). ¹H NMR (400 MHz, DMSO-d) δ=8.09 (s, 1H), 7.17 (br. s, 1H), 3.99-3.93 (m, 1H), 1.62-1.54 (m, 4H), 0.84 (t, 6H).

2,5-dichloro-N-(2-methylbutyl)pyrimidine-4-amine (V-24): logP (pH 2.3): 3.42). ¹H NMR (400 MHz, DMSO-d) δ=8.09 (s, 1H), 7.63 (br. s, 1H), 3.35-3.29 (m, 1H), 3.23-3.11 (m, 1H), 1.77-1.73 (m, 1H), 1.41-1.35 (m, 1H), 1.17-1.10 (m, 1H), 0.90-85 (m, 6H).

2,5-dichloro-N-methyl-N-propylpyrimidine-4-amine (V-25): logP (pH 2.3): 3.31). ¹H NMR (400 MHz, DMSO-d) δ=8.14 (s, 1H), 3.61 (t, 2H), 3.22 (s, 3H), 1.69-1.60 (m, 2H), 0.87 (t, 3H).

2,5-dichloro-N-methyl-N-(2-methylpropyl)pyrimidine-4-amine (V-26): logP (pH 2.3): 3.78). ¹H NMR (400 MHz, DMSO-d) δ=8.14 (s, 1H), 3.55 (d, 2H), 3.23 (s, 3H), 2.07-2.00 (m, 1H), 0.86 (d, 6H).

2,5-dichloro-N-(pent-2-yl)pyrimidine-4-amine (V-27): logP (pH 2.3): 3.47). ¹H NMR (400 MHz, DMSO-d) δ=8.09 (s, 1H), 7.26 (br.s, 1H), 4.22-4.14 (m, 1H), 1.67-1.58 (m, 1H), 1.52-1.44 (m, 1H), 1.34-1.18 (m, 2H), 1.12 (d, 3H), 0.88 (t, 3H).

2,5-dichloro-N-methyl-N-propylpyrimidine-4-amine (V-28): logP (pH 2.3): 3.89). ¹H NMR (400 MHz, DMSO-d) δ=8.13 (s, 1H), 3.64 (t, 2H), 3.20 (s, 3H), 1.64-1.57 (m, 2H), 1.35-1.29 (m, 2H), 0.90 (t, 3H).

2,5-dichloro-N-(2-methylpropyl)pyrimidine-4-amine (V-29): logP (pH 2.3): 2.93). ¹H NMR (400 MHz, DMSO-d) δ=8.10 (s, 1H), 7.66 (br.s, 1H), 3.21 (t, 2H), 1.99-1.92 (m, 1H), 0.88 (d, 6 H).

2,5-dichloro-N-ethyl-N-isopropylpyrimidine-4-amine (V-30): logP (pH 2.3): 3.80). ¹H NMR (400 MHz, DMSO-d) δ=8.16 (s, 1H), 4.77-4.70 (m, 1H), 3.58 (q, 2H), 1.23 (d, 6H), 1.14 (t, 3H).

2,5-dichloro-N-(pentyl)pyrimidine-4-amine (V-31): logP (pH 2.3): 3.51). ¹H NMR (400 MHz, DMSO-d) δ=8.08 (s, 1H), 7.67 (br.s, 1H), 3.39-3.34 (m, 2H), 1.61-1.54 (m, 2H), 1.36-1.24 (m, 4H), 0.88 (t, 3H).

2,5-dichloro-N-(2,2-dimethylpropyl)pyrimidine-4-amine (V-32): logP (pH 2.3): 3.44). ¹H NMR (400 MHz, DMSO-d) δ=8.12 (s, 1H), 7.39 (br.s, 1H), 3.28 (d, 2H), 0.90 (s, 9H).

2,5-dichloro-N-methyl-N-(prop-2-en-1-yl)pyrimidine-4-amine (V-33) (logP (pH 2.3). 2.98 ¹H NMR (400 MHz, DMSO-d) δ=8.22 (s, 1H), 5.93-5.85 (m, 1H), 5.23-5.20 (m, 2H), 4.27-4.25 (m, 2H), 3.15 (s, 3H).

### 2-Chloro-N-(3-methoxypropan-2-yl)-5-trifluoromethylpyrimidine-4-amine (V-34)

At −10° C., 1.91 g (13.8 mmol) of potassium carbonate are added to a solution of 2.00 g (9.22 mmol) of 2,4-dichloro-5-(trifluoromethyl)pyrimidine in 80 ml of acetonitrile. 0.86 g (9.68 mmol) of 2-amino-1-methoxypropane is then added dropwise as a 30% strength solution in acetonitrile. With stirring, the reaction mixture is allowed to warm to room temperature. After 16 h, the reaction mixture is stirred into 250 ml of ice-water and extracted with dichloromethane (3×100 ml). The combined organic phases are separated off, washed with water (2×100 ml), dried over MgSO₄ and freed from the solvent under reduced pressure. The crude product is purified by column chromatography on silica gel (cyclohexane/ethyl acetate). This gives 0.75 g (26%) of 2-chloro-N-(3-methoxypropan-2-yl)-5-trifluoromethylpyrimidine-4-amine (V-21) (logP (pH 2.3): 2.75). ¹H NMR (400 MHz, DMSO-d) δ=8.28 (s, 1H), 3.56-3.52 (m, 3H), 3.33-3.32 (d, 3H), 1.24-1.22 (q, 3H).

### Preparation of Compounds of the Formula (Iv)

### N-Ethyl-N-methyl-1-(3-nitrophenyl)methanesulphonamide (IV-1)

At 5° C., 5.00 g (84.8 mmol) of ethylmethylamine, as a 40% strength solution in ethyl acetate, is added dropwise over a period of 30 min to a solution of 5.00 g (21.2 mmol) of (3-nitrophenyl)methanesulphonyl chloride in 100 ml of ethyl acetate. The mixture is then stirred at 60° C. for 3 h. After cooling, the reaction mixture is stirred into 250 ml of ice-water/dil. hydrochloric acid (1:1) and extracted with ethyl acetate (2×100 ml). The combined organic phases are washed with 100 ml of water, dried over MgSO₄ and freed from the solvent under reduced pressure. This gives 5.03 g (90%) of the desired product (logP (pH 2.3): 2.05). ¹H NMR (400 MHz, DMSO-d) δ=8.29 (s, 1H), 8.20 (d, 1H), 7.85 (d, 1H), 7.68 (dd, 1H), 4.57 (s, 2H), 3.11 (q, 2H), 2.71 (s, 3H), 1.05 (t, 3H).

The following compounds can be prepared in an analogous manner:

N-methyl-N-(propan-2-yl)-1-(3-nitrophenyl)methanesulphonamide (IV-2) (logP (pH 2.3): 2.35). ¹H NMR (400 MHz, DMSO-d) δ=8.29 (s, 1H), 8.20 (d, 1H), 7.85 (d, 1H), 7.67 (dd, 1H), 4.55 (s, 2H), 3.93 (m, 1H), 2.63 (s, 3H), 1.05 (d, 6H).

N-(1-phenylethyl)-1-(3-nitrophenyl)methanesulphonamide (IV-3) logP (pH 2.3): 2.63). ¹H NMR (400 MHz, MeCN-d) δ=8.18 (d, 1H), 8.06 (s, 1H), 7.62 (d, 1H), 7.55 (dd, 1H), 7.39-7.27 (m, 5H) 5.83 (d, 1H), 4.52 (m, 1H), 4.21 (m, 2H), 1.43 (d, 3H).

N-(benzyl)-1-(3-nitrophenyl)methanesulphonamide (IV-4) logP (pH 2.3): 2.43). ¹H NMR (400 MHz, MeCN-d) δ=8.19 (d, 1H), 8.17 (s, 1H), 7.72 (d, 1H), 7.61 (dd, 1H), 7.37-7.26 (m, 5H) 4.35 (s, 2H), 4.19 (d, 2H).

### 1-(3-Aminophenyl)-N-ethyl-N-methylmethanesulphonamide (IV-5)

In an autoclave, 4.50 g (17.4 mmol) of N-ethyl-N-methyl-1-(3-nitrophenyl)methanesulphonamide and 1 g of Pd/C (10%) are initially charged in 100 ml of methanol, and a hydrogen pressure of 5 bar is applied at 30° C. After 10 h, the catalyst is filtered off and the filtrate is concentrated under reduced pressure. This gives 3.45 g (87%) of the desired product (logP (pH 2.3): 0.71). ¹H NMR (400 MHz, DMSO-d) δ=6.98 (dd, 1H), 6.60 (s, 1H), 6.55-6.51 (m, 2H), 4.93 (br. s, 2H), 4.13 (s, 2H), 3.04 (q, 2H), 2.68 (s, 3H), 1.03 (t, 3H).

The following compounds can be prepared in an analogous manner:

1-(3-aminophenyl)-N-methyl-N-(propan-2-yl)methanesulphonamide (IV-6) (logP (pH 2.3): 1.08). ¹H NMR (400 MHz, DMSO-d) δ=6.98 (dd, 1H), 6.59 (s, 1H), 6.53-6.50 (m, 2H), 5.01 (br. s, 2H), 4.14 (s, 2H), 3.90 (m, 1H), 2.59 (s, 3H), 1.02 (d, 6H).

1-(3-aminophenyl)-N-(1-phenylethyl)methanesulphonamide (IV-7) (logP (pH 2.3): 1.53). ¹H NMR (400 MHz, DMSO-d) δ=7.42-7.21 (m, 6H) 6.93 (dd, 1H), 6.53-6.49 (m, 2H), 6.38 (d, 4.86 (br. s, 2H), 4.43 (m, 1H), 3.94-3.84 (m, 2H), 1.37 (d, 3H).

1-(3-aminophenyl)-N-benzylmethanesulphonamide (IV-8) (logP (pH 2.3): 1.32). ¹H NMR (400 MHz, DMSO-d) δ=7.38-7.22 (m, 6H) 6.98 (dd, 1H), 6.58 (s, 1H), 6.55 (d, 1H), 6.49 (d, 1H), 4.91 (br. s, 2H), 4.08 (s, 2H), 4.06 (d, 2H).

N-(3-aminophenyl)-N-methylpropanamide (IV-9) (logP (pH 2.3): 0.63). ¹H NMR (400 MHz, DMSO-d) δ=7.04 (dd, 1H) 6.53 (dd, 1H), 6.44 (dd, 1H), 6.36 (dd, 1H), 5.07 (br.s, 2H), 3.08 (s, 3 H), 2.06 (q, 2H), 0.93 (t, 3H).

### 1-(3-Aminobenzyl)-3-methylimidazolidin-2-one (IV-10)

At 0° C., 0.96 g (24.1 mmol) of sodium hydride (60% in paraffin) is added a little at a time to a solution of 2.04 g (20.4 mmol) of 1-methylimidazolidin-2-one in 50 ml of tetrahydrofuran, and the reaction mixture is stirred for 30 min. A solution of 4.00 g (18.5 mmol) of 3-nitrobenzyl bromide in tetrahydrofuran is added dropwise to the solution, and the mixture is allowed to warm to room temperature. After 3 h, the reaction mixture is stirred into 250 ml of ice-water/dil. hydrochloric acid (1:1) and extracted with ethyl acetate (2×100 ml). The combined organic phases are washed with 100 ml of water, dried over MgSO₄, filtered through silica gel and freed from the solvent under reduced pressure. This gives 3.56 g (81%) of 1-methyl-3-(3-nitrobenzyl)imidazolidin-2-one (logP (pH 2.3): 2.03), ¹H NMR (400 MHz, DMSO-d) δ=8.12 (d, 1H), 8.07 (s, 1H), 7.70 (d, 1H), 7.63 (dd, 1H), 4.40 (s, 2H), 3.30-3.18 (m, 4H), 2.70 (s, 3H), which is initially charged in an autoclave with 1 g of Pd/C (10%) in 100 ml of methanol and subjected to a hydrogen pressure of 5 bar at 30° C. After 10 h, the catalyst is filtered off and the filtrate is concentrated under reduced pressure. This gives 2.20 g (78%) of 1-(3-aminobenzyl)-3-methylimidazolidin-2-one (IV-5) (logP (pH 2.3): 0.08). ¹H NMR (400 MHz, DMSO-d) δ=6.95 (dd, 1H), 6.46 (d, 1H), 6.44 (s, 1H), 6.36 (d, 1H), 4.85 (br.s, 2H), 4.09 (s, 2H), 3.23-3.09 (m, 4H), 2.67 (s, 3H).

### Preparation of Compounds of the Formulae (I), (Ia), (Ib) and (Ic)

### Method A According to Scheme 2

### Methyl 4-({5-chloro-4-[(1-methoxypropan-2-yl)amino]pyrimidin-2-yl}amino)benzene-carboxylate hydrochloride (compound 38)

At room temperature, 410 μl of a 4 M solution of HCl in dioxane are added to a solution of 250 mg (1.06 mmol) of 2,5-dichloro-N-(1-methoxypropan-2-yl)pyrimidine-4-amine and 352 mg (2.33 mmol) of methyl 4-aminobenzoate in 10 ml of acetonitrile, and the mixture is heated at 85° C. After 18 h, the hot reaction mixture is filtered and the filtrate is allowed to cool with stirring. The product precipitated from the filtrate is filtered off and dried. This gives 282 mg (71%) of the desired product (logP (pH 2.3): 2.31). ¹H NMR (400 MHz, DMSO-d) δ=9.60 (br. s, 1H), 8.02 (s, 1H), 7.86-7.81 (m, 4H), 6.76 (d, 1H), 4.45-4.42 (m, 1H), 3.81 (s, 3H), 3.54-3.50 (m, 2H), 3.41-3.38 (m, 2H), 1.22 (d, 3H).

### Method B According to Scheme 2

### 4-({5-Chloro-4-[(1-methoxypropan-2-yl}amino)-N,N-dimethylbenzene-sulphonamide (compound 16)

A mixture of 144 mg (0.72 mmol) of 2,5-dichloro-N-(1-methoxypropan-2-yl)pyrimidine-4-amine, 141 mg (0.69 mmol) of 4-amino-N,N-dimethylbenzenesulphonamide and 97 mg (0.51 mmol) of 4-toluenesulphonic acid in 10 ml of dioxane is stirred at 105° C. for 18 h. After cooling, the reaction is concentrated under reduced pressure and the residue is taken up in 50 ml of ethyl acetate. The organic phase is washed with 10 ml of saturated aq. NaHCO₃ and then with 10 ml of water, dried over MgSO₄ and freed from the solvent under reduced pressure. This gives 99 mg of the desired product (logP (pH 2.3): 2.25). ¹H NMR (400 MHz, DMSO-d) δ=10.53 (br. s, 1H), 8.19 (s, 1H), 7.91 (d, 2H), 7.75 (br. s, 1H), 7.69 (d, 2H), 4.48-4.46 (m, 1H), 3.55-3.52 (m, 1H), 3.40-3.37 (m, 1H), 3.26 (s, 3H), 2.59 (s, 6H), 1.22 (d, 3H).

### Preparation of Starting Materials of the Formula (VII) According to Scheme 3

### 2-Anilino-5-chloropyrimidin-4(3H)-one (VII-1)

A solution consisting of 3.27 ml of a 1 M aqueous NaOH solution and 1 ml of water is added to a solution of 500 mg (2.73 mmol) of 2,4,5-trichloropyrimidine in 10 ml of dioxane. After 4 d of stirring at room temperature, the reaction mixture is concentrated under reduced pressure. The residue is taken up in 50 ml of ethyl acetate and neutralized with 1 N HCl (aq). The organic phase is separated off and then washed with 10 ml of water, dried over MgSO₄ and freed from the solvent under reduced pressure. The crude product is, together with 424 mg (4.55 mmol) of aniline and 532 mg (3.09 mmol) of 4-toluenesulphonic acid, taken up in 10 ml of dioxane and heated at 105° C. with stirring. After 18 h, the reaction mixture is concentrated under reduced pressure and the residue is taken up in 50 ml of ethyl acetate. The organic phase is washed with 10 ml of saturated aq. NaHCO₃ and then with 10 ml of water, dried over MgSO₄ and freed from the solvent under reduced pressure. This gives 1000 mg of 2-anilino-5-chloropyrimidin-4(3H)-one (VII-1) which is directly reacted further, without any further purification (logP (pH 2.3): 1.56). ¹H NMR (400 MHz, DMSO-d) δ=7.71-7.68 (m, 2H), 7.51 (s, 1H), 7.49 (d, 1H), 7.16-7.10 (m, 3H), 6.75-6.74 (m, 1H).

### Preparation of Starting Materials of the Formula (VIII) According to Scheme 8

### 4,5-Dichloro-N-phenylpyrimidine-2-amine (VIII-1)

A solution of 400 mg of 2-anilino-5-chloropyrimidin-4(3H)-one in 2 ml of phosphoryl chloride is heated at 95° C. for 18 h. After cooling, the reaction mixture is concentrated under reduced pressure, added to water and extracted with dichloromethane (3×20 ml). The combined organic phases are dried over MgSO₄ and freed from the solvent under reduced pressure. This gives 450 mg of 4,5-dichloro-N-phenylpyrimidine-2-amine (VIII-1) (log P (pH 2.3): 3.52), ¹H NMR (400 MHz, DMSO-d) δ=9.31 (br, s, 1H), 8.33 (s, 1H), 7.60-7.57 (m, 2H), 7.40-7.36 (m, 2H), 7.2-7.16 (m, 1H).

The following compound can be prepared in an analogous manner:

5-bromo-2-chloro-N-phenylpyrimidine-2-amine (VIII-2) (log P (pH 2.3): 3.64).

### Preparation of Starting Materials of the Formula (VIII) According to Process 2d (Scheme 8)

### 4-Chloro-2-(phenylamino)pyrimidine-5-carbonitrile (VIII-3)

At 55° C., 1.76 g (25.5 mmol) of sodium nitrite are added a little at a time to a solution of 1.80 g (8.52 mmol) of 4-amino-2-(phenylamino)pyrimidine-5-carbonitrile (Lit: J Het. Chem. 1987, 24, 1305) in 22.5 ml of glacial acetic acid, and the mixture is stirred at this temperature for 18 h. The reaction mixture is cooled to 0° C., 150 ml of water are added and the mixture is extracted with ethyl acetate (5×50 ml). The combined organic phases are dried over MgSO₄ and freed from the solvent under reduced pressure. The product obtained is 4-hydroxy-2-(phenylamino)pyrimidine-5-carbonitrile (VII-2), which is, without any further work-up, reacted with 16 ml of phosphoryl chloride at 100° C. for 3 h. The reaction mixture is concentrated, and 100 ml of ice-water are then added to the residue and the mixture is extracted with dichloromethane (3×30 ml). The combined organic phases are dried over MgSO₄ and freed from the solvent under reduced pressure. The crude product is then purified by column chromatography on silica gel (cyclohexane/ethyl acetate). This gives 650 mg (16%) of 4-chloro-2-(phenylamino)pyrimidine-5-carbonitrile (VIII-3) (log P (pH 2.3): 2.66). ¹H NMR (400 MHz, DMSO-d) δ=10.65 (m, 1H), 8.87-8.83 (m, 1H), 7.70-7.64 (m, 2H), 7.41-7.34 (m, 2H), 7.15-7.115 (m, 1H).

### Preparation of Starting Materials of the Formula (VIII) According to Scheme 5

### 4-Chloro-N-[3-(propan-2-yl)phenyl]-5-(trifluoromethyl)pyrimidine-2-amine (VIII-4)

At 0° C., 2.76 ml of a 1M solution of zinc chloride in ether are added to a solution of 500 mg (2.30 mmol) of 2,4-dichloro-5-(trifluoromethyl)pyrimidine in 20 ml of t-butanol/dichloromethane (1:1), and the mixture is stirred for 1 h. 312 mg (2.30 mmol) of 3-isopropylaniline and 0.35 ml (2.54 mmol) of triethylamine, dissolved in 10 ml of butanol/dichloromethane (1:1), are then added, and the reaction mixture is allowed to warm to room temperature. After 16 h, the solvent is removed under reduced pressure, 40 ml of water are added to the residue and the mixture is extracted with ethyl acetate (3×50 ml). The combined organic phases are dried over MgSO₄ and freed from the solvent under reduced pressure. The crude product is then purified by column chromatography on silica gel (cyclohexane/ethyl acetate). This gives 140 mg (20%) of the desired product (log P (pH 2.3): 4.87). ¹H NMR (400 MHz, DMSO-d) δ=10.38 (m, 1H), 8.74 (d, 1H), 7.56 (m, 1H), 7.51-7.49 (m, 1H), 7.28-7.24 (m, 1H), 6.98 (m, 1H), 2.89-2.84 (mt, 1H), 1.23-1.21 (m, 6H).

### Preparation of Compounds of the Formulae (Ia), (Ib) and (Ic) According to Scheme 6

### 5-Chloro-N⁴-(2-methoxyethyl)-N⁴-methyl-N²-phenylpyrimidine-2,4-diamine (compound 222)

97 mg (1.09 mmol) of N-(2-methoxyethyl)-N-methylamine and 302 mg (2.17 mmol) of potassium carbonate are added to a solution of 250 mg (1.04 mmol) of 4,5-dichloro-N-phenylpyrimidine-2-amine in 5 ml of dimethylformamide, and the reaction mixture is heated at 90° C. for 16 h. After cooling, the reaction mixture is stirred into 20 ml of water and extracted with ethyl acetate (3×30 ml). The combined organic phases are dried over MgSO₄ and freed from the solvent under reduced pressure. This gives 259 mg (78%) of the desired product (logP (pH 2.3): 2.02). ¹H NMR (400 MHz, DMSO-d6) δ=9.02 (s, 1H), 7.96 (s, 1H), 7.64 (d, 2H), 7.23 (dd, 2H), 6.91 (dd, 1 H), 3.80 (t, 2H), 3.59 (t, 2H), 3.26 (s, 3H), 3.22 (s, 3H).

### 2-(Phenylamino)-4-(propylamino)pyrimidine-5-carbonitrile (compound 68)

50 mg (0.84 mmol) of n-propylamine and 73 mg (0.72 mmol) of triethylamine are added to a solution of 150 mg (0.65 mmol) of 4-chloro-2-(phenylamino)pyrimidine-5-carbonitrile in 5 ml of acetonitrile, and the reaction mixture is heated at 50° C. for 5 h. After cooling, the reaction mixture is stirred into 10 ml of water and extracted with dichloromethane (3×20 ml). The combined organic phases are dried over MgSO₄ and freed from the solvent under reduced pressure. This gives 170 mg (95%) of the desired product (logP (pH 2.3): 2.68). ¹H NMR (400 MHz, DMSO-d) δ=9.52 (m, 1H), 8.29-8.28 (m, 1H), 7.77-7.71 (m, 2H), 7.51 (br. s 1H), 7.29-7.25 (m, 2H), 7.01-9.97 (m, 1H), 3.40-3.35 (m, 2H), 2.66-2.53 (m, 2H), 0.93-0.88 (m, 3H).

### N⁴-Cyclopentyl-N²-[3-(propan-2-yl)phenyl]-5-(trifluoromethyl)pyrimidine-2,4-diamine (compound 257)

130 mg (1.52 mmol) of cyclopentylamine and 175 mg (1.27 mmol) of potassium carbonate are added to a solution of 400 mg (1.27 mmol) of 4-chloro-N-[3-(propan-2-yl)phenyl]-5-(trifluoromethyl)pyrimidine-2-amine in 20 ml of acetonitrile, and the reaction mixture is heated at 50° C. for 5 h. After cooling, the reaction mixture is stirred into 10 ml of water and extracted with dichloromethane (3×20 ml). The combined organic phases are dried over MgSO₄ and freed from the solvent under reduced pressure. This gives 364 mg (78%) of the desired product (logP (pH2.7): 5.08). ¹H NMR (400 MHz, DMSO-d) δ=9.32-9.29 (m, 1H), 8.15 (d, 1H), 4.70 (d, 1H), 7.19-7.15 (m, 1H), 6.87-6.85 (m, 1H), 6.18-6.16 (m, 1H), 4.58-4.53 (m, 1H), 2.88-2.82 (m, 1H), 2.03-1.96 (m, 2H), 1.74-1.69 (m, 2H), 1.58-1.53 (m, 4H), 1.22-1.20 (m, 6H).

### EXAMPLES

The compounds of the formulae (Ia), (Ib), (Ic) listed in Table I below are likewise obtained by the methods described above.

(Ia), (Ib) und (Ic)

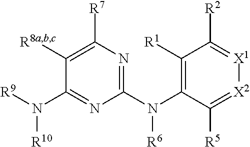

[und = and]

| Ex. | R5 | X² = CR⁴ | X¹ = CR³ | R² | R¹ | R⁶ | R⁷ | R⁸ | R⁹ | R¹⁰ | HX | logP |
|---|---|---|---|---|---|---|---|---|---|---|---|---|
| 1 | H | methoxy | methoxy | methoxy | H | H | H | Cl | H | 2-methoxyethyl | | 1.45[a]; |
| | | | | | | | | | | | | 1.45[b] |
| 2 | H | chloro | H | H | H | H | H | Cl | H | 2-methoxyethyl | | 2.13[a]; |
| | | | | | | | | | | | | 2.13[b] |
| 3 | H | H | H | H | H | H | H | Cl | H | 2-methoxyethyl | | 1.41[a] |
| 4 | H | methoxy | H | methoxy | H | H | H | Cl | H | 2-methoxyethyl | | 1.67[a] |
| 5 | H | acetyl | H | H | H | H | H | Cl | H | 2-methoxyethyl | | 1.49[a]; |
| | | | | | | | | | | | | 1.49[b] |
| 6 | H | H | dimethylsulphamoyl | H | H | H | H | Cl | H | 2-methoxyethyl | | 1.9[a]; |
| | | | | | | | | | | | | 1.9[b] |
| 7 | H | H | 4-(tert- | H | H | H | H | Cl | H | 2-methoxyethyl | | 2.05[a]; |
| | | | butoxycarbonyl)piper- | | | | | | | | | 2.05[b] |
| | | | azin-1-yl | | | | | | | | | |
| 8 | H | H | propan-2-yloxy | H | H | H | H | Cl | H | 1-methoxypropan-2- | | 1.69[a] |
| | | | | | | | | | | yl | | |
| 9 | H | H | propan-2-yl | H | H | H | H | Cl | H | 1-methoxypropan-2- | | 2.38[a] |
| | | | | | | | | | | yl | | |
| 10 | H | chloro | H | H | H | H | H | Cl | H | 1-methoxypropan-2- | | 2.58[a] |
| | | | | | | | | | | yl | | |
| 11 | H | H | propan-2-yl | H | H | H | H | Cl | H | 2-methoxyethyl | | 2.09[a] |
| 12 | H | H | propan-2-yloxy | H | H | H | H | Cl | H | 2-methoxyethyl | | 1.78[a] |
| 13 | H | H | diethylamino | H | H | H | H | Cl | H | 1-methoxypropan-2- | | 1.16[a] |
| | | | | | | | | | | yl | | |
| 14 | H | methoxy | H | methoxy | H | H | H | Cl | H | 1-methoxypropan-2- | | 1.94[a] |
| | | | | | | | | | | yl | | |
| 15 | H | acetyl | H | H | H | H | H | Cl | H | 1-methoxypropan-2- | | 1.72[a] |
| | | | | | | | | | | yl | | |
| 16 | H | H | dimethylsulphamoyl | H | H | H | H | Cl | H | 1-methoxypropan-2- | | 2.25[a]; |
| | | | | | | | | | | yl | | 2.26[b] |
| 17 | H | chloro | H | H | H | H | H | Cl | H | 1-hydroxybutan-2-yl | | 1.96[a] |
| 18 | H | H | H | H | H | H | H | Cl | H | 1-hydroxybutan-2-yl | | 1.41[a] |
| 19 | H | methoxy | H | methoxy | H | H | H | Cl | H | 1-hydroxybutan-2-yl | | 1.64[a] |
| 20 | H | methoxy | methoxy | methoxy | H | H | H | Cl | H | 1-hydroxybutan-2-yl | | 1.42[a] |
| 21 | H | acetyl | H | H | H | H | H | Cl | H | 1-hydroxybutan-2-yl | | 1.53[a] |
| 22 | H | H | dimethylsulphamoyl | H | H | H | H | Cl | H | 1-hydroxybutan-2-yl | | 1.83[a] |
| 23 | H | H | 4-(tert- | H | H | H | H | Cl | H | 1-hydroxybutan-2-yl | | 2.02[a] |
| | | | butoxycarbonyl)piper- | | | | | | | | | |
| | | | azin-1-yl | | | | | | | | | |
| 24 | H | methoxy | methoxy | methoxy | H | H | H | CF₃ | H | 1-methoxypropan-2- | | 2.46[b] |
| | | | | | | | | | | yl | | |
| 25 | H | H | H | H | H | H | H | CF₃ | H | 1-methoxypropan-2- | | 2.96[a]; |
| | | | | | | | | | | yl | | 3.52[c]; |
| | | | | | | | | | | | | 2.74[b] |
| 26 | H | H | dimethylsulphamoyl | H | H | H | H | Cl | H | CH₃ | | 1.61[a] |
| 27 | H | H | dimethylsulphamoyl | H | H | H | H | Cl | CH₃ | CH₃ | | 2.49[a] |
| 28 | H | H | methylsulphonyl | H | H | H | H | Cl | CH₃ | CH₃ | | 1.98[a] |
| 29 | H | morpholin-4- | H | H | H | H | H | Cl | H | 1-methoxypropan-2- | | 2.1[a]; |
| | | ylsulphonyl | | | | | | | | yl | | 2.04[b] |
| 30 | H | H | dimethylsulphamoyl | H | H | H | H | Cl | H | (2S)-1- | | 2.59[a] |
| | | | | | | | | | | (methylsulphanyl)pro- | | |
| | | | | | | | | | | pan-2-yl | | |
| 31 | H | dimethylsulphamoyl | H | H | H | H | H | Cl | H | 1-methoxypropan-2- | | 2.02[a]; |
| | | | | | | | | | | yl | | 2.18[b] |
| 32 | H | H | H | H | H | H | H | Cl | H | butyl | | 2.12[a] |
| 33 | H | H | H | H | H | H | H | Cl | CH₃ | CH₃ | | 1.78[a]; |
| | | | | | | | | | | | | 1.73[b] |
| 34 | H | H | H | H | H | H | H | Cl | H | tert-butyl | | 2.17[a] |
| 35 | H | H | H | H | H | H | H | Cl | H | CH₃ | | 1.27[a] |
| 36 | H | H | H | H | H | H | H | Cl | H | propan-2-yl | | 1.77[a] |
| 37 | H | H | H | H | H | H | H | Cl | H | (2R)-1- | | 2.04[a] |
| | | | | | | | | | | (methylsulphanyl)pro- | | |
| | | | | | | | | | | pan-2-yl | | |
| 38 | H | H | methoxycarbonyl | H | H | H | H | Cl | H | 1-methoxypropan-2- | | 2.31[a]; |
| | | | | | | | | | | yl | | 3.05[c] |
| 39 | H | H | methoxycarbonyl | H | H | H | H | Cl | CH₃ | CH₃ | | 2.47[a]; |
| | | | | | | | | | | | | 3.3[c] |
| 40 | H | H | H | H | H | H | H | Cl | CH₃ | butan-2-yl | | 2.85[a] |
| 41 | H | acetyl | H | H | H | H | H | Cl | CH₃ | butan-2-yl | HCl | 2.88[a] |
| 42 | H | acetyl | H | H | H | H | H | Cl | CH₃ | CH₃ | HCl | 1.84[a] |
| 43 | H | dimethylsulphamoyl | H | H | H | H | H | Cl | CH₃ | propan-2-yl | | 2.96[a] |
| 44 | H | H | H | H | H | H | H | Cyan | CH₃ | CH₃ | | 2.36[b] |
| 45 | H | acetyl | H | H | H | H | H | Cl | CH₃ | propan-2-yl | HCl | 2.5[a] |
| 46 | H | H | H | H | H | H | H | Cl | CH₃ | propan-2-yl | HCl | 2.45[a] |
| 47 | H | dimethylsulphamoyl | H | H | H | H | H | Cl | H | propan-2-yl | | 2.08[a] |
| 48 | H | (3-methyl-2- | H | H | H | H | H | Cl | H | propan-2-yl | | 2.56[c]; |
| | | oxoimidazolidin-1- | | | | | | | | | | 1.54[b] |
| | | yl)methyl | | | | | | | | | | |
| 49 | H | acetyl | H | H | H | H | H | Cl | H | tert-butyl | | 2.15[a] |
| 50 | H | acetyl | H | H | H | H | H | Cl | H | propan-2-yl | HCl | 1.79[a] |
| 51 | H | dimethylsulphamoyl | H | H | H | H | H | Cl | H | tert-butyl | | 2.73[a] |
| 52 | H | dimethylsulphamoyl | H | H | H | H | H | Cl | CH₃ | butan-2-yl | | 3.33[a] |
| 53 | H | H | H | H | H | H | H | Cl | H | 2-hydroxyethyl | HCl | 0.86[a] |
| 54 | H | methoxy | H | methoxy | H | H | H | Cl | H | 2-hydroxyethyl | HCl | 1.26[a] |
| 55 | H | methoxy | methoxy | methoxy | H | H | H | Cl | H | 2-hydroxyethyl | HCl | 1.09[a] |
| 56 | H | acetyl | H | H | H | H | H | Cl | H | 2-hydroxyethyl | HCl | 1.06[a] |
| 57 | H | H | dimethylsulphamoyl | H | H | H | H | Cl | H | 2-hydroxyethyl | HCl | 1.4[a] |
| 58 | H | H | propan-2-yl | H | H | H | H | Cl | H | 2-hydroxyethyl | HCl | 1.6[a] |
| 59 | H | H | propan-2-yloxy | H | H | H | H | Cl | H | 2-hydroxyethyl | HCl | 1.48[a] |
| 60 | H | H | H | H | H | H | H | Cl | ethyl | ethyl | | 2.29[a] |
| 61 | H | H | H | H | H | H | H | Cl | H | 2-methoxyethyl | HCl | 1.41[a]; |
| | | | | | | | | | | | | 1.41[b] |
| 62 | H | methoxy | H | methoxy | H | H | H | Cl | H | 2-methoxyethyl | HCl | 1.67[a]; |
| | | | | | | | | | | | | 1.67[b] |
| 63 | H | H | morpholin-4- | H | H | H | H | Cl | H | 1-methoxypropan-2- | | 2.18[a] |
| | | | ylsulphonyl | | | | | | | yl | | |
| 64 | H | H | morpholin-4- | H | H | H | H | Cl | H | tert-butyl | | 2.84[a] |
| | | | ylsulphonyl | | | | | | | | | |
| 65 | H | H | H | H | H | H | H | Cl | H | butan-2-yl | HCl | 2.04[a] |
| 66 | H | H | H | H | H | H | H | Cl | H | prop-2-en-1-yl | HCl | 1.68[a] |
| 67 | H | H | H | H | H | H | H | cyano | H | 2-(methylsulphanyl)- | | 2.6[b] |
| | | | | | | | | | | ethyl | | |
| 68 | H | H | H | H | H | H | H | cyano | H | propyl | | 2.68[b] |
| 69 | H | H | fluoro | H | H | H | H | Cl | H | 1-methoxypropan-2- | | 1.8[b] |
| | | | | | | | | | | yl | | |
| 70 | H | H | fluoro | H | H | H | H | Br | H | 1-methoxypropan-2- | | 1.95[b] |
| | | | | | | | | | | yl | | |
| 71 | H | chloro | H | H | H | H | H | Br | H | 1-methoxypropan-2- | | 2.82[b] |
| | | | | | | | | | | yl | | |
| 72 | H | dimethylsulphamoyl | H | H | H | H | H | Br | H | 1-methoxypropan-2- | | 2.37[b] |
| | | | | | | | | | | yl | | |
| 73 | fluoro | H | fluoro | H | H | H | H | Br | H | 1-methoxypropan-2- | | 2.16[b] |
| | | | | | | | | | | yl | | |
| 74 | H | methoxy | methoxy | H | H | H | H | Br | H | 1-methoxypropan-2- | | 1.55[b] |
| | | | | | | | | | | yl | | |
| 75 | H | —OCF₂CF₂O— | | H | H | H | H | Br | H | 1-methoxypropan-2- | | 4.08[b] |
| | | | | | | | | | | yl | | |
| 76 | H | methylsulphonyl | chloro | H | H | H | H | Br | H | 1-methoxypropan-2- | | 2.23[b] |
| | | | | | | | | | | yl | | |
| 77 | H | methylsulphanyl | H | H | H | H | H | Br | H | 1-methoxypropan-2- | | 2.3[b] |
| | | | | | | | | | | yl | | |
| 78 | H | H | propanoyl | H | H | H | H | Br | H | 1-methoxypropan-2- | | 2.57[b] |
| | | | | | | | | | | yl | | |
| 79 | H | —CH₂CH₂N(COCH₃)— | | H | H | H | H | Br | H | 1-methoxypropan-2- | | 1.55[b] |
| | | | | | | | | | | yl | | |
| 80 | H | acetylamino | H | H | H | H | H | Br | H | 1-methoxypropan-2- | | 1.11[b] |
| | | | | | | | | | | yl | | |
| 81 | fluoro | H | fluoro | H | H | H | H | Cl | H | 1-methoxypropan-2- | | 2.02[b] |
| | | | | | | | | | | yl | | |
| 82 | H | H | CF₃ | H | H | H | H | Cl | H | 1-methoxypropan-2- | | 3.22[b] |
| | | | | | | | | | | yl | | |
| 83 | H | cyano | H | H | H | H | H | Cl | H | 1-methoxypropan-2- | | 2.11[b] |
| | | | | | | | | | | yl | | |
| 84 | H | methoxy | methoxy | H | H | H | H | Cl | H | 1-methoxypropan-2- | | 1.46[b] |
| | | | | | | | | | | yl | | |
| 85 | H | methoxy | H | H | H | H | H | Cl | H | 1-methoxypropan-2- | | 1.78[b] |
| | | | | | | | | | | yl | | |
| 86 | H | H | methoxy | H | H | H | H | Cl | H | 1-methoxypropan-2- | | 1.56[b] |
| | | | | | | | | | | yl | | |
| 87 | H | trifluoromethoxy | H | H | H | H | H | Cl | H | 1-methoxypropan-2- | | 3.14[b] |
| | | | | | | | | | | yl | | |
| 88 | H | H | (trifluoromethyl)- | H | H | H | H | Cl | H | 1-methoxypropan-2- | | 3.81[b] |
| | | | sulphanyl | | | | | | | yl | | |
| 89 | H | chloro | methylsulphanyl | H | H | H | H | Cl | H | 1-methoxypropan-2- | | 2.77[b] |
| | | | | | | | | | | yl | | |
| 90 | H | H | 1,3-thiazol-4-yl | H | H | H | H | Cl | H | 1-methoxypropan-2- | | 1.87[b] |
| | | | | | | | | | | yl | | |
| 91 | H | H | 2-methoxy-2-oxoethyl | H | H | H | H | Cl | H | 1-methoxypropan-2- | | 1.71[b] |
| | | | | | | | | | | yl | | |
| 92 | H | —OCF₂CF₂O— | | H | H | H | H | Cl | H | 1-methoxypropan-2- | | 3.85[b] |
| | | | | | | | | | | yl | | |
| 93 | H | 2-methoxy-2-oxoethyl | H | H | H | H | H | Cl | H | 1-methoxypropan-2- | | 1.75[b] |
| | | | | | | | | | | yl | | |
| 94 | H | chloro | H | chloro | H | H | H | Cl | H | 1-methoxypropan-2- | | 4.08[b] |
| | | | | | | | | | | yl | | |
| 95 | H | chloro | methoxycarbonyl | H | H | H | H | Cl | H | 1-methoxypropan-2- | | 2.95[b] |
| | | | | | | | | | | yl | | |
| 96 | H | ethoxy | H | H | H | H | H | Cl | H | 1-methoxypropan-2- | | 2.04[b] |
| | | | | | | | | | | yl | | |
| 97 | H | methylenbis(oxy) | | H | H | H | H | Cl | H | 1-methoxypropan-2- | | 1.56[b] |
| | | | | | | | | | | yl | | |
| 98 | H | chloro | trifluoromethoxy | H | H | H | H | Cl | H | 1-methoxypropan-2- | | 3.99[b] |
| | | | | | | | | | | yl | | |
| 99 | H | H | acetyl(methyl)amino | H | H | H | H | Cl | H | 1-methoxypropan-2- | | 1.48[b] |
| | | | | | | | | | | yl | | |
| 100 | H | methylsulphonyl | chloro | H | H | H | H | Cl | H | 1-methoxypropan-2- | | 2.09[b] |
| | | | | | | | | | | yl | | |
| 101 | H | methylsulphanyl | H | H | H | H | H | Cl | H | 1-methoxypropan-2- | | 2.13[b] |
| | | | | | | | | | | yl | | |
| 102 | H | H | cyanoacetyl | H | H | H | H | Cl | H | 1-methoxypropan-2- | | 2.09[b] |
| | | | | | | | | | | yl | | |
| 103 | H | —CH₂CH₂CO— | | H | H | H | H | Cl | H | 1-methoxypropan-2- | | 1.97[b] |
| | | | | | | | | | | yl | | |
| 104 | H | H | propanoyl | H | H | H | H | Cl | H | 1-methoxypropan-2- | | 2.36[b] |
| | | | | | | | | | | yl | | |
| 105 | H | —N═C(CH₃)S— | | H | H | H | H | Cl | H | 1-methoxypropan-2- | | 1.75[b] |
| | | | | | | | | | | yl | | |
| 106 | H | —SCOO— | | H | H | H | H | Cl | H | 1-methoxypropan-2- | | 2.11[b] |
| | | | | | | | | | | yl | | |
| 107 | H | —CH₂CH₂N(COCH₃)— | | H | H | H | H | Cl | H | 1-methoxypropan-2- | | 1.48[b] |
| | | | | | | | | | | yl | | |
| 108 | H | cyano | H | H | H | H | H | Br | H | 1-methoxypropan-2- | | 2.26[b] |
| | | | | | | | | | | yl | | |
| 109 | H | methoxy | H | H | H | H | H | Br | H | 1-methoxypropan-2- | | 1.9[b] |
| | | | | | | | | | | yl | | |
| 110 | H | H | methoxy | H | H | H | H | Br | H | 1-methoxypropan-2- | | 1.63[b] |
| | | | | | | | | | | yl | | |
| 111 | H | chloro | methylsulphanyl | H | H | H | H | Br | H | 1-methoxypropan-2- | | 2.99[b] |
| | | | | | | | | | | yl | | |
| 112 | H | H | 1,3-thiazol-4-yl | H | H | H | H | Br | H | 1-methoxypropan-2- | | 1.99[b] |
| | | | | | | | | | | yl | | |
| 113 | H | 2-methoxy-2-oxoethyl | H | H | H | H | H | Br | H | 1-methoxypropan-2- | | 1.87[b] |
| | | | | | | | | | | yl | | |
| 114 | H | chloro | H | chloro | H | H | H | Br | H | 1-methoxypropan-2- | | 4.32[b] |
| | | | | | | | | | | yl | | |
| 115 | H | chloro | methoxycarbonyl | H | H | H | H | Br | H | 1-methoxypropan-2- | | 3.14[b] |
| | | | | | | | | | | yl | | |
| 116 | H | ethoxy | H | H | H | H | H | Br | H | 1-methoxypropan-2- | | 2.18[b] |
| | | | | | | | | | | yl | | |
| 117 | H | methylenebis(oxy) | | H | H | H | H | Br | H | 1-methoxypropan-2- | | 1.64[b] |
| | | | | | | | | | | yl | | |
| 118 | H | chloro | trifluoromethoxy | H | H | H | H | Br | H | 1-methoxypropan-2- | | 4.23[b] |
| | | | | | | | | | | yl | | |
| 119 | H | H | acetyl(methyl)amino | H | H | H | H | Br | H | 1-methoxypropan-2- | | 1.56[b] |
| | | | | | | | | | | yl | | |
| 120 | H | morpholin-4- | H | H | H | H | H | Br | H | 1-methoxypropan-2- | | 2.16[b] |
| | | ylsulphonyl | | | | | | | | yl | | |
| 121 | H | H | cyanoacetyl | H | H | H | H | Br | H | 1-methoxypropan-2- | | 2.23[b] |
| | | | | | | | | | | yl | | |
| 122 | H | —N═C(CH₃)S— | | H | H | H | H | Br | H | 1-methoxypropan-2- | | 1.87[b] |
| | | | | | | | | | | yl | | |
| 123 | H | —SCOO— | | H | H | H | H | Br | H | 1-methoxypropan-2- | | 2.26[b] |
| | | | | | | | | | | yl | | |
| 124 | H | methylcarbamoyl | H | H | H | H | H | Br | H | 1-methoxypropan-2- | | 1.4[b] |
| | | | | | | | | | | yl | | |
| 125 | H | methoxy | acetylamino | H | H | H | H | Br | H | 1-methoxypropan-2- | | 1.46[b] |
| | | | | | | | | | | yl | | |
| 126 | H | H | phenylcarbamoyl | H | H | H | H | Br | H | 1-methoxypropan-2- | | 1.92[b] |
| | | | | | | | | | | yl | | |
| 127 | H | H | methylcarbamoyl | H | H | H | H | Br | H | 1-methoxypropan-2- | | 1.46[b] |
| | | | | | | | | | | yl | | |
| 128 | H | H | prop-2-en-1- | H | H | H | H | Br | H | 1-methoxypropan-2- | | 1.75[b] |
| | | | ylcarboamoyl | | | | | | | yl | | |
| 129 | H | (ethoxycarbonyl)amino | H | H | H | H | H | Br | H | 1-methoxypropan-2- | | 1.87[b] |
| | | | | | | | | | | yl | | |
| 130 | H | (methylcarbamoyl)oxy | H | H | H | H | H | Br | H | 1-methoxypropan-2- | | 1.56[b] |
| | | | | | | | | | | yl | | |
| 131 | H | H | formylamino | H | H | H | H | Br | H | 1-methoxypropan-2- | | 1.3[b] |
| | | | | | | | | | | yl | | |
| 132 | H | formylamino | H | H | H | H | H | Br | H | 1-methoxypropan-2- | | 1.43[b] |
| | | | | | | | | | | yl | | |
| 133 | H | —CH₂CH₂CONH— | | H | H | H | H | Br | H | 1-methoxypropan-2- | | 1.38[b] |
| | | | | | | | | | | yl | | |
| 134 | H | —OCONH— | | H | H | H | H | Br | H | 1-methoxypropan-2- | | 1.29[b] |
| | | | | | | | | | | yl | | |
| 135 | H | H | methylcarbamoyl | H | H | H | H | Cl | H | 1-methoxypropan-2- | | 1.38[b] |
| | | | | | | | | | | yl | | |
| 136 | H | H | prop-2-en-1- | H | H | H | H | Cl | H | 1-methoxypropan-2- | | 1.64[b] |
| | | | ylcarbamoyl | | | | | | | yl | | |
| 137 | H | H | tert-butylcarbamoyl | H | H | H | H | Cl | H | 1-methoxypropan-2- | | 2.06[b] |
| | | | | | | | | | | yl | | |
| 138 | H | H | formylamino | H | H | H | H | Cl | H | 1-methoxypropan-2- | | 1.28[b] |
| | | | | | | | | | | yl | | |
| 139 | H | —CH₂CH₂CONH— | | H | H | H | H | Cl | H | 1-methoxypropan-2- | | 1.3[b] |
| | | | | | | | | | | yl | | |
| 140 | H | methoxy | acetylamino | H | H | H | H | Cl | H | 1-methoxypropan-2- | | 1.38[b] |
| | | | | | | | | | | yl | | |
| 141 | H | H | phenylcarbamoyl | H | H | H | H | Cl | H | 1-methoxypropan-2- | | 1.8[b] |
| | | | | | | | | | | yl | | |
| 142 | H | methylcarbamoyl | H | H | H | H | H | Cl | H | 1-methoxypropan-2- | | 1.32[b] |
| | | | | | | | | | | yl | | |
| 143 | H | (ethoxycarbonyl)amino | H | H | H | H | H | Cl | H | 1-methoxypropan-2- | | 1.78[b] |
| | | | | | | | | | | yl | | |
| 144 | H | (methylcarbamoyl)oxy | H | H | H | H | H | Cl | H | 1-methoxypropan-2- | | 1.48[b] |
| | | | | | | | | | | yl | | |
| 145 | H | H | methylsulphonyl | H | H | H | H | Cl | H | 1-methoxypropan-2- | | 1.85[b] |
| | | | | | | | | | | yl | | |
| 146 | H | H | (2,2- | H | H | H | H | Cl | H | 1-methoxypropan-2- | | 1.78[b] |
| | | | dimethylpropanoyl)- | | | | | | | yl | | |
| | | | amino | | | | | | | | | |
| 147 | H | (2,2-dimethyl- | H | H | H | H | H | Cl | H | 1-methoxypropan-2- | | 1.9[b] |
| | | propanoyl)amino | | | | | | | | yl | | |
| 148 | H | H | dimethylcarbamoyl | H | H | H | H | Cl | H | 1-methoxypropan-2- | | 1.51[b] |
| | | | | | | | | | | yl | | |
| 149 | H | H | methylsulphanyl | H | H | H | H | Cl | H | 1-methoxypropan-2- | | 2.06[b] |
| | | | | | | | | | | yl | | |
| 150 | H | piperidin-1-ylsulphonyl | H | H | H | H | H | Cl | H | 1-methoxypropan-2- | | 2.77[b] |
| | | | | | | | | | | yl | | |
| 151 | H | H | 3,3-dimethyl-2-oxobutyl | H | H | H | H | Cl | H | 1-methoxypropan-2- | | 2.26[b] |
| | | | | | | | | | | yl | | |
| 152 | H | H | acetyl | H | H | H | H | Cl | H | 1-methoxypropan-2- | | 2.02[b] |
| | | | | | | | | | | yl | | |
| 153 | H | (methoxycarbonyl)- | H | H | H | H | H | Cl | H | 1-methoxypropan-2- | | 1.59[b] |
| | | amino | | | | | | | | yl | | |
| 154 | H | methylsulphanyl | CH₃ | H | H | H | H | Cl | H | 1-methoxypropan-2- | | 2.33[b] |
| | | | | | | | | | | yl | | |
| 155 | H | dimethylcarbamoyl | H | H | H | H | H | Cl | H | 1-methoxypropan-2- | | 1.47[b] |
| | | | | | | | | | | yl | | |
| 156 | H | (2-methylpropanoyl)- | H | H | H | H | H | Cl | H | 1-methoxypropan-2- | | 1.68[b] |
| | | amino | | | | | | | | yl | | |
| 157 | H | tert-butylcarbamoyl | H | H | H | H | H | Cl | H | 1-methoxypropan-2- | | 1.99[b] |
| | | | | | | | | | | yl | | |
| 158 | H | H | 1,1,2,2-tetrafluoroethoxy | H | H | H | H | Cl | H | 1-methoxypropan-2- | | 2.61[b] |
| | | | | | | | | | | yl | | |
| 159 | H | acetyl | hydroxy | H | H | H | H | Cl | H | 1-methoxypropan-2- | | 1.68[b] |
| | | | | | | | | | | yl | | |
| 160 | H | fluoro | hydroxy | H | H | H | H | Cl | H | 1-methoxypropan-2- | | 1.34[b] |
| | | | | | | | | | | yl | | |
| 161 | H | H | acetyl(cyclohexyl)amino | H | H | H | H | Cl | H | 1-methoxypropan-2- | | 2.33[b] |
| | | | | | | | | | | yl | | |
| 162 | H | [(1-methylcyclo- | H | H | H | H | H | Cl | H | 1-methoxypropan-2- | | 1.78[b] |
| | | propyl)carbonyl]amino | | | | | | | | yl | | |
| 163 | H | H | (2,2- | H | H | H | H | Cl | H | 1-methoxypropan-2- | | 1.87[b] |
| | | | dimethylpropanoyl)- | | | | | | | yl | | |
| | | | (methyl)amino | | | | | | | | | |
| 164 | H | H | cyclopropyl(trifluor- | H | H | H | H | Cl | H | 1-methoxypropan-2- | | 2.55[b] |
| | | | acetyl)amino | | | | | | | yl | | |
| 165 | H | (phenoxycarbonyl)- | H | H | H | H | H | Cl | H | 1-methoxypropan-2- | | 2.23[b] |
| | | amino | | | | | | | | yl | | |
| 166 | H | (3-fluoro-2,2-dimethyl- | H | H | H | H | H | Cl | H | 1-methoxypropan-2- | | 1.83[b] |
| | | propanoyl)amino | | | | | | | | yl | | |
| 167 | H | acetylamino | CH₃ | H | H | H | H | Cl | H | 1-methoxypropan-2- | | 1.37[b] |
| | | | | | | | | | | yl | | |
| 168 | H | (furan-2-ylcarbonyl)- | H | H | H | H | H | Cl | H | 1-methoxypropan-2- | | 1.68[b] |
| | | amino | | | | | | | | yl | | |
| 169 | H | H | (2- | H | H | H | H | Cl | H | 1-methoxypropan-2- | | 1.59[b] |
| | | | methylpropanoyl)amino | | | | | | | yl | | |
| 170 | H | —N(CH₃)COCH₂O— | | H | H | H | H | Cl | H | 1-methoxypropan-2- | | 1.56[b] |
| | | | | | | | | | | yl | | |
| 171 | H | CH₃ | hydroxy | H | H | H | H | Cl | H | 1-methoxypropan-2- | | 1.39[b] |
| | | | | | | | | | | yl | | |
| 172 | H | H | (difluoromethyl)sul- | H | H | H | H | Cl | H | 1-methoxypropan-2- | | 2.8[b] |
| | | | phanyl | | | | | | | yl | | |
| 173 | H | methylsulphanyl | H | H | H | H | H | Cl | H | propan-2-yl | | 2.36[a]; |
| | | | | | | | | | | | | 3.89[c] |
| 174 | H | H | H | H | H | H | H | Cl | H | 2-methyl-1- | | 2.68[a]; |
| | | | | | | | | | | (methylsulphanyl)- | | 4.18[c] |
| | | | | | | | | | | propan-2-yl | | |
| 175 | H | methylsulphanyl | H | H | H | H | H | Cl | H | 2-methyl-1- | | 3.48[a]; |
| | | | | | | | | | | (methylsulphanyl)- | | 4.59[c] |
| | | | | | | | | | | propan-2-yl | | |
| 176 | H | H | H | H | H | H | H | Cl | H | 1-methoxy-3- | | 2.33[b] |
| | | | | | | | | | | methylbutan-2-yl | | |
| 177 | H | H | H | H | H | H | H | Cl | H | 2-(trifluoromethoxy)- | | 2.26[b] |
| | | | | | | | | | | ethyl | | |
| 178 | H | H | H | H | H | H | H | Br | CH₃ | CH₃ | | 1.86[b] |
| 179 | H | acetyl | H | H | H | H | H | Cl | CH₃ | butan-2-yl | | 2.88[a] |
| 180 | H | cyano | H | H | H | H | H | Cl | H | propan-2-yl | | 2.21[b] |
| 181 | H | methoxy | acetylamino | H | H | H | H | Cl | H | propan-2-yl | | 1.44[b] |
| 182 | H | methoxy | H | H | H | H | H | Cl | H | propan-2-yl | | 1.86[b] |
| 183 | H | H | methoxy | H | H | H | H | Cl | H | propan-2-yl | | 1.63[b] |
| 184 | H | H | cyano | H | H | H | H | Cl | H | propan-2-yl | | 2.46[b] |
| 185 | H | trifluoromethoxy | H | H | H | H | H | Cl | H | propan-2-yl | | 3.26[b] |
| 186 | H | H | methylsulphonyl | H | H | H | H | Cl | H | propan-2-yl | | 1.86[b] |
| 187 | H | chloro | methylsulphanyl | H | H | H | H | Cl | H | propan-2-yl | | 2.91[b] |
| 188 | H | H | (2,2-dimethyl- | H | H | H | H | Cl | H | propan-2-yl | | 1.81[b] |
| | | | propanoyl)amino | | | | | | | | | |
| 189 | H | H | acetylamino | H | H | H | H | Cl | H | propan-2-yl | | 1.32[b] |
| 190 | H | chloro | acetylamino | H | H | H | H | Cl | H | propan-2-yl | | 1.61[b] |
| 191 | H | H | acetyl(ethyl)amino | H | H | H | H | Cl | H | propan-2-yl | | 1.66[b] |
| 192 | H | H | acetyl(methyl)amino | H | H | H | H | Cl | H | propan-2-yl | | 1.51[b] |
| 193 | H | H | (trifluoracetyl)amino | H | H | H | H | Cl | H | propan-2-yl | | 1.93[b] |
| 194 | H | H | [(1-methylcyclo- | H | H | H | H | Cl | H | propan-2-yl | | 1.71[b] |
| | | | propyl)carbonyl]amino | | | | | | | | | |
| 195 | H | acetylamino | H | H | H | H | H | Cl | H | propan-2-yl | | 1.39[b] |
| 196 | H | (2-methylacryloyl)- | H | H | H | H | H | Cl | H | propan-2-yl | | 1.66[b] |
| | | amino | | | | | | | | | | |
| 197 | H | (2,2-dimethyl- | H | H | H | H | H | Cl | H | propan-2-yl | | 1.91[b] |
| | | propanoyl)amino | | | | | | | | | | |
| 198 | H | piperidin-1-ylsulphonyl | H | H | H | H | H | Cl | H | propan-2-yl | | 2.08[b] |
| 199 | H | H | 3,3-dimethyl-2-oxobutyl | H | H | H | H | Cl | H | propan-2-yl | | 2.3[b] |
| 200 | H | H | acetyl | H | H | H | H | Cl | H | propan-2-yl | | 2.06[b] |
| 201 | H | H | (methoxycarbonyl)amino | H | H | H | H | Cl | H | propan-2-yl | | 1.53[b] |
| 202 | H | H | (ethoxycarbonyl)amino | H | H | H | H | Cl | H | propan-2-yl | | 1.66[b] |
| 203 | H | (methylcarbamoyl)oxy | H | H | H | H | H | Cl | H | propan-2-yl | | 1.53[b] |
| 204 | H | (methoxycarbonyl)amino | H | H | H | H | H | Cl | H | propan-2-yl | | 1.59[b] |
| 205 | H | —OCONH— | | H | H | H | H | Cl | H | propan-2-yl | | 1.44[b] |
| 206 | H | —N(COCH₃)CH₂CH₂— | | H | H | H | H | Cl | H | propan-2-yl | | 1.61[b] |
| 207 | H | [(1-methylcyclo- | H | H | H | H | H | Cl | H | propan-2-yl | | 1.78[b] |
| | | propyl)carbonyl]amino | | | | | | | | | | |
| 208 | H | acetylamino | CH₃ | H | H | H | H | Cl | H | propan-2-yl | | 1.42[b] |
| 209 | H | H | (2- | H | H | H | H | Cl | H | propan-2-yl | | 1.61[b] |
| | | | methylpropanoyl)amino | | | | | | | | | |
| 210 | H | —NHCOCH₂CH₂CH₂— | | H | H | H | H | Cl | H | propan-2-yl | | 1.56[b] |
| 211 | H | 1,1,2,2-tetrafluorethoxy | H | H | H | H | H | Cl | H | propan-2-yl | | 2.88[b] |
| 212 | H | H | (difluoromethyl)sul- | H | H | H | H | Cl | H | propan-2-yl | | 2.88[b] |
| | | | phanyl | | | | | | | | | |
| 213 | H | H | hydroxy | H | H | H | H | Cl | H | propan-2-yl | | 1.24[b] |
| 214 | H | hydroxy | H | H | H | H | H | Cl | H | propan-2-yl | | 1.39[b] |
| 215 | H | —NHCH═CH— | | H | H | H | H | Cl | H | propan-2-yl | | 1.63[b] |
| 216 | H | (ethoxycarbonyl)amino | H | H | H | H | H | Cl | H | propan-2-yl | | 1.81[b] |
| 217 | H | —COOCH₂— | | H | H | H | H | Cl | H | 1-methoxypropan-2- | | 1.61[b] |
| | | | | | | | | | | yl | | |
| 218 | H | —COOCH₂— | | H | H | H | H | Br | H | 1-methoxypropan-2- | | 1.73[b] |
| | | | | | | | | | | yl | | |
| 219 | H | methylsulphanyl | H | H | H | H | H | Cl | CH₃ | propan-2-yl | | 3.45[a]; |
| | | | | | | | | | | | | 4.64[c] |
| 220 | H | H | H | H | H | H | H | Cl | H | cyanomethyl | | 1.62[a]; |
| | | | | | | | | | | | | 2.2[c] |
| 221 | H | methylsulphanyl | H | H | H | H | H | Cl | H | cyanomethyl | | 2.27[a] |
| 222 | H | H | H | H | H | H | H | Cl | CH₃ | 2-methoxyethyl | | 2.02[a] |
| 223 | H | H | H | H | H | H | H | Cl | H | 1-hydroxypropan-2-yl | | 1.04[a] |
| 224 | H | H | H | H | H | H | H | Br | H | 1-methoxy-3- | | 2.55[b] |
| | | | | | | | | | | methylbutan-2-yl | | |
| 225 | H | CH₃ | H | H | H | H | H | Cl | H | 1-methoxypropan-2- | | 1.98[b] |
| | | | | | | | | | | yl | | |
| 226 | H | H | H | H | H | H | H | Br | H | 1-methoxypropan-2- | | 2.52[a] |
| | | | | | | | | | | yl | | |
| 227 | H | H | 3-methyl-2,5- | H | H | H | H | Cl | H | 1-methoxypropan-2- | | 1.41[b] |
| | | | dioxoimidazolidin-1-yl | | | | | | | yl | | |
| 228 | H | H | H | H | H | H | H | Br | H | hexan-3-yl | | 2.44[a] |
| 229 | H | CH₃ | H | H | H | H | H | Cl | H | propan-2-yl | | 3.84[c]; |
| | | | | | | | | | | | | 2[b] |
| 230 | H | ethyl | H | H | H | H | H | Cl | H | propan-2-yl | | 4.25[c]; |
| | | | | | | | | | | | | 2.33[b] |
| 231 | H | H | H | H | H | H | H | Br | CH₃ | 1-methoxy-3- | | 2.47[a] |
| | | | | | | | | | | methylbutan-2-yl | | |
| 232 | H | H | H | H | H | H | H | Br | CH₃ | 1-methoxybutan-2-yl | | 2.23[a] |
| 233 | H | H | H | H | H | H | H | Br | CH₃ | 4-methylpentan-2-yl | | 3.39[d] |
| 234 | H | H | dimethylcarbamoyl | H | H | H | H | Cl | H | propan-2-yl | | 2.27[c]; |
| | | | | | | | | | | | | 1.44[b] |
| 235 | H | cyanomethyl | H | H | H | H | H | Cl | H | CH₃ | | 1.1[b] |
| 236 | H | cyanomethyl | H | H | H | H | H | Cl | H | propan-2-yl | | 1.69[c] |
| 237 | H | H | H | H | H | H | H | Br | CH₃ | 2-ethoxyethyl | | 3.95[c]; |
| | | | | | | | | | | | | 2.55[b] |
| 238 | H | methylsulphanyl | H | H | H | H | H | Cl | H | ethyl | | 3.46[c]; |
| | | | | | | | | | | | | 1.92[b] |
| 239 | H | methylsulphanyl | H | H | H | H | H | Cl | CH₃ | ethyl | | 4.32[c]; |
| | | | | | | | | | | | | 2.99[b] |
| 240 | H | H | H | H | H | H | H | Cl | H | ethyl | | 3.03[c]; |
| | | | | | | | | | | | | 1.38[b] |
| 241 | H | H | H | H | H | H | H | Cl | CH₃ | ethyl | | 3.85[c]; |
| | | | | | | | | | | | | 2.11[b] |
| 242 | H | [methyl(propan-2- | H | H | H | H | H | Cl | H | propan-2-yl | | 3.44[c]; |
| | | yl)sulphamoyl]methyl | | | | | | | | | | 2.18[b] |
| 243 | H | [ethyl(methyl)sulph- | H | H | H | H | H | Cl | H | propan-2-yl | | 3.18[c]; |
| | | amoyl]methyl | | | | | | | | | | 1.96[b] |
| 244 | H | formyl | H | H | H | H | H | Cl | H | 1-methoxypropan-2- | | 2.75[c]; |
| | | | | | | | | | | yl | | 1.8[b] |
| 245 | H | H | dimethylcarbamoyl | H | H | H | H | Br | H | propan-2-yl | | 1.54[b] |
| 246 | H | cyanomethyl | H | H | H | H | H | Br | H | propan-2-yl | | 3.13[c]; |
| | | | | | | | | | | | | 1.84[b] |
| 247 | H | H | H | H | H | H | H | Br | H | propan-2-yl | | 3.7[c]; |
| | | | | | | | | | | | | 1.89[b] |
| 248 | H | [(1-phenylethyl)sulph- | H | H | H | H | H | Cl | H | propan-2-yl | | 3.57[c]; |
| | | amoyl]methyl | | | | | | | | | | 2.32[b] |
| 249 | H | acetyl | H | H | H | H | H | Cl | H | cyclohexyl | HCl | 2.53[b] |
| 250 | H | (dimethylsulphamoyl)- | H | H | H | H | H | Cl | H | propan-2-yl | | 2.85[c]; |
| | | methyl | | | | | | | | | | 1.69[b] |
| 251 | H | (benzylsulphamoyl)- | H | H | H | H | H | Cl | H | propan-2-yl | | 3.35[c]; |
| | | methyl | | | | | | | | | | 2.18[b] |
| 252 | H | formyl | H | H | H | H | H | Cl | H | propan-2-yl | | 3.06[c]; |
| | | | | | | | | | | | | 1.86[b] |
| 253 | H | H | H | H | H | H | H | Br | H | cyclopentyl | | 2.47[b] |
| 254 | H | H | H | H | H | H | H | Cl | H | cyclopentyl | | 2.28[b] |
| 255 | H | 2-oxopropyl | H | H | H | H | H | Cl | H | cyclopentyl | | 2.01[b] |
| 256 | H | methylsulphanyl | H | H | H | H | H | Cl | H | cyclopentyl | | 2.93[b] |
| 257 | H | propan-2-yl | H | H | H | H | H | CF₃ | H | cyclopentyl | | 5.08[b] |
| 258 | H | chloro | H | H | H | H | H | Cl | H | cyclohexyl | | 3.81[b] |
| 259 | H | H | H | H | H | H | H | Cl | H | cyclohexyl | | 2.53[b] |
| 260 | H | methoxy | H | methoxy | H | H | H | Cl | H | cyclohexyl | | 2.77[b] |
| 261 | H | methoxy | methoxy | methoxy | H | H | H | Cl | H | cyclohexyl | | 2.26[b] |
| 262 | H | acetyl | H | H | H | H | H | Cl | H | cyclohexyl | | 2.53[a] |
| 263 | H | H | dimethylsulphamoyl | H | H | H | H | Cl | H | cyclohexyl | | 3.18[b] |
| 264 | H | H | 4-(tert- | H | H | H | H | Cl | H | cyclohexyl | | 2.95[b] |
| | | | butoxycarbonyl)piperazin- | | | | | | | | | |
| | | | 1-yl | | | | | | | | | |
| 265 | H | H | diethylamino | H | H | H | H | Cl | H | cyclohexyl | | 1.69[a] |
| 266 | H | H | acetyl | H | H | H | H | Cl | H | cyclohexyl | | 2.77[a] |
| 267 | H | H | methylsulphonyl | H | H | H | H | Cl | H | cyclohexyl | | 2.54[a] |
| 268 | H | H | fluoro | H | H | H | H | Cl | H | propan-2-yl | | |
| 269 | H | H | H | H | H | H | H | Cl | ethyl | propan-2-yl | | 3[b] |
| 270 | H | H | H | H | H | H | H | Cl | CH₃ | 2-methylpropyl | | 3[b] |
| 271 | H | H | H | H | H | H | H | Cl | CH₃ | prop-2-en-1-yl | | 4.03[c]; |
| | | | | | | | | | | | | 2.66[b] |
| 272 | H | H | H | H | H | H | H | Cl | CH₃ | propyl | | 4.35[c]; |
| | | | | | | | | | | | | 2.55[b] |
| 273 | H | H | H | H | H | H | H | Cl | H | 2,2-dimethylpropyl | | 2.48[b] |
| 274 | H | H | H | H | H | H | H | Cl | H | pentan-2-yl | | 2.41[b] |
| 275 | H | H | H | H | H | H | H | Cl | H | pentan-3-yl | | 2.34[b] |
| 276 | H | H | H | H | H | H | H | Cl | H | 2-methylbutyl | | 2.37[b] |
| 277 | H | H | H | H | H | H | H | Cl | H | 3-methylbutyl | | 2.37[b] |
| 278 | H | H | H | H | H | H | H | Cl | H | pentyl | | 2.41[b] |
| 279 | H | H | H | H | H | H | H | Cl | H | 2-methylpropyl | | 2.02[b] |
| 280 | H | H | H | H | H | H | H | Cl | H | propyl | | 1.69[b] |
| 281 | H | difluoromethyl | H | H | H | H | H | Cl | H | propan-2-yl | | 2.4[b] |
| 282 | H | methylsulphanyl | H | H | H | H | H | Cl | CH₃ | butyl | | 3.9[b] |
| 283 | H | methylsulphanyl | H | H | H | H | H | Cl | H | 2-methylpropyl | | 2.69[b] |
| 284 | H | methylsulphanyl | H | H | H | H | H | Cl | H | propyl | | 3.87[c]; |
| | | | | | | | | | | | | 2.3[b] |
| 285 | H | H | H | H | H | H | H | Cl | H | 2-hydroxyethyl | | 1.83[c]; |
| | | | | | | | | | | | | 0.7[b] |
| 286 | H | H | H | H | H | H | H | Cl | H | prop-2-en-1-yl | | 3.1[c]; |
| | | | | | | | | | | | | 1.64[b] |
| 287 | H | H | H | H | H | H | H | Cl | H | (trimethylsilyl)methyl | | 2.14[b] |
| 288 | H | H | H | H | H | H | H | Cl | H | 2-methylprop-2-en-1- | | 2.62[b] |
| | | | | | | | | | | yl | | |
| 289 | H | H | H | H | H | H | H | Cl | H | 3-methylbut-2-en-1- | | 2.17[b] |
| | | | | | | | | | | yl | | |
| 290 | H | methyl(propanoyl)- | H | H | H | H | H | Cl | H | propan-2-yl | | 2.89[c]; |
| | | amino | | | | | | | | | | 1.76[b] |
| 291 | H | H | H | H | H | H | H | Cl | H | 1-(trimethylsilyl)ethyl | | 2.66[b] |
| 292 | H | methylamino | H | H | H | H | H | Cl | H | propan-2-yl | | 1.32[b] |
| 293 | H | H | H | H | H | H | H | Cl | H | 2-(methylsulphanyl)- | | 1.75[b] |
| | | | | | | | | | | ethyl | | |
| 294 | H | H | H | H | H | H | H | Cl | H | but-3-en-1-yl | | 1.87[b] |
| 295 | H | H | H | H | H | H | H | Cl | H | 3-methoxybutan-2-yl | | 3.64[c]; |
| | | | | | | | | | | | | 2.04[b] |
| 296 | H | H | H | H | H | H | H | Cl | H | butan-2-yl | | 2.05[b] |
| 297 | H | H | H | H | H | H | H | CH₃ | H | tert-butyl | | 1.48[b] |
| 298 | H | 1H-pyrazol-1-ylmethyl | H | H | H | H | H | Cl | H | propan-2-yl | | 1.7[b] |

The compounds of the formula (I) listed in Table II below are likewise obtained by the methods described above.

(I)

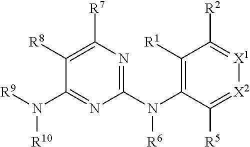

| Bsp | R⁵ | X² | X¹ = CR³ | R² | R¹ | R⁶ | R⁷ | R⁸ | R⁹ | R¹⁰ | HX | log p |
|---|---|---|---|---|---|---|---|---|---|---|---|---|
| II-1 | H | N | methoxy | H | H | H | H | Br | H | 1-methoxypropan-2- | | 1.53[a]; |
| | | | | | | | | | | yl | | 1.52[b] |
| II-2 | H | N | methoxy | H | H | H | H | Cl | H | 1-methoxypropan-2- | | 1.39[b] |
| | | | | | | | | | | yl | | |
| II-3 | H | N | cyclopropylamino | H | H | H | H | Cl | H | 1-methoxypropan-2- | | 1.17[b] |
| | | | | | | | | | | yl | | |
| II-4 | H | N | hydroxy | H | H | H | H | Br | H | 1-methoxypropan-2- | | 0.58[a] |
| | | | | | | | | | | yl | | |

The logP values were determined in accordance with EEC Directive 79/831 Annex V .A8 by HPLC (High Performance Liquid Chromatography) on reversed-phase columns (C 18), using the methods below:
[a] The determination was carried out in the acidic range at pH 2.3 using the mobile phases 0.1% aqueous phosphoric acid and acetonitrile linear gradient from 10% acetonitrile to 95% acetonitrile.
[b] The LC-MS determination in the acidic range was carried out at pH 2.7 using the mobile phases 0.1% aqueous formic acid and acetonitrile (contains 0.1% formic acid) linear gradient from 10% acetonitrile to 95% acetonitrile
[c] The LC-MS determination in the neutral range was carried out at pH 7.8 using the mobile phases 0.001 molar aqueous ammmonium bicarbonate solution and acetonitrile linear gradient from 10% acetonitrile to 95% acetonitrile
Calibration was carried out using unbranched alka-2-ones (having 3 to 16 carbon atoms), with known logP values (determination of the logP values by the retention times using linear interpolation between two successive alkanones).
The lambda-max values were determined in the maxima of the chromatographic signals uding the UV spectra from 200 nm to 400 nm.

| Example No. | | |
|---|---|---|
| from Table 1 | ¹H NMR | M + 1 |
| 1 | ¹H NMR (acetonitrile-d₃, pyrimidine-H): δ = 7.87 | 369.1 |
| 2 | ¹H NMR (acetonitrile-d₃, pyrimidine-H): δ = 7.91 | 313 |
| 3 | | 279.1 |
| 4 | | 339 |
| 5 | ¹H NMR (acetonitrile-d₃, pyrimidine-H): δ = 8.32 | 321.1 |
| 6 | ¹H NMR (acetonitrile-d₃): δ = 11.34 (br. s, 1H), 7.95 (s, 1 H), | 386.1 |
| | 7.87 (d, 2H), 7.78 (d, 2H), 3.75 (m, 2H), 3.62 (m, 2 H), 3.33 (s, | |
| | 3 H), 2.67 (s, 6H). | |
| 7 | ¹H NMR (acetonitrile-d₃, pyrimidine-H): δ = 7.85 | 463.2 |
| 8 | ¹H NMR (acetonitrile-d₃): δ = 10.46 (br. s, 1H), 7.85 (s, 1 H), | 351.1 |
| | 7.45 (d, 2H), 6.90 (d, 2H), 6.69 (br.s, 1H), 4.54 (m, 1H), | |
| | 4.37 (m, 1 H), 3.48 (m, 2 H), 3.31 s (3 H), 1.29 (d, 6H), 1.25 (d, 3H). | |
| 9 | | 335.1 |
| 10 | | 327.1 |
| 11 | ¹H NMR (acetonitrile-d₃, pyrimidine-H): δ = 7.85 | 321.2 |
| 12 | ¹H NMR (acetonitrile-d₃, pyrimidine-H): δ = 7.90 | 337.2 |
| 13 | ¹H NMR (acetonitrile-d₃, pyrimidine-H): δ = 7.83 | 364.1 |
| 14 | | 353.1 |
| 15 | | 335.1 |
| 16 | ¹H NMR: δ = 10.53 (br. s, 1H), 8.19 (s, 1 H), 7.91 (d, 2H), | 400.1 |
| | 7.75 (br. s, 1H), 7.69 (d, 2H), 4.48-4.46 (m, 1H), 3.55-3.52 (m, 1 | |
| | H), 3.40-3.37 (m, 1 H), 3.26 (s, 3H), 2.59 (s, 6 H), 1.22 (d, 3 | |
| | H). | |
| 17 | ¹H NMR (pyrimidine-H): δ = 8.03 | 327.1 |
| 18 | ¹H NMR (pyrimidine-H): δ = 8.06 | 293.1 |
| 19 | | 353.2 |
| 20 | ¹H NMR (pyrimidine-H): δ = 8.07 | 383.1 |
| 21 | | 335.2 |
| 22 | | 400.2 |
| 23 | | 477.3 |
| 24 | ¹H NMR (pyrimidine-H): δ = 8.16 | 417.2 |
| 25 | ¹H NMR (pyrimidine-H): δ = 8.18 | 327.1 |
| 26 | | 342 |
| 27 | | 356.1 |
| 28 | | 327.1 |
| 29 | | 442.1 |
| 30 | | 416.1 |
| 31 | | 400.1 |
| 32 | | 277.1 |
| 33 | | 249.1 |
| 34 | | 277.1 |
| 35 | | 235.1 |
| 36 | | 263.1 |
| 37 | | 309 |
| 38 | ¹H NMR: δ = 9.60 (br. s, 1H), 8.02 (s, 1 H), 7.86-7.81 (m, | 351.1 |
| | 4H), 6.76 (d, 1H), 4.45-4.42 (m, 1H), 3.81 (s, 3H), | |
| | 3.54-3.50 (m, 2 H), 3.41-3.38 (m, 2H), 1.22 (d, 3 H). | |
| 39 | ¹H NMR (pyrimidine-H): δ = 8.03 | 307.1 |
| 40 | ¹H NMR (acetonitrile-d₃, pyrimidine-H): δ = 7.95 | 291 |
| 41 | | 333.1 |
| 42 | | 291.1 |
| 43 | | 384.1 |
| 44 | ¹H NMR (pyrimidine-H): δ = 8.33 | 240 |
| 45 | | 319.2 |
| 46 | | 277.1 |
| 47 | | 370.1 |
| 48 | ¹H NMR: δ = 8.95 (br. s, 1H), 7.89 (s, 1 H), 7.67 (s, 1H), | 375.1 |
| | 7.54 (d, 1H), 7.18 (dd, 1 H), 6.77 (d, 1 H), 6.42 (d, 1 H), 4.30 (m, | |
| | 1H), 4.20 (s, 2H), 3.25 (m, 2 H), 3.14 (m, 2H), 2.67 (s, 3 H), | |
| | 1.23 (d, 6H). | |
| 49 | | 319.1 |
| 50 | | 305 |
| 51 | | 384 |
| 52 | ¹H NMR (pyrimidine-H): δ = 8.03 | 398.1 |
| 53 | | 265.1 |
| 54 | | 325 |
| 55 | | 355.1 |
| 56 | | 307.1 |
| 57 | | 372.1 |
| 58 | | 307.1 |
| 59 | | 323.2 |
| 60 | | 277.1 |
| 61 | ¹H NMR (acetonitrile-d₃, pyrimidine-H): δ = 7.88 | 279.1 |
| 62 | ¹H NMR (acetonitrile-d₃, pyrimidine-H): δ = 7.88 | 339 |
| 63 | | 442.1 |
| 64 | | 426.2 |
| 65 | | 277.1 |
| 66 | | 261.1 |
| 67 | ¹H NMR (pyrimidine-H): δ = 8.30 | 286.1 |
| 68 | ¹H NMR: δ = 9.52 (s, 1H), 8.29-8.28 (m, 1H), 7.76.7.71 (m | 254.2 |
| | 2H), 7.51 (br. s, 1H), 7.29-7.25 (m, 2H), 6.99-6.97 (m, 1H), | |
| | 3.4-3.35 (m, 2H), 1.65-1.58 8m, 2H), 0.93-0.88 (m, 3H) | |
| 69 | | 311 |
| 70 | ¹H NMR (pyrimidine-H): δ = 8.00 (s, 1 H) | 356 |
| 71 | ¹H NMR (pyrimidine-H): δ = 8.06 (s, 1 H) | 372 |
| 72 | ¹H NMR: δ = 9.45 (s, 1 H), 8.33 (s, 1 H), 8.06 (s, 1 H), | 445 |
| | 7.91 (dd, 1 H), 7.48 (t, 1 H), 7.25 (dd, 1 H), 6.28 (br. d, 1 H), | |
| | 4.49 (m, 1 H), 3.50 (dd, 1 H), 3.40 (dd, 1 H), 3.28 (s, 3 H), 2.64 (s, 6 | |
| | H), 1.22 (d, 3 H). | |
| 73 | | 372 |
| 74 | | 396.1 |
| 75 | | 466 |
| 76 | | 448 |
| 77 | | 382 |
| 78 | | 392.1 |
| 79 | | 419.1 |
| 80 | ¹H NMR (pyrimidine-H): δ = 7.98 (s, 1 H) | 395 |
| 81 | | 328.1 |
| 82 | | 360.1 |
| 83 | | 317.1 |
| 84 | | 352.1 |
| 85 | | 322.1 |
| 86 | | 322.1 |
| 87 | | 376.1 |
| 88 | | 392.1 |
| 89 | | 372.1 |
| 90 | | 375.1 |
| 91 | | 364.1 |
| 92 | | 422.1 |
| 93 | | 364.1 |
| 94 | | 360 |
| 95 | | 384.1 |
| 96 | | 336.1 |
| 97 | | 336.1 |
| 98 | | 410 |
| 99 | | 363.2 |
| 100 | | 404 |
| 101 | | 338.1 |
| 102 | | 359.1 |
| 103 | | 346.1 |
| 104 | | 348.1 |
| 105 | | 363.1 |
| 106 | | 366.1 |
| 107 | | 375.2 |
| 108 | | 361 |
| 109 | | 366.1 |
| 110 | | 366.1 |
| 111 | | 416 |
| 112 | | 419 |
| 113 | | 408.1 |
| 114 | | 404 |
| 115 | | 428 |
| 116 | | 380.1 |
| 117 | | 380 |
| 118 | | 454 |
| 119 | | 407.1 |
| 120 | | 485.1 |
| 121 | | 403.1 |
| 122 | | 407 |
| 123 | | 410 |
| 124 | | 393.1 |
| 125 | | 423.1 |
| 126 | | 455.1 |
| 127 | | 393.1 |
| 128 | | 419.1 |
| 129 | | 423.1 |
| 130 | | 409.1 |
| 131 | | 379.1 |
| 132 | | 379.1 |
| 133 | | 405.1 |
| 134 | | 394.1 |
| 135 | | 349.1 |
| 136 | | 375.2 |
| 137 | | 391.2 |
| 138 | | 335.1 |
| 139 | | 361.1 |
| 140 | | 379.1 |
| 141 | | 411.1 |
| 142 | | 349.1 |
| 143 | | 379.1 |
| 144 | | 365.1 |
| 145 | | 370.1 |
| 146 | | 391.1 |
| 147 | | 391.1 |
| 148 | | 363.1 |
| 149 | | 338.1 |
| 150 | | 439.1 |
| 151 | | 390.2 |
| 152 | | 334.1 |
| 153 | | 365.1 |
| 154 | | 352.1 |
| 155 | | 363.1 |
| 156 | | 377.2 |
| 157 | | 391.2 |
| 158 | | 408.1 |
| 159 | | 350.1 |
| 160 | | 326.1 |
| 161 | | 431.2 |
| 162 | | 389.1 |
| 163 | | |
| 164 | | 443.1 |
| 165 | | 427.1 |
| 166 | | 409.1 |
| 167 | | 363.1 |
| 168 | | 401.1 |
| 169 | | 377.1 |
| 170 | | 377.1 |
| 171 | | 322.1 |
| 172 | | 374.1 |
| 173 | ¹H NMR: δ = 8.99 (br. s, 1H), 7.91 (s, 1 H), 7.78 (s, 1H), | 309.1 |
| | 7.44 (d, 1H), 7.16 (dd, 1 H), 6.81 (d, 1 H), 6.49 (d, 1 H), 4.34 (m, | |
| | 1H), 2.44 (s, 3 H), 1.24 (d, 6 H). | |
| 174 | ¹H NMR (pyrimidine-H): δ = 7.94 | 323.1 |
| 175 | ¹H NMR (pyrimidine-H): δ = 7.96 | 369 |
| 176 | ¹H NMR (pyrimidine-H): δ = 7.94 | 321.1 |
| 177 | | 332.1 |
| 178 | | 292 |
| 179 | | 333.1 |
| 180 | | 288.1 |
| 181 | | 350.1 |
| 182 | | 293.1 |
| 183 | | 293.1 |
| 184 | | 288.1 |
| 185 | | 347.1 |
| 186 | | 341.1 |
| 187 | | 343.1 |
| 188 | | 362.2 |
| 189 | | 320.1 |
| 190 | | 354.1 |
| 191 | | 348.2 |
| 192 | | 334.1 |
| 193 | | 374.1 |
| 194 | | 360.1 |
| 195 | | 320.1 |
| 196 | | 346.1 |
| 197 | | 362.1 |
| 198 | | 410.1 |
| 199 | | 361.1 |
| 200 | | 305.1 |
| 201 | | 336.1 |
| 202 | | 350.1 |
| 203 | | 336.1 |
| 204 | | 336.1 |
| 205 | | 320.1 |
| 206 | | 346.1 |
| 207 | | 360.1 |
| 208 | | 334.2 |
| 209 | | 348.1 |
| 210 | | 346.1 |
| 211 | | 379 |
| 212 | | 345 |
| 213 | | 279.1 |
| 214 | | 279.1 |
| 215 | | 302.1 |
| 216 | | 350.1 |
| 217 | | 348.1 |
| 218 | | 392 |
| 219 | ¹H NMR: δ = 9.10 (br. s, 1H), 7.99 (s, 1 H), 7.71 (dd, 1H), | 323.1 |
| | 7.42 (dd, 1H), 7.18 (dd, 1 H), 6.83 (dd, 1 H), 4.73 (m, 1 H), 2.98 (s, | |
| | 3H), 2.44 (s, 3 H), 1.21 (d, 6 H). | |
| 220 | ¹H NMR (pyrimidine-H): δ = 8.05 | 260 |
| 221 | ¹H NMR (pyrimidine-H): δ = 8.06 | 306.1 |
| 222 | ¹H NMR (pyrimidine-H): δ = 7.96 (s, 1 H). | 293 |
| 223 | ¹H NMR (pyrimidine-H): δ = 7.91 (s, 1 H). | 279 |
| 224 | ¹H NMR (pyrimidine-H): δ = 8.01 | 367.1 |
| 225 | ¹H NMR (pyrimidine-H): δ = 7.91 | 307.1 |
| 226 | ¹H NMR: δ = 8.95 (s, 1 H), 7.97 (s, 1 H), 7.67 (dd, 2 H), | 366 |
| | 7.19-7.29 (m, 2 H), 6.87-6.93 (m, 1 H), 6.08 (d, 1 H), 4.22-4.29 (m, 1 | |
| | H), 3.28-3.51 (m, 2 H), 3.27 (s, 3 H), 1.56-1.66 (m, 2 H), | |
| | 1.24-1.38 (m, 2 H), 0.86-0.92 (m, 3 H). | |
| 227 | | 404.1 |
| 228 | ¹H NMR (pyrimidine-H): δ = 7.97 (s, 1 H). | 350 |
| 229 | ¹H NMR: δ = 8.84 (br. s, 1H), 7.89 (s, 1 H), 7.61 (s, 1H), | 277.1 |
| | 7.42 (d, 1H), 7.09 (dd, 1 H), 6.71 (d, 1 H), 6.42 (d, 1 H), 4.30 (m, | |
| | 1H), 2.26 (s, 3 H), 1.24 (d, 6H). | |
| 230 | ¹H NMR: δ = 8.84 (br. s, 1H), 7.89 (s, 1 H), 7.65 (s, 1H), | 291.1 |
| | 7.42 (d, 1H), 7.12 (dd, 1 H), 6.74 (d, 1 H), 6.43 (d, 1 H), 4.34 (m, | |
| | 1H), 2.55 (q, 2 H), 1.24 (d, 6H), 1.53 (t, 3 H). | |
| 231 | ¹H NMR (pyrimidine-H): δ = 8.07 (s, 1 H). | 380 |
| 232 | ¹H NMR: δ = 9.01 (s, 1 H), 8.09 (s, 1 H), 7.65 (dd, 2 H), | 366 |
| | 7.22 (dd, 2 H), 6.89 (dd, 1 H), 4.65-4.69 (m, 1 H), 3.42-3.59 (m, 2 | |
| | H), 3.25 (s, 3 H), 2.99 (s, 3H), 1.61-1.68 (m, 2 H), | |
| | 0.86-0.89 (m, 3 H). | |
| 233 | ¹H NMR (pyrimidine-H): δ = 8.09 (s, 1 H) | 364/366 |
| 234 | ¹H NMR: δ = 9.16 (br. s, 1H), 7.93 (s, 1 H), 7.75 (d, 2H), | 334.1 |
| | 7.30 (d, 2H), 6.50 (d, 1 H), 4.33 (m, 1H), 2.96 (s, 3 H), 1.24 (d, 6H). | |
| 235 | ¹H NMR (pyrimidine-H): δ = 7.88 | 274.1 |
| 236 | ¹H NMR: δ = 9.07 (br. s, 1H), 7.91 (s, 1 H), 7.85 (s, 1H), | 302.1 |
| | 7.55 (d, 1H), 7.23 (dd, 1 H), 6.86 (d, 1 H), 6.47 (d, 1 H), 4.36 (m, | |
| | 1H), 3.92 (s, 2H), 1.24 (d, 6H). | |
| 237 | ¹H NMR (pyrimidine-H): δ = 8.09 (s, 1 H). | 352 |
| 238 | ¹H NMR (pyrimidine-H): δ = 7.90 | 295 |
| 239 | ¹H NMR: δ = 9.05 (br. s, 1H), 7.97 (s, 1 H), 7.71 (s, 1H), | 309.1 |
| | 7.44 (d, 1H), 7.18 (dd, 1 H), 6.81 (d, 1 H), 3.65 (q, 2H), 3.15 (s, 3 | |
| | H), 2.44 (s, 3 H), 1.20 (t, 3H). | |
| 240 | ¹H NMR (pyrimidine-H): δ = 7.89 | 249.1 |
| 241 | ¹H NMR (pyrimidine-H): δ = 7.96 | 263.1 |
| 242 | ¹H NMR (pyrimidine-H): δ = 7.93 | 412.1 |
| 243 | ¹H NMR (pyrimidine-H): δ = 7.90 | 398.1 |
| 244 | ¹H NMR (acetonitrile-d₃): δ = 9.96 (s, 1H), 8.38 (s, 1H), | 321.1 |
| | 7.94 (s, 1H), 7.92-7.75 (m, 2H), 7.55-7.45 (m, 2H), 5.45 (s, 1H), | |
| | 4.51-4.39 (1H, s), 3.55-3.42 (m, 2H), 3.33 (s, 3H), 1.27 (d, 3H) | |
| 245 | ¹H NMR: δ = 9.18 (br. s., 1H), 8.02 (s, 1H), 7.76-7.74 (m, 2H), | 380.1 |
| | 7.31-7.30 (br. d 2H), 4.32-4.30 (m, 1H), 2.96 (s, 6H), | |
| | 1.26-1.24 (m, 6H) | |
| 246 | ¹H NMR (pyrimidine-H): δ = 8.01 | 346 |
| 247 | ¹H NMR: δ = 8.93 (br. s, 1H), 7.99 (s, 1 H), 7.68 (d, 2H), | 307 |
| | 7.22 (dd, 2H), 6.89 (t, 1 H), 6.14 (d, 1 H), 4.31 (m, 1H), 1.24 (d, 6H). | |
| 248 | ¹H NMR: δ = 8.95 (br. s, 1H), 7.89 (s, 1 H), 7.71 (s, 1H), | 460.1 |
| | 7.59 (d, 1H), 7.48 (d, 1 H), 7.33 (m, 4 H), 7.21 (m, 2 H), 6.78 (d, 1 | |
| | H), 6.42 (d, 1H), 4.44 (m 1 H), 4.31 (m, 1H), 4.00 (m, 2 H), | |
| | 1.39 (d, 3 H), 1.23 (d, 6H). | |
| 249 | | 345 |
| 250 | ¹H NMR: δ = 9.04 (br. s, 1H), 7.91 (s, 1 H), 7.86 (s, 1H), | 384.1 |
| | 7.61 (d, 1H), 7.22 (dd, 1 H), 6.94 (d, 1 H), 6.45 (d, 1H), 4.37 (m 1 | |
| | H), 4.25 (s, 2 H), 2.74 (s, 6 H), 1.24 (d, 6H). | |
| 251 | ¹H NMR: δ = 9.78 (br.s, 1 H), 8.04 (s, 1 H), 7.70 (s, 1H), | 446.1 |
| | 7.56 (d, 1H), 7.48 (d, 1 H), 7.43 (br.s, 1 H), 7.30 (m, 5 H), 7.02 (d | |
| | 1H), 4.37 (m 1 H), 4.25 (s, 2H), 4.10 (d, 2 H), 1.23 (d, 6H). | |
| 252 | ¹H NMR: δ = 9.95 (s, 1H), 9.28 (s, 1H), 8.42 (s, 1H), 7.95 (s, | 291.1 |
| | 1H), 7.91-7.86 (m, 1H), 7.48-7.43 (m, 2H), 6.53 (d, 1H), | |
| | 4.43-4.30 (m, 1H), 1.25 (d, 6H) | |
| 253 | | 332 |
| 254 | ¹H NMR: δ = 8.93 (s, 1H), 7.90 (s, 1H), 7.72.7.70 (m 2H), | 289.1 |
| | 7.24-7.20 (m, 2H), 6.91-6.89 (m, 1H), 6.52-6.50 (d, 1H), | |
| | 4.40-4.36 (m, 1H), 2.04-1.98 (m, 2H), 1.73-1.69 (m, 2H), 1.62-1.58 (m, | |
| | 4H) | |
| 255 | ¹H NMR: δ = 8.94 (br. S, 1H), 7.89 (s, 1H), 7.66 (d, 1H), | 345.1 |
| | 7.53-7.51 (q. 1H), 7.19-7.15 (m, 1H), 6.76-6.74 (m, 1H), | |
| | 6.51-6.49 (m, 1H), 4.40-4.38 (m, 1H), 3.57-3.55 (s, 3H), 2.10 (s, 2H), | |
| | 1.99-1.96 (m, 2H), 1.73-1.70 (m, 2H), 1.61-1.57 (m, 4H), | |
| 256 | ¹H NMR: δ = 8.98 (s, 1H), 7.91 8s, 1H), 7.83-7.82 (d, 1H), | 335.1 |
| | 7.44-7.42 (m 1H), 7.18-7.16 (q, 1H), 6.82-6.80 (m, 1H), | |
| | 6.55-6.50 (m, 1H), 4.42-4.39 (m, 1H), 2.49-2.48 (s, 3H), | |
| | 2.04-2.00 (m, 2H), 1.73-1.70 (m, 2H) | |
| 257 | ¹H NMR (pyrimidine-H): δ = 8.15 | 365.2 |
| 258 | | 337 |
| 259 | | 303 |
| 260 | | 363 |
| 261 | | 393 |
| 262 | | 345 |
| 263 | | 410 |
| 264 | | 487.2 |
| 265 | ¹H NMR (pyrimidine-H): δ = 7.88 | 374.2 |
| 266 | ¹H NMR (pyrimidine-H): δ = 8.06 | 345.2 |
| 267 | | 381.1 |
| 269 | | 291.3 |
| 270 | | 291.3 |
| 271 | ¹H NMR (pyrimidine-H): δ = 7.99 | 275.1 |
| 272 | ¹H NMR (pyrimidine-H): δ = 7.95 | 277.1 |
| 273 | | 291.3 |
| 274 | | 291.3 |
| 275 | | 291.3 |
| 276 | | 291.3 |
| 277 | | 291.3 |
| 278 | | 291.4 |
| 279 | | 277.3 |
| 280 | | 263.3 |
| 281 | ¹H NMR: δ = 9.20 (s, 1H), 8.14 (s, 1H), 7.94 (s, 1H), | 313.1 |
| | 7.72-7.71 (q, 1H), 7.37.7.33 (m, 1H), 7.07-7.05 (m, 1H), 6.50 (d, 1H), | |
| | 4.36-4.34 (m, 1H), 1.25-1.23 (q, 6H) | |
| 282 | ¹H NMR (pyrimidine-H): δ = 7.94 | 337.2 |
| 283 | | 323.4 |
| 284 | | 309.4 |
| 285 | ¹H NMR (pyrimidine-H): δ = 7.91 | 265.1 |
| 286 | ¹H NMR: δ = 8.92 (s, 1H), 7.92 (s, 1 H), 7.67 (d, 2H), 7.22 (dd, | 261.2 |
| | 2H), 7.11 (br.s, 1H), 6.88 (dd, 1 H), 5.94 (m, 1H), 5.15 (m, 2 | |
| | H), 4.05 (m, 2 H). | |
| 287 | | 307.2 |
| 288 | | 275.2 |
| 289 | | 289.2 |
| 291 | | 321.2 |
| 293 | | 295.2 |
| 294 | | |
| 294 | | 275.2 |
| 295 | ¹H NMR (pyrimidine-H): δ = 7.93 | 307.1 |
| 296 | ¹H NMR: δ = 8.90 (s, 1H), 7.89 (s, 1H), 7.70-7.67 (m, 2H), | 277.1 |
| | 7.24-7.20 (m, 2H), 6.89-6.86 (m, 1H), 4.15-4.11 (m, 1H), | |
| | 1.68-1.63 (m, 1H), 1.57-1.52 (m, 1H), 1.21-1.19 (q, 3H), | |
| | 0.91-0.87 (m, 3H) | |
| 297 | ¹H NMR (pyrimidine-H): δ = 8.42 | 257.2 |
| 298 | ¹H NMR: δ = 9.19 (s, 1H), 7.91 (s, 1H), 7.75-7.73 (M, 2H), | 343.1 |
| | 7.55-7.53 (m, 1H), 7.44-7.43 (s, 1H), 7.20-7.16 (m, 1H), | |
| | 6.74-6.70 (m, 2H), 6.26-6.25 (d, 1H), 5.25 (d, 2H), 4.33-4.19 (m, | |
| | 1H), 1.23-1.21 (q, 6H) | |
| 290 | ¹H NMR: δ = 9.34 (br. s, 1H), 7.95 (s, 1 H), 7.86 (s, 1H), | 348.2 |
| | 7.52 (d, 1H), 7.29 (dd, 1 H), 6.81 (m, 2 H), 4.29 (m, 1H), 3.13 (s, 3 | |
| | H), 2.06 (m, 2 H), 1.21 (d, 6H), 0.92 (t, 3H). | |
| 292 | ¹H NMR: δ = 8.85 (s, 1H), 7.88 (s, 1 H), 7.03 (s, 1H), 6.91 (dd, | 292.2 |
| | 1H), 6.84 (d, 1 H), 6.62 (d, 1 H), 6.14 (d, 1H), 5.40 (s, 1 H), | |
| | 4.39 (m, 1 H), 2.67 (m, 3 H), 1.21 (d, 6H). | |
| Ia-1 | ¹H NMR: δ = 8.90 (s, 1 H), 8.38 (d, 1 H), 7.98 (s, 1 H), | 369 |
| | 7.95 (dd, 1 H), 6.72 (d, 1 H), 6.12 (br. d, 1 H), 4.34 (m, 1 H), 3.81 (s, | |
| | 3 H), 3.48 (dd, 1 H), 3.37 (dd, 1 H), 3.28 (s, 3 H), 1.20 (d, 3 H). | |
| Ia-2 | ¹H NMR: δ = 8.89 (s, 1 H), 8.39 (d, 1 H), 7.96 (dd, 1 H), | 324 |
| | 7.89 (s, 1 H), 6.70 (d, 1 H), 6.40 (br. d, 1 H), 4.36 (m, 1 H), 3.81 (s, 3 | |
| | H), 3.48 (dd, 1 H), 3.36 (dd, 1 H), 3.28 (s, 3 H), 1.20 (d, 3 H). | |
| Ia-3 | | 348.1 |
| Ia-4 | ¹H NMR: δ = 8.57 (s, 1 H), 7.94 (s, 1 H), 7.70 (d, 1 H), | 355 |
| | 7.58 (dd, 1 H), 6.28 (d, 1 H), 6.09 (br. d, 1 H), 4.30 (m, 1 H), | |
| | 3.46 (dd, 1 H), 3.37 (dd, 1 H), 3.22 (br. s, 1 H), 1.17 (d, 3 H). | |

The chemical NMR shifts were measured in ppm at 400 MHz, unless indicated otherwise in the solvent DMSO-d₆ using tetramethylsilane as internal standard.

The following abbreviations describe signal splitting:

s=singlet, d=doublet, t=triplet, q=quadruplet, m=multiplet

### EXAMPLE A

### Venturia Test (Apple)/Protective

Solvents: 24.5 parts by weight of acetone
24.5 parts by weight of dimethylacetamide
   Emulsifier: 1 part by weight of alkylaryl polyglycol ether

To produce a suitable preparation of active compound, 1 part by weight of active compound is mixed with the stated amounts of solvents and emulsifier, and the concentrate is diluted with water to the desired concentration.

To test for protective activity, young plants are sprayed with the preparation of active compound at the stated application rate. After the spray coating has dried on, the plants are inoculated with an aqueous conidia suspension of the apple scab pathogen Venturia inaequalis and then remain in an incubation cabinet at about 20° C. and 100% relative atmospheric humidity for 1 day.

The plants are then placed in a greenhouse at about 21° C. and a relative atmospheric humidity of about 90%.

Evaluation is carried out 10 ten days after the inoculation. 0% means an efficacy which corresponds to that of the control, whereas an efficacy of 100% means that no infection is observed.

In this test, the Examples Nos. 4, 25, 36, 77, 84, 93, 96, 109, 113, 127, 128, 130, 135, 136, 137, 144, 151, 183, 189, 191, 192, 199, 202, 203, 204, 214, 245, 249 and 250 from Table I showed, at an active compound concentration of 100 ppm, an efficacy of 70% or more.

### EXAMPLE B

### Alternaria Test (Tomato)/Protective

Solvent: 49 parts by weight of N,N-dimethylformamide
Emulsifier: 1 part by weight of alkylaryl polyglycol ether

To produce a suitable preparation of active compound, 1 part by weight of active compound is mixed with the stated amounts of solvent and emulsifier, and the concentrate is diluted with water to the desired concentration.

To test for protective activity, young tomato plants are sprayed with the preparation of active compound at the stated application rate. 1 day after the treatment, the plants are inoculated with a spore suspension of Alternaria solani and then remain at 100% rel. humidity and 22° C. for 24 h. The plants then remain at 96% rel. atmospheric humidity and a temperature of 20° C.

Evaluation is carried out 7 days after the inoculation. 0% means an efficacy which corresponds to that of the control, whereas an efficacy of 100% means that no infection is observed.

In this test, the Examples Nos. 3, 4, 29, 48, 72, 74, 80, 83, 85, 93, 96, 107, 109, 110, 113, 119, 121, 122, 125, 126, 128, 129, 130, 131, 132, 135, 136, 137, 138, 139, 140, 144, 188, 189, 190, 191, 192, 193, 195, 198, 199, 203, 204, 207, 212, 213, 214, 232, 234, 236, 238, 242, 243, 245, 248, 227, 249, 250, 255 and 262 from Table I showed, at an active compound concentration of 500 ppm, an efficacy of 70% or more.

### EXAMPLE C

### Pyrenophora teres Test (Barley)/Protective

Solvent: 49 parts by weight of N,N-dimethylformamide
Emulsifier: 1 part by weight of alkylaryl polyglycol ether

To produce a suitable preparation of active compound, 1 part by weight of active compound is mixed with the stated amounts of solvent and emulsifier, and the concentrate is diluted with water to the desired concentration.

To test for protective activity, young barley plants are sprayed with the preparation of active compound at the stated application rate. 1 day after the treatment, the plants are inoculated with an aqueous spore suspension of Pyrenophora teres and then remain at 100% rel. atmospheric humidity and 22° C. for 48 h. The plants are then placed in a greenhouse at 80% rel. atmospheric humidity and a temperature of 20° C.

Evaluation is carried out 7-9 days after the inoculation. 0% means an efficacy which corresponds to that of the control, whereas an efficacy of 100% means that no infection is observed.

In this test, the Examples Nos. 4, 25, 47, 50, 62, 65, 66, 69, 70, 72, 74, 79, 84, 85, 86, 96, 99, 101, 103, 109, 110, 116, 121, 122, 124, 125, 128, 129, 130, 131, 132, 135, 136, 138, 140, 143, 144, 145, 152, 153, 165, 166, 182, 183, 186, 188, 189, 192, 195, 196, 197, 199, 203, 204, 213, 214, 216, 229, 230, 232, 234, 236, 238, 239, 240, 241, 242, 243, 245, 246, 247, 250, 252, 253, 254, 256, 281, 283, 284, 285 and 286 from Table I and the Examples II-1 and II-2 from Table II showed, at an active compound concentration of 500 ppm, an efficacy of 70% or more.

### EXAMPLE D

### Fusarium nivale (Var. majus) Test (Wheat)/Protective

Solvent: 50 parts by weight of N,N-dimethylacetamide
Emulsifier: 1 part by weight of alkylaryl polyglycol ether

To produce a suitable preparation of active compound, 1 part by weight of active compound is mixed with the stated amounts of solvent and emulsifier, and the concentrate is diluted with water to the desired concentration.

To test for protective activity, young plants are sprayed with the preparation of active compound at the stated application rate. After the spray coating has dried on, the plants are sprayed with spores of a spore suspension of Fusarium nivale (var. majus). The plants are placed in a greenhouse chamber under a translucent incubation hood at 10° C. and 100% relative atmospheric humidity.

Evaluation is carried out 5 days after the inoculation. 0% means an efficacy which corresponds to that of the control, whereas an efficacy of 100% means that no infection is observed.

In this test, the Examples Nos. 25, 69, 70, 74, 84, 96, 110, 125, 129, 130, 135, 144, 173, 234, 245 and 256 from Table I and Example II-1 from Table II showed, at an active compound concentration of 1000 ppm, an efficacy of 70% or more.

### EXAMPLE E

### Pyricularia Test (Rice)/Protective

Solvent: 28.5 parts by weight of acetone
Emulsifier: 1.5 parts by weight of alkylaryl polyglycol ether

To produce a suitable preparation of active compound, 1 part by weight of active compound is mixed with the stated amount of solvent, and the concentrate is diluted with water and the stated amount of emulsifier to the desired concentration.

To test for protective activity, young rice plants are sprayed with the preparation of active compound at the stated application rate. 1 day after the treatment, the plants are inoculated with an aqueous sprore suspension of Pyricularia oryza. The plants are then placed in a greenhouse at 100% relative atmospheric humidity and 25° C.

Evaluation is carried out 5 days after the inoculation. 0% means an, efficacy which corresponds to that of the control, whereas an efficacy of 100% means that no infection is observed.

In this test, the compounds according to the invention Nos. 229, 236, 245, 250, 252 and 255 from Table I showed, at an active compound concentration of 250 ppm, an efficacy of 80% or more.

### EXAMPLE F

### Rhizoctonia Test (Rice)/Protective

Solvent: 28.5 parts by weight of acetone
Emulsifier: 1.5 parts by weight of alkylaryl polyglycol ether

To produce a suitable preparation of active compound, 1 part by weight of active compound is mixed with the stated amount of solvent, and the concentrate is diluted with water and the stated amount of emulsifier to the desired concentration.

To test for protective activity, young rice plants are sprayed with the preparation of active compound at the stated application rate. 1 day after the treatment, the plants are inoculated with hypha of Rhizoctonia solani. The plants are then placed in a greenhouse at 100% relative atmospheric humidity and 25° C.

Evaluation is carried out 4 days after the inoculation. 0% means an efficacy which corresponds to that of the control, whereas an efficacy of 100% means that no infection is observed.

In this test, the Examples Nos. 144, 245, 250 and 255 from Table I showed, at an active compound concentration of 250 ppm, an efficacy of 80% or more.

### EXAMPLE G

### Cochliobolus Test (Rice)/Protective

Solvent: 28.5 parts by weight of acetone
Emulsifier: 1.5 parts by weight of alkylaryl polyglycol ether

To produce a suitable preparation of active compound, 1 part by weight of active compound is mixed with the stated amount of solvent, and the concentrate is diluted with water and the stated amount of emulsifier to the desired concentration.

To test for protective activity, young rice plants are sprayed with the preparation of active compound at the stated application rate. 1 day after the treatment, the plants are inoculated with an aqueous sprore suspension of Cochliobolus miyabeanus. The plants are then placed in a greenhouse at 100% relative atmospheric humidity and 25° C.

Evaluation is carried out 4 days after the inoculation. 0% means an efficacy which corresponds to that of the control, whereas an efficacy of 100% means that no infection is observed.

In this test, the Examples Nos. 236 and 255 from Table I showed, at an active compound concentration of 250 ppm, an efficacy of 80% or more.

### EXAMPLE H

### Gibberella Test (Rice)/Protective

Solvent: 28.5 parts by weight of acetone
Emulsifier: 1.5 parts by weight of alkylaryl polyglycol ether

To produce a suitable preparation of active compound, 1 part by weight of active compound is mixed with the stated amount of solvent, and the concentrate is diluted with water and the stated amount of emulsifier to the desired concentration.

To test for protective activity, young rice plants are sprayed with the preparation of active compound at the stated application rate. 1 day after the treatment, the plants are inoculated with an aqueous sprore suspension of Gibberella zeae. The plants are then placed in a greenhouse at 100% relative atmospheric humidity and 25° C.

Evaluation is carried out 5 days after the inoculation. 0% means an efficacy which corresponds to that of the control, whereas an efficacy of 100% means that no infection is observed.

In this test, Example No. 173 from Table I showed, at an active compound concentration of 250 ppm, an efficacy of 80% or more.

### EXAMPLE J

### Production of Fumonisin FBI by Fusarium proliferatum

The method used was adapted to microtitre plates using the method described by Lopez-Errasquin et al.: Journal of Microbiological Methods 68 (2007) 312-317.

Fumonisin-inducing liquid medium (Jiménez et al., Int. J. Food Microbiol. (2003), 89, 185-193) was inoculated with a concentrated spore suspension of Fusarium proliferatum (350 000 spores/ml, stored at −160° C.) to a final concentration of 2000 spores/ml.

The compounds were dissolved (10 mM in 100% DMSO) and diluted to 100 μM in H₂O. The compounds were tested at 7 concentrations in a range of from 50 μM to 0.01 μM (diluted, starting with the 100 μM stock solution in 10% DMSO).

From each diluted solution, 5 μl were mixed with 95 μl of inoculated medium in a well of a 96-well microarray plate. The plate was covered and incubated at 20° C. for 6 days.

At the beginning and after 6 days, an OD measurement (OD620 multiple read per well (square: 3×3)) was carried out to calculate the “pI50” growth.

After 6 days, a sample of the liquid medium was diluted in 10% acetonitrile. The concentration of FB1 in the diluted samples was analysed by HPLC-MS/MS, and the results were used to calculate the “pI50 FB1” values.

HPLC-MS/MS was carried out using the parameters below:

Mass spectrometry instrument: Applied Biosystems API4000 QTrap

### HPLC: Agilent 1100

### Autosampler: CTC HTS PAL

Chromatography column: Waters Atlantis T3 (50×2 mm)
Examples of the Measured pI50 Values
Production of Fumortisin FB1 by Fusarium proliferatum

| Example No. | pI50 Fum | pI50 growth |
|---|---|---|
| 51 | 5.2 | <4.3 |
| 74 | 4.6 | <4.3 |
| 109 | 4.7 | <4.3 |
| 136 | 4.4 | <4.3 |
| 152 | 5 | <4.3 |
| 173 | 4.8 | <4.3 |
| 199 | 5.4 | 4.9 |
| 203 | 5.2 | 4.6 |
| 214 | 6 | <4.3 |
| 228 | 5.1 | <4.3 |
| 229 | 5.2 | 4.8 |
| 230 | 5.1 | 5.2 |
| 234 | 5.3 | 4.6 |
| 236 | 5.7 | 5.7 |

### EXAMPLE K

### Production of DON/Acetyl-DON by Fusarium graminearum

The compounds were tested in microtitre plates at 7 concentrations of from 0.07 μM to 50 μM in a DON-inducing liquid medium (1 g of (NH₄)₂HPO₄, 0.2 g of MgSO₄×7H₂O, 3 g of KH₂PO₄, 10 g of glycerol, 5 g of NaCl and 40 g of sucrose per litre) with oat extract (10%) and DMSO (0.5%). Inoculation was carried out using a concentrated spore suspension of Fusarium graminearum at a final concentration of 2000 spores/ml.

The plate was incubated at high atmospheric humidity at 28° C. for 7 days.

At the beginning and after 3 days, an OD measurement was carried out at OD520 (repeated measurements: 3×3 measurements per well) to calculate the growth inhibition.

After 7 days, 100 μl of an 84/16 acetonitrile/water mixture were added, and samples of the liquid medium were then taken from each well and diluted 1:100 in 10% acetonitrile. The proportions of DON and acetyl-DON in the samples were analysed by HPLC-MS/MS, and the measured values were used to calculate the inhibition of the DON/AcDON production compared to an active compound-free control.

The HPLC-MS/MS measurements were carried out using the parameters below:

Ionization type: ESI negative
Ion spray voltage: −4500 V
Spray gas temperature: 500° C.
Decluster potential: −40 V
Collision energy: −22 eV

### Collision gas: N₂

NMR trace: 355.0>264.9;

HPLC column: Water, Atlantis T3 (trifunctional C18 bondung, capped)

Particle size: 3 μm
Column dimensions: 50×2 mm

### Temperature: 40° C.

Solvent A: water/2.5 mM NH₄OAc+0.05% CH₃COOH (v/v)
Solvent B: methanol/2.5 mM NH₄OAc+0.05% CH₃COOH (v/v)
Flow rate: 400 μl/minute
Injection volume: 11 μl

### Gradient:

| Time [min] | A % | B % |
|---|---|---|
| 0 | 100 | 0 |
| 0.75 | 100 | 0 |
| 1.5 | 5 | 95 |
| 4 | 5 | 95 |
| 5 | 100 | 0 |
| 10 | 100 | 0 |

### Example of DON Inhibition

Example No. 230 from Table I showed an inhibitory activity of >80% of DON/AcDON at 50 μM. At 86%, the growth inhibition of Fusarium graminearum of Example No. 230 at 50 μM was greater than 80%.

1. A method for controlling phytopathogenic harmful fungi comprising applying a compound of formula (I) or an agrochemically active salt thereof, 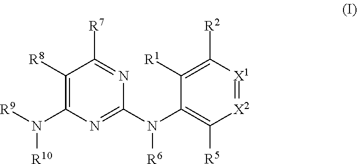
in which the symbols are as defined below:
X¹ represents nitrogen or CR³,
X² represents nitrogen or CR⁴,
where X¹ and X² do not both represent nitrogen,
R¹ and R⁵ independently of one another represent hydrogen, C₁-C₄-alkyl, C₁-C₄-alkoxy or halogen,
R² represents hydrogen, halogen, nitro, cyano, C₁-C₄-alkyl, C₁-C₂-haloalkyl, hydroxyl, OR¹², OCON(R¹²)₂, COR¹², CR¹²═NOR¹², SF₅, SR¹², SOR¹², SO₂R¹², CO₂R¹², N(R¹²)₂, NR¹²COR¹² or NR¹²CO₂R¹³,
R³ and R⁴ independently of one another represent hydrogen, halogen, cyano, nitro, a 3- to 8-membered, unsubstituted or substituted, saturated or unsaturated monocycle which contains no or up to four heteroatoms selected from the group consisting of N, O and S, where two oxygen atoms are not adjacent, OR¹², OCON(R¹²)₂, OCOR¹³, SF₅, SR¹², SOR¹², SO₂R¹², SO₂N(R¹³)₂, C═OR¹², CO(CH₂)ₘCN, CR¹²═NOR¹², CON(R¹²)₂, COOR¹², NR¹²COR¹², NR¹²CSR¹³, NR¹²COOR¹³, NR¹²(C═S)OR¹³, N(R¹²)₂, NR¹²SO₂R¹³, [C(R¹²)₂]ₘCN, (CH₂)ₘSON(R¹²)₂, (CH₂)ₘSO₂N(R¹²)₂, (CR¹²)ₘSO₂N(R¹²)₂, (CH₂)ₘCOOR¹², (CH₂)ₘCOR¹², [C(R¹²)₂]ₘCOR¹², (CH₂)ₘCON(R¹²)₂, [C(R¹²)₂]ₘCON(R¹²)₂, (CH₂)ₘNR¹²COR¹², (CH₂)ₘNR(¹²)₂, (CH₂)ₘNR¹²COOR¹³, [C(R¹²)₂]ₘNR¹²COR¹², [C(R¹²)₂]ₘNR¹²COOR¹³, unsubstituted or substituted C₁-C₈-alkyl, C₂-C₆-alkenyl, C₁-C₈-haloalkyl; where m=1-4,
where additionally R³ and R⁴, if appropriate via R¹² and R¹³, together may form a 5- to 7-membered, unsubstituted or substituted, saturated or unsaturated monocycle which may contain no or up to four heteroatoms selected from the group consisting of N, O and S, where two oxygen atoms are not adjacent,
where the substituents independently of one another are selected from the group consisting of hydrogen, fluorine, chlorine and bromine, C₁-C₄-alkyl, C₁-C₄-alkoxy, hydroxyl, oxo, (C₁-C₄-alkyl)carbonyl and cyano,
R⁶ represents hydrogen, C₁-C₃-alkyl, C₁-C₄-alkoxy-(C₁-C₄)alkyl, formyl, (C₁-C₄-alkyl)carbonyl, (C₁-C₄-alkoxy-C₁-C₄-alkyl)carbonyl, (C₃-C₆-alkenyloxy)carbonyl, (C₃-C₆-cycloalkyl)carbonyl, (halo-C₁-C₄-alkoxy-C₁-C₄-alkyl)carbonyl, (C₁-C₄-haloalkyl)carbonyl, (C₁-C₄-alkoxy)carbonyl, (C₁-C₄-haloalkoxy)carbonyl, benzyloxycarbonyl, unsubstituted or substituted benzyl, unsubstituted or substituted benzoyl, unsubstituted or substituted C₂-C₆-alkenyl, unsubstituted or substituted C₂-C₆-alkynyl, C₁-C₂-alkylsulphinyl or C₁-C₂-alkylsulphonyl,
where the substituents independently of one another are selected from the group consisting of hydrogen, fluorine, chlorine and bromine, C₁-C₄-alkyl, C₁-C₄-alkoxy, hydroxyl, C₁-C₄-haloalkyl and cyano,
R⁷ represents hydrogen, C₁-C₃-alkyl, cyano or C₁-C₃-haloalkyl,
R⁸ represents chlorine, bromine, iodine, cyano, methyl, SMe, SOMe, SO₂Me, CF₃, CCl₃, CFH₂ or CF₂H,
R⁹ represents hydrogen, C₁-C₃-alkyl, C₁-C₄-alkoxy-(C₁-C₄)alkyl, (C₁-C₄-alkyl)carbonyl, (C₁-C₄-haloalkyl)carbonyl, (C₁-C₄-alkoxy)carbonyl or optionally fluorine-, chlorine-, bromine-, cyano- or CF₃-substituted C₂-C₄-alkenyl,
R¹⁰ represents a group E1 of the formula 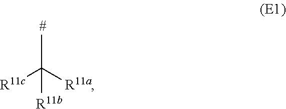
in which the symbols are as defined below and # represents the point of attachment to the nitrogen atom:
R^{11a} represents hydrogen or methyl,
R^{11b} represents hydrogen or C₁-C₃-alkyl,
R^{11c} represents hydrogen, C₁-C₄-alkyl, CN, CH₂CN, CH(Me)CN, C(Me)₂CN, CH₂OH, CH₂O—(C₁-C₃-alkyl), CH₂O—(C₁-C₃-haloalkyl), C(Me)HOH, C(Me)₂OH, C(Me)HO—(C₁-C₃-alkyl), C(Me)₂O—(C₁-C₃-alkyl), C(Me)HO—(C₁-C₃-haloalkyl), C(Me)₂O—(C₁-C₃-haloalkyl), CH₂S—(C₁-C₃-alkyl), C(Me)HS—(C₁-C₃-alkyl), C(Me)₂S—(C₁-C₃-alkyl), Si—(C₁-C₂-alkyl)₃ or C₂-C₄-alkenyl which is optionally substituted by halogen or CF₃;
or
R¹⁰ represents a cyclopentyl or a cyclohexyl ring which may optionally be mono- or polysubstituted by CN, halogen, C₁-C₃-alkyl, C₁-C₃-alkoxy, C₁-C₃-haloalkyl, C₁-C₃-haloalkoxy or C₁-C₃-alkoxy-C₁-C₃-alkyl,
R¹² are identical or different and are hydrogen, C₁-C₆-alkyl, C₁-C₆-haloalkyl, unsubstituted or substituted C₃-C₆-cycloalkyl, C₁-C₄-trialkylsilyl, unsubstituted or substituted C₂-C₄-alkenyl, unsubstituted or substituted C₂-C₄-alkynyl, unsubstituted or substituted phenyl, C₁-C₄-alkoxy(C₁-C₄)alkyl, unsubstituted or substituted benzyl or a 3- to 7-membered unsubstituted or substituted, saturated or unsaturated cycle which may contain no or up to four heteroatoms selected from the group consisting of N, O and S, where two oxygen atoms are not adjacent,
or
if two radicals R¹² are attached to a nitrogen atom, two radicals R¹² may form a 3- to 7-membered unsubstituted or substituted saturated or unsaturated cycle which may contain up to four further heteroatoms selected from the group consisting of N, O and S, where two oxygen atoms are not adjacent,
or
if two radicals R¹² are adjacent in the grouping NR¹²COR¹², NR¹²SOR¹², NR¹²SO₂R¹², NR¹²SONR¹², NR¹²SO₂NR¹², two radicals R¹² may form a 3- to 7-membered unsubstituted or substituted saturated or unsaturated cycle which may contain up to four further heteroatoms selected from the group consisting of N, O and S, where two oxygen atoms are not adjacent,
R¹³ are identical or different and are C₁-C₈-alkyl, C₁-C₈-haloalkyl, C₁-C₄-trialkylsilyl, unsubstituted or substituted C₂-C₆-alkenyl, unsubstituted or substituted C₂-C₆-alkynyl, unsubstituted or substituted C₃-C₆-cycloalkyl, unsubstituted or substituted aryl, C₁-C₄-alkoxy-(C₁-C₄)alkyl, unsubstituted or substituted benzyl or a 3- to 7-membered unsubstituted or substituted saturated or unsaturated cycle which may contain no or up to four heteroatoms selected from the group consisting of N, O and S, where two oxygen atoms are not adjacent,
where two R¹³ may form a 3- to 7-membered unsubstituted or substituted saturated or unsaturated cycle which may contain up to four further heteroatoms selected from the group consisting of N, O and S, where two oxygen atoms are not adjacent,
and where possible substituents are selected from the list below:
fluorine, chlorine, bromine, iodine, cyano, nitro, CF₃, CFH₂, CF₂H, C₂F₅, CCl₃, hydroxyl, OMe, OEt, OPr, OisoPr, OBu, OsecBu, OisoBu, OtertBu, 0(CH₂)₂OCH₃, O(CH₂)₃OCH₃, O-cyclopentyl, O-phenyl, OCF₃, OCF₂H, OCF₂CF₃, OCF₂CF₂H, SH, SMe, SEt, SCF₃, SCF₂H, SPh, SCF₅, SO₂Me, SO₂CF₃, SOMe, SOEt, CO₂H, CO₂CH₃, CO₂Et, CO₂Pr, CO₂ isoPr, CO₂tertBu, COMe, COCF₃, NH₂, NHMe, NMe₂, NHEt, NEt₂, NHPr, NHisoPr, NHnBu, NHtertBu, NHisoBu, NHsecBu, cyclopropylamino, formyl, CH₂CN, CHMeCN, CH₂COCH₃, CH₂OMe, (CH₂)₂OMe, (CH₂)₃OMe, CH₂OH, CH₂SMe, (CH₂)₂SMe, methyl, ethyl, propyl, 1-methylethyl, butyl, 1-methylpropyl, 2-methylpropyl, 1,1-dimethylethyl, cyclopropyl, 1-methoxycyclopropyl, 1-chlorocyclopropyl, cyclobutyl, 3-dimethylbutyl, cyclopentyl, cyclohexyl, cyclohexylmethyl, neopentyl, prop-2-en-1-yl, 1-methylprop-2-en-1-yl, but-3-en-1-yl, (trimethylsilyl)methyl, phenyl, benzyl, —CH₂CH═CH, —CH(CH₃)CH═CH₂H or —CH₂C≡CH,
and agrochemically active salts thereof.

2. The method for controlling phytopathogenic harmful fungi comprising applying a compound of formula (I) according to claim 1
wherein:
X¹ represents nitrogen or CR³,
X² represents nitrogen or CR⁴,
where X¹ and X² do not both represent nitrogen,
R¹ and R⁵ independently of one another represent hydrogen, C₁-C₂-alkyl, C₁-C₂-alkoxy, F, Cl or Br,
R² represents hydrogen, halogen, nitro, cyano, C₁-C₄-alkyl, C₁-C₂-haloalkyl, hydroxyl, C₁-C₄-alkoxy, O—(C₂-C₄-alkenyl), O—(C₂-C₄-alkynyl), OCONH—(C₁-C₄-allyl), OCON—(C₁-C₄-alkyl)₂, C₁-C₃-haloalkoxy, COH, CO—(C₁-C₄-alkyl), C(═NOH)Me, C(═NO)—(C₁-C₄-alkyl)Me, CH(═NO)—(C₁-C₄-alkyl), SF₅, S—(C₁-C₃)-alkyl, S—(C₁-C₂)-haloalkyl, SO—(C₁-C₄-alkyl), SO₂—(C₁-C₄-alkyl), CO₂—(C₁-C₃-alkyl), NH₂, NH—(C₁-C₄-alkyl), N—(C₁-C₄-alkyl)₂, NHCO—(C₁-C₄-alkyl), NHCOH, NMeCO—(C₁-C₄-alkyl) or NHCO₂—(C₁-C₄-alkyl),
R³ and R⁴ independently of one another represent hydrogen, halogen, cyano, nitro, hydroxyl, O—(C₁-C₄)-alkyl, O—(C₃-C₆-cycloalkyl), O—(C₁-C₄-haloalkyl), OCONH—(C₁-C₄-alkyl), OCON—(C₁-C₄-alkyl)₂, OCO—(C₁-C₄-alkyl), SH, SF₅, S—(C₁-C₃-alkyl), S—(C₁-C₃-haloalkyl), SPh, SO—(C₁-C₄-alkyl), SO₂—(C₁-C₄-alkyl), SO₂—(C₂-C₄-alkenyl), SO₂—(C₂-C₄-alkynyl), SO₂—(C₁-C₂-haloalkyl), SO₂N—(C₁-C₄-alkyl)₂, formyl, CO—(C₁-C₄-alkyl), (C₁-C₃-haloalkyl)carbonyl, COCH₂CN, CONH—(C₁-C₄-alkyl), CON—(C₁-C₄-alkyl)₂, CONH—(C₁-C₃-haloalkyl), CONH—(C₂-C₄-alkenyl), CONH—(C₂-C₄-alkynyl), CONH—(C₃-C₆-cycloalkyl), CONHCH(CH₃)CH₂OCH₃, CONH(CH₂)ₘO—(C₁-C₄-alkyl), CONHPh, piperidin-1-ylcarbonyl, morpholin-4-ylcarbonyl, (4-methylpiperazin-1-yl)carbonyl, pyrrolidin-1-ylcarbonyl, azetidin-1-ylcarbonyl, aziridin-1-ylcarbonyl, hexamethylenimin-1-yl carbonyl, morpholin-1-ylcarbonyl, thiomorpholin-1-ylcarbonyl, COOH, (C₁-C₄-alkoxy)carbonyl, CO₂(CH₂)ₘO—(C₁-C₄-alkyl), NHCO—(C₁-C₄-alkyl), NHCO—(C₁-C₄-haloalkyl), N—(C₁-C₃-alkyl)-CO—(C₁-C₄-alkyl), N—(C₁-C₃-alkyl)-CS—(C₁-C₄-alkyl), NHCO—(C₂-C₄-alkenyl), NH(C═S)O—(C₁-C₄-alkyl), NHCOPh, NHCO(CH₂)ₘO—(C₁-C₄-alkyl), NHCHO, N—(C₁-C₄-alkyl)CHO, NHCO₂—(C₁-C₄-alkyl), NHCO₂Ph, NHCO₂—(C₁-C₂-haloalkyl), N—(C₁-C₄-alkyl)-CO₂—(C₁-C₄-alkyl), NH₂, NH—(C₁-C₄-alkyl), N—(C₁-C₂-alkyl)₂, cyclopropylamino, NHCH(CH₃)CH₂OCH₃, acetyl(cyclopropyl)amino, [(1-methylcyclopropyl)carbonyl]amino, morpholin-1-yl, morpholin-4-ylmethyl, NHSO₂—(C₁-C₄-alkyl), CH₂CN, CHMeCN, CH₂SO₂NH₄C₁-C₄-alkyl), CH₂SO₂N—(C₁-C₄-alkyl)₂, C(Me)HSO₂N—(C₁-C₄-alkyl)₂, CH₂CO—(C₁-C₄-alkyl), CH(CH₃)COCH₃, CH₂CO—(C₃-C₆-cycloalkyl), CH₂CONH—(C₁-C₄-alkyl), CH₂CO₂—(C₁-C₄-alkyl), CH₂NHCOO—(C₁-C₄-alkyl), CH═NO—(C₁-C₄-alkyl), C(CH₃)═NO—(C₁-C₄-alkyl), CH₂NHCO—(C₁-C₄-alkyl), CH₂NHCO—(C₁-C₄-haloalkyl), C₁-C₆-alkyl, C₃-C₆-cycloalkyl, 1-methoxycyclopropyl, 1-chlorocyclopropyl, C₂-C₆-alkenyl, C₁-C₃-haloalkyl, morpholin-4-ylsulphonyl, (3-methyl-2,5-dioxoimidozolidin-1-yl), 3-methyl-2-oxoimidazolidin-1-yl, (3-methyl-2-oxoimidazolidin-1-yl)methyl, piperidin-1-ylsulphonyl, 1,3-thiazol-2-yl, 1,3-thiazol-4-yl, 3,5-dimethylpiperidin-1-yl, 4-(tert-butoxycarbonyl)piperazin-1-yl, 5-ethoxy-3,4-dimethyl-1H-pyrazol-1-yl, acetyl(cyclohexyl)amino, 2-furoylamino, 5-ethoxy-3-(trifluoromethyl)-1H-pyrazol-1-yl, 3-(2-chloroethyl)-2-oxoimidazolidin-1-yl, 1,1-dioxidoisothiazolidin-2-yl, 1,1-dioxidotetrahydrothiophen-2-yl, 4-methyl-5-oxo-4,5-dihydro-1H-1,2,4-triazol-1-yl, 3-methyl-5-oxo-2,5-dihydro-1H-pyrazol-1-yl, 2-(methoxymethyl)pyrrolidin-1-yl, 2-oxocyclopentyl, 2-oxotetrahydrofuran-3-yl, 1-methyl-3-oxo-2,3-dihydro-1H-pyrazol-4-yl, 1-methyl-3-oxopyrazolidin-4-yl, tetrahydrofuran-2-yl, furan-2-yl, 5-methyl-1,1-dioxido-1,2,5-thiadiazolidin-2-yl, (benzylsulphamoyl)methyl, (furan-2-ylcarbonyl)amino, [(1-phenylethyl)sulphamoyl]methyl, 5-oxo-3-(propan-2-yl)-4,5-dihydro-1H-pyrazol-1-yl, 5-oxo-3-(trifluoromethyl)-4,5-dihydro-1H-pyrazol-1-yl, 1H-tetrazol-5-yl, (cyclopropylcarbonyl)amino, 4,4-dimethyl-2,5-dioxoimidazolidin-1-yl, 2,3-dimethyl-5-oxo-2,5-dihydro-1H-pyrazol-1-yl, 5-thioxo-4,5-dihydro-1H-tetrazol-1-yl, 3-(1-methylethyl)-2-oxoimidazolidin-1-yl, 3-(2-methylpropyl)-2-oxoimidazolidin-1-yl, 2-oxo-3-prop-2-en-1-ylimidazolidin-1-yl, 3-tert-butyl-2-oxoimidazolidin-1-yl, pyrrolidin-1-ylsulphonyl, 2,5-dioxoimidazolidin-4-yl, (morpholin-4-ylsulphonyl)methyl, (piperidin-1-ylsulphonyl)methyl, (pyrrolidin-1-ylsulphonyl)methyl, 2-oxoimidazolidin-1-yl, 3-methyl-5-oxo-4,5-dihydro-1H-pyrazol-1-yl, 3,4-dimethyl-5-oxo-4,5-dihydro-1H-pyrazol-1-yl, 1-methylcyclopentyl, pyrrolidin-1-yl, piperidin-1-yl, 2-oxo-2,5-dihydro-1H-pyrrol-1-yl, 3,3-dimethyl-2-oxocyclopentyl, 3-oxo-4,5-dimethyl-2,4-dihydro-pyrazol-2-yl, 3-oxo-4-ethyl-5-methyl-2,4-dihydropyrazol-2-yl, 3-oxo-5-trifluoromethyl-2,4-dihydropyrazol-2-yl, 3-oxo-5-isopropyl-2,4-dihydropyrazol-2-yl, 3,5-dioxo-4,4-dimethylpyrazolidin-1-yl, 3,5-dioxo-4-ethylpyrazolidin-1-yl, 3-oxo-4,4-dimethylpyrazolidin-1-yl, 2,5-dioxo-2,5-dihydro-1H-pyrrol-1-yl, 3-oxobutyl, 4-methoxy-2-oxo-2,5-dihydro-1H-pyrrol-1-yl, 2-oxo-2,5-dihydro-1H-pyrrol-1-yl, 5-oxo-4,5-dihydro-1H-imidazol-1-yl, 1,1-dioxido-1,2-thiazinan-2-yl, 6-methyl-1,1-dioxido-1,2,6-thiadiazinan-2-yl, cyclopropyl(trifluoroacetyl)amino, where m=1-3,
and, if R³ and R⁴, if appropriate via R¹² or R¹³, form a cycle, the following subunit from the general formula (I): 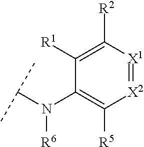
may be (2-oxo-2,3-dihydro-1H-indol-5-yl)amino, 1H-indol-6-ylamino, 1H-indol-5-ylamino, 2-(trifluoromethyl)-1H-benzimidazol-6-yl]amino, (3-methyl-1,1-dioxido-2H-1,2,4-benzothiadiazin-7-yl)amino, (1,1-dioxido-2H-1,2,4-benzothiadiazin-6-yl)amino, (4-methyl-3-oxo-3,4-dihydro-2H-1,4-benzoxazin-6-yl)amino, (4-methyl-3-oxo-3,4-dihydro-2H-1,4-benzoxazin-7-yl)amino, (1-acetyl-2,3-dihydro-1H-indol-6-yl)amino, (4H-1,3-benzodioxin-7-yl)amino, (2-oxo-2,3,4,5-tetrahydro-1H-1-benzazepin-8-yl)amino, (2,2-dioxido-1,3-dihydro-2-benzothien-5-yl)amino, (1-oxo-2,3-dihydro-1H-inden-5-yl)amino, [2-(ethylsulphonyl)-2,3-dihydro-1,3-benzothiazol-6-yl]amino, (2,2,3,3-tetrafluoro-2,3-dihydro-1,4-benzodioxin-6-yl)amino, 1,3-benzodioxol-5-ylamino, (1,3-dioxo-2,3-dihydro-1H-isoindol-5-yl)amino, (2-methyl-1,3-benzothiazol-6-yl)amino, (2-oxo-2,3-dihydro-1H-benzimidazol-5-yl)amino, (2-oxo-1,3-benzoxathiol-5-yl)amino, (2-oxo-2,3-dihydro-1,3-benzoxazol-5-yl)amino, (2-ethyl-1,3-benzoxazol-5-yl)amino, (2-oxo-1,2,3,4-tetrahydroquinolin-6-yl)amino, (3-oxo-3,4-dihydro-2H-1,4-benzoxazin-6-yl)amino, (2-oxo-2,3-dihydro-1,3-benzoxazol-6-yl)amino, 3-oxo-1,3-dihydro-2-benzofuran-5-yl)amino, [2-(ethylsulphonyl)-1,3-benzothiazol-6-yl]amino, (2-methyl-1,3-benzothiazol-5-yl)amino, (1-acetyl-2,3-dihydro-1H-indol-5-yl)amino, (2,2,3,3-tetrafluoro-2,3-dihydro-1,4-benzodioxin-6-yl)amino, (2,2-dioxido-1,3-dihydro-2-benzothiophen-5-yl)amino, (2-oxo-2,3-dihydro-1H-indol-6-yl)amino, (2-oxo-1,2,3,4-tetrahydroquinolin-7-yl)amino or 1H-indazol-6-ylamino,
R⁶ represents hydrogen, C₁-C₃-alkyl, CH₂OCH₃, CH₂CH₂OCH₃, formyl, (C₁-C₄-alkyl)carbonyl, (C₁-C₄-haloalkyl)carbonyl, (C₁-C₄-alkoxy)carbonyl, benzyloxycarbonyl, benzyl, 4-methoxybenzyl, 2,4-dimethoxybenzyl, 2-hydroxybenzyl, unsubstituted or substituted benzoyl, unsubstituted or substituted C₂-C₄-alkenyl, unsubstituted or substituted C₂-C₄-alkynyl, C₁-C₂-alkylsulphinyl or C₁-C₂-alkylsulphonyl,
where the substituents independently of one another are selected from the group consisting of hydrogen, fluorine, chlorine and bromine, C₁-C₂-alkyl, C₁-C₂-haloalkyl and cyano,
R⁷ represents hydrogen, methyl, cyano, CF₃, CFH₂ or CF₂H,
R⁸ represents chlorine, bromine, iodine, cyano, Me, SMe, SOMe, SO₂Me, CFH₂, CF₂H, or CF₃,
R⁹ represents hydrogen, methyl, ethyl, propan-1-yl, CH₂CH₂OMe or CH₂CH═CH₂,
R¹⁰ represents a group of the formula E1 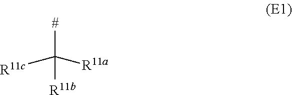
in which the symbols are as defined below and # represents the point of attachment to the nitrogen atom:
R^{11a} represents hydrogen or methyl,
R^{11b} represents hydrogen or C₁-C₃-alkyl,
R^{11c} represents hydrogen, C₁-C₄-alkyl, CN, CH₂CN, CH(Me)CN, C(Me)₂CN, CH₂OH, CH₂O—(C₁-C₃-alkyl), CH₂O—(C₁-C₃-haloalkyl), C(Me)HOH, C(Me)OH, C(Me)HO—(C₁-C₃-alkyl), C(Me)₂O—(C₁-C₃-alkyl), C(Me)HO—(C₁-C₂-haloalkyl), C(Me)₂O—(C₁-C₂-haloalkyl), CH₂S—(C₁-C₃-alkyl), C(Me)HS—(C₁-C₃-alkyl), C(Me)S—(C₁-C₃-alkyl), Si(C₁-C₂-alkyl)₃ or C₂-C₄-alkenyl which is optionally substituted by halogen or CF₃;
or
R¹⁰ represents a cyclopentyl or a cyclohexyl ring which may optionally be mono- or polysubstituted by CN, halogen, C₁-C₃-alkyl, C₁-C₃-alkoxy, C₁-C₃-haloalkyl, C₁-C₃-haloalkoxy or C₁-C₃-alkoxy-C₁-C₃-alkyl,
and also fungicidally active salts of these compounds.

3. The method for controlling phytopathogenic harmful fungi comprising applying a compound of formula (I) according to claim 1 wherein:
X¹ represents nitrogen or CR³,
X² represents nitrogen or CR⁴,
where X¹ and X² do not both represent nitrogen,
R¹ and R⁵ independently of one another represent hydrogen, C₁-C₂-alkyl, C₁-C₂-alkoxy, F or Cl,
R² represents hydrogen, fluorine, chlorine, bromine, iodine, nitro, cyano, C₁-C₃-alkyl, C₁-C₂-haloalkyl, hydroxyl, C₁-C₃-alkoxy, OCH₂CH═CH₂, OCH₂C≡CH, OCONHMe, C₁-C₂-haloalkoxy, COMe, COH, COEt, CMe(═NOH), CMe(═NOMe), SF₅, S—(C₁-C₃)-alkyl, SCF₃, SCF₂H, SOMe, SO₂Me, CO₂—(C₁-C₃-alkyl), NH₂, NH—(C₁-C₂-alkyl), N—(C₁-C₂-alkyl)₂, NHCO—(C₁-C₃-alkyl), NHCOH, NMeCO—(C₁-C₃-alkyl) or NHCO₂—(C₁-C₃-alkyl),
R³ and R⁴ independently of one another represent hydrogen, fluorine, chlorine, bromine, iodine, cyano, nitro, hydroxyl, O—(C₁-C₄-alkyl), O-cyclopentyl, OCF₃, OCF₂H, OCF₂CF₃, OCF₂CF₂H, OCONH—(C₁-C₃-alkyl), OCON—(C₁-C₃-alkyl)₂, OCO—(C₁-C₄-alkyl), SH, SF₅, S—C₁-C₃-alkyl, SCF₃, SCF₂H, SPh, SOMe, SO₂Me, SO₂CH₂CH═CH₂, SO₂CH₂CH═CH, SO₂CF₃, SO₂NMe₂, formyl, CO—(C₁-C₄-alkyl), COCF₃, COCH₂CN, CONH—(C₁-C₄-alkyl), CON—(C₁-C₄-alkyl)₂, CONHCH₂CF₃, CONHCH₂CH═CH₂, CONHCH₂C≡CH, CONHCH₂C(═CH₂)CH₃, CONHCH(CH₃)CH₂OCH₃, CONH(CH₂)₂OCH₃, CONHPh, CONH-cyclopropyl, piperidin-1-ylcarbonyl, morpholin-4-ylcarbonyl, (4-methylpiperazin-1-yl)carbonyl, COOH, (C₁-C₄-alkoxy)carbonyl, CO₂(CH₂)₂OCH₃, NHCO—(C₁-C₄-alkyl), NHCOCF₃, N(C₂H₅)COMe, NHCOCH═CH₂, NHCOPh, NHCOC(CH₃)₂CH₂F, NHCOC(CH₃)₂CH₂Cl, NHCO(C═CH₂)CH₃, NHCOCH₂OCH₃, NHCO(CH₂)₂OCH₃, N(CH₃)COCH₃, NHCHO, NMeCHO, NHCO₂—(C₁-C₄-alkyl), NHCO₂Ph, NHCO₂CH₂CH₂Cl, NMeCO₂Me, NH₂, NH—(C₁-C₄-alkyl), N—(C₁-C₂-alkyl)₂, cyclopropylamino, NHCH(CH₃)CH₂OCH₃, acetyl(cyclopropyl)amino, [(1-methylcyclopropyl)carbonyl]amino, morpholin-1-yl, morpholin-4-ylmethyl, NHSO₂Me, CH₂CN, CHMeCN, CH₂SO₂NH—(C₁-C₄-alkyl), CH₂SO₂N—(C₁-C₄-alkyl)₂, C(Me)HSO₂N—(C₁-C₄-alkyl)₂, CH₂COCH₃, CH₂COtertBu, CH(CH₃)COCH₃, CH₂COCH(CH₃)₂, CH₂CO-cyclopropyl, CH₂CONHtertBu, CH₂CO₂—(C₁-C₄-alkyl), CH₂NHCOOMe, CH═NOMe, C(CH₃)═NOMe, CH═NOEt, C(CH₃)═NOEt, CH₂NHAc, CH₂NHCOCF₃, C₁-C₄-alkyl, C₃-C₆-cycloalkyl, 1-methoxycyclopropyl, 1-chlorocyclopropyl, 3,3-dimethylbutyl, neopentyl, C₂-C₄-alkenyl, C₁-C₄-haloalkyl, morpholin-4-ylsulphonyl, 3-methyl-2,5-dioxoimidazolidin-1-yl, 3-methyl-2-oxoimidazolidin-1-yl, (3-methyl-2-oxoimidazolidin-1-yl)methyl, piperidin-1-ylsulphonyl, 1,3-thiazol-2-yl, 1,3-thiazol-4-yl, 3,5-dimethylpiperidin-1-yl, 4-(tert-butoxycarbonyl)piperazin-1-yl, 5-ethoxy-3,4-dimethyl-1H-pyrazol-1-yl, acetyl(cyclohexyl)amino, 2-furoylamino, (2,2,2-trifluoroethyl)carbamoyl, 5-ethoxy-3-(trifluoromethyl)-1H-pyrazol-1-yl, 3-(2-chloroethyl)-2-oxoimidazolidin-1-yl, 1,1-dioxidotetrahydrothiophen-2-yl, 4-methyl-5-oxo-4,5-dihydro-1H-1,2,4-triazol-1-yl, 3-methyl-5-oxo-2,5-dihydro-1H-pyrazol-1-yl, 2-(methoxymethyl)pyrrolidin-1-yl, 2-oxocyclopentyl, 2-oxotetrahydrofuran-3-yl, 1-methyl-3-oxo-2,3-dihydro-1H-pyrazol-4-yl, 1-methyl-3-oxopyrazolidin-4-yl, tetrahydrofuran-2-yl, furan-2-yl, 5-methyl-1,1-dioxido-1,2,5-thiadiazolidin-2-yl, (2,2-dimethylpropanoyl)(methyl)amino, (dimethylsulphamoyl)methyl, (benzylsulphamoyl)methyl, (furan-2-ylcarbonyl)amino, [(1-phenylethyl)sulphamoyl]methyl, 5-oxo-3-(propan-2-yl)-4,5-dihydro-1H-pyrazol-1-yl, 5-oxo-3-(trifluoromethyl)-4,5-dihydro-1H-pyrazol-1-yl, (cyclopropylcarbonyl)amino, 4,4-dimethyl-2,5-dioxoimidazolidin-1-yl, 3-(1-methylethyl)-2-oxoimidazolidin-1-yl, 3-(2-methylpropyl)-2-oxoimidazolidin-1-yl, 2-oxo-3-prop-2-en-1-ylimidazolidin-1-yl, 3-tert-butyl-2-oxoimidazolidin-1-yl, pyrrolidin-1-ylsulphonyl, 2,5-dioxoimidazolidin-4-yl, (morpholin-4-ylsulphonyl)-methyl, (piperidin-1-ylsulphonyl)methyl, (pyrrolidin-1-ylsulphonyl)methyl, 2-oxoimidazolidin-1-yl, 3-methyl-5-oxo-4,5-dihydro-1H-pyrazol-1-yl, 3,4-dimethyl-5-oxo-4,5-dihydro-1H-pyrazol-1-yl, 1-methylcyclopentyl, pyrrolidin-1-yl, piperidin-1-yl, 2-oxo-2,5-dihydro-1H-pyrrol-1-yl, 3,3-dimethyl-2-oxocyclopentyl, 3-oxo-4,5-dimethyl-2,4-dihydropyrazol-2-yl, 3-oxo-4-ethyl-5-methyl-2,4-dihydropyrazol-2-yl, 3,5-dioxo-4-ethylpyrazolidin-1-yl, 3-oxo-4,4-dimethylpyrazolidin-1-yl, 2,5-dioxo-2,5-dihydro-1H-pyrrol-1-yl, 4-methoxy-2-oxo-2,5-dihydro-1H-pyrrol-1-yl, 2-oxo-2,5-dihydro-1H-pyrrol-1-yl, 5-oxo-4,5-dihydro-1H-imidazol-1-yl, 1,1-dioxido-1,2-thiazinan-2-yl, 6-methyl-1,1-dioxido-1,2,6-thiadiazinan-2-yl or cyclopropyl(trifluoroacetyl)amino,
and, if R³ and R⁴, optionally via R¹² or R¹³, form a cycle, the following subunit from the general formula (I): 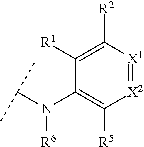
may be (2-oxo-2,3-dihydro-1H-indol-5-yl)amino, 1H-indol-6-ylamino, 1H-indol-5-ylamino, (4-methyl-3-oxo-3,4-dihydro-2H-1,4-benzoxazin-6-yl)amino, (4-methyl-3-oxo-3,4-dihydro-2H-1,4-benzoxazin-7-yl)amino, (1-acetyl-2,3-dihydro-1H-indol-6-yl)amino, (1-acetyl-2,3-dihydro-1H-indol-5-yl)amino, (2-oxo-2,3,4,5-tetrahydro-1H-1-benzazepin-8-yl)amino, 1,3-benzodioxol-5-ylamino, (1,3-dioxo-2,3-dihydro-1H-isoindol-5-yl)amino, (2-oxo-2,3-dihydro-1,3-benzoxazol-5-yl)amino, (2-ethyl-1,3-benzoxazol-5-yl)amino, (2-oxo-1,3-benzoxathiol-5-yl)amino, (2-oxo-1,2,3,4-tetrahydroquinolin-6-yl)amino, (3-oxo-3,4-dihydro-2H-1,4-benzoxazin-6-yl)amino, (2-oxo-2,3-dihydro-1,3-benzoxazol-6-yl)amino, [2-(ethylsulphonyl)-1,3-benzothiazol-6-yl]amino, (2-methy 1-1,3-benzothiazol-5-yl)amino, (2,2,3,3-tetrafluoro-2,3-dihydro-1,4-benzodioxin-6-yl)amino, (3-oxo-1,3-dihydro-2-benzofur-5-yl)amino, (2,2-dioxido-1,3-dihydro-2-benzothiophen-5-yl) or (1-oxo-2,3-dihydro-1H-inden-5-yl)amino,
R⁶ represents hydrogen, Me, ethyl, propanyl, formyl, CH₂OCH₃, COMe, COCF₃, COOMe, COOEt, COOtertBu, benzyloxycarbonyl, benzyl, benzoyl or 2-methylbenzoyl,
R⁷ represents hydrogen, methyl or CF₃,
R⁸ represents chlorine, bromine, CF₃, Me, cyano, iodine, CFH₂ CF₂H, SMe, SOMe or SO₂Me,
R⁹ represents hydrogen, methyl, ethyl, propan-1-yl, CH₂CH₂OMe or CH₂CH═CH₂,
R¹⁰ represents a group of the formula E1 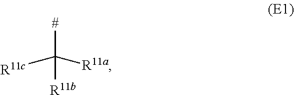
in which the symbols are as defined below and # represents the point of attachment to the nitrogen atom:
R^{11a} represents hydrogen or methyl,
R^{11b} represents hydrogen or C₁-C₃-alkyl,
R^{11c} represents hydrogen, C₁-C₄-alkyl, CN, CH₂CN, CH(Me)CN, CH₂OH, CH₂O—(C₁-C₂-alkyl), CH₂O—(C₁-C₂-haloalkyl), C(Me)HOH, C(Me)₂OH, C(Me)HO—(C₁-C₂-alkyl), C(Me)₂O—(C₁-C₂-alkyl), CH₂S—(C₁-C₂-alkyl), C(Me)HS—(C₁-C₂-alkyl), C(Me)₂S—(C₁-C₂-alkyl), SiMe₃ or C₂-C₄-alkenyl which is optionally substituted by halogen or CF₃;
Or
R¹⁰ represents a cyclopentyl or a cyclohexyl ring which may optionally be mono- or polysubstituted by CN, halogen, Me, Et, OMe, CF₃, OCF₃ or CH₂OCH₃,
and fungicidally active salts of these compounds.

4. The method for controlling phytopathogenic harmful fungi comprising applying a compound of formula (I) according to claim 1 wherein:
X¹ represents CR³,
X² represents nitrogen or CR⁴,
R¹ and R⁵ independently of one another represent hydrogen, methyl, methoxy or F,
R² represents hydrogen, fluorine, chlorine, bromine, cyano, methyl, ethyl, propanyl, propan-2-yl, CF₃, hydroxyl, OMe, OEt, OCF₃, COMe, COEt, SMe, SEt, CO₂CH₃, CO₂Et, NHMe, NMe₂, NHEt, NEt₂ or NHCOMe,
R³ and R⁴ independently of one another represent hydrogen, fluorine, chlorine, bromine, iodine, cyano, nitro, hydroxyl, OMe, OEt, OPr, OisoPr, OBu, OsecBu, OisoBu, OtertBu, O-cylopentyl, OCF₃, OCF₂CF₃, OCF₂CF₂H, OCONHMe, OCONHEt, OCONHPr, OCONHisoPr, OCONMe₂, OCONEt₂, OCONPr₂, OCONisoPr₂, OCOMe, OCOEt, OCOBu, SH, SF₅, SMe, SEt, SPr, SiPr, SCF₃, SCF₂H, SPh, SO₂Me, SO₂CF₃, SO₂NMe₂, formyl, COMe, COEt, COPr, COisoPr, COCF₃, COCH₂CN, CONHMe, CONMe₂, CONHEt, CONHCH₂CF₃, CONEt₂, CONHPr, CONHisoPr, CONHBu, CONHtertBu, CONHCH₂CH═CH₂, CONHCH₂C≡CH, CONHCH₂C(═CH₂)CH₃, CONHCH(CH₃)CH₂OCH₃, CONH(CH₂)₂OCH₃, CONHPh, CONH-cyclopropyl, piperidin-1-ylcarbonyl, morpholin-4-ylcarbonyl, COOH, CO₂CH₃, CO₂Et, CO₂Pr, CO₂ isoPr, CO₂(CH₂)₂OCH₃, NHCOMe, NHCOEt, NHCOPr, NHCOisoPr, NHCOBu, NHCOisoBu, NHCOCF₃, NHCOsecBu, NHCOtertBu, N(C₂H₅)COMe, NHCOCH═CH₂, NHCOPh, NHCOC(CH₃)₂CH₂F, NHCOC(CH₃)₂CH₂Cl, NHCO(C═CH₂)CH₃, NHCOCH₂OCH₃, NHCO(CH₂)₂OCH₃, N(CH₃)COCH₃, NHCHO, NMeCHO, NHCO₂Me, NHCO₂Et, NHCO₂ isoPr, NHCO₂tertBu, NHCO₂ isoBu, NHCO₂secBu, NHCO₂nBu, NHCO₂Ph, NHCO₂CH₂CH₂Cl, NMeCO₂Me, NH₂, NHMe, NMe₂, NHEt, NEt₂, NHPr, NHisoPr, NHnBu, cyclopropylamino, NHCH(CH₃)CH₂OCH₃, acetyl(cyclopropyl)amino, [(1-methylcyclopropyl)carbonyl]amino, morpholin-1-yl, morpholin-4-ylmethyl, NHSO₂Me, CH₂CN, CHMeCN, CH₂SO₂NHMe, CH₂SO₂NHPr, CH₂COCH₃, CH₂COtertBu, CH(CH₃)COCH₃, CH₂COCH(CH₃)₂, CH₂CO-cyclopropyl, CH₂CONHtertBu, CH₂CO₂Et, CH₂CO₂Me, CH₂NHCOOMe, CH₂NHAc, CH₂NHCOCF₃, methyl, ethyl, propyl, 1-methylethyl, butyl, 1-methylpropyl, 2-methylpropyl, 1,1-dimethylethyl, cyclopropyl, 1-methoxycyclopropyl, 1-chlorocyclopropyl, cyclobutyl, 3-dimethylbutyl, cyclopentyl, cyclohexyl, neopentyl, prop-2-en-1-yl, 1-methylprop-2-en-1-yl, but-3-en-1-yl, CF₃, CF₂H, morpholin-4-ylsulphonyl, 3-methyl-2,5-dioxoimidazolidin-1-yl, 3-methyl-2-oxoimidazolidin-1-yl, (3-methyl-2-oxoimidazo din-1-yl)methyl, piperidin-1-ylsulphonyl, 1,3-thiazol-2-yl, 1,3-thiazol-4-yl, 3,5-dimethylpiperidin-1-yl, 4-(tert-butoxycarbonyl)piperazin-1-yl, acetyl(cyclohexyl)amino, 2-furoylamino, 3-methyl-5-oxo-2,5-dihydro-1H-pyrazol-1-yl, tetrahydrofuran-2-yl, furan-2-yl, 5-methy 1-1,1-dioxido-1,2,5-thiadiazolidin-2-yl, (2,2-dimethyl-propanoyl)(methyl)amino, (benzylsulphamoyl)methyl, (dimethylsulphamoyl)methyl, (furan-2-ylcarbonyl)amino, [(1-phenylethyl)sulphamoyl]methyl, [ethyl(methyl)sulphamoyl]methyl, [methyl(propan-2-yl)sulphamoyl]methyl, 5-oxo-3-(propan-2-yl)-4,5-dihydro-1H-pyrazol-1-yl, 5-oxo-3-(trifluoromethyl)-4,5-dihydro-1H-pyrazol-1-yl, (cyclopropylcarbonyl)amino, 3-(1-methylethyl)-2-oxoimidazolidin-1-yl, 3-(2-methylpropyl)-2-oxoimidazolidin-1-yl, 2-oxo-3-prop-2-en-1-ylimidazolidin-1-yl, 3-tert-butyl-2-oxoimidazolidin-1-yl, pyrrolidin-1-ylsulphonyl, 2,5-dioxoimidazolidin-4-yl, (morpholin-4-ylsulphonyl)methyl, (piperidin-1-ylsulphonyl)methyl, (pyrrolidin-1-ylsulphonyl)methyl, 2-oxoimidazolidin-1-yl, 3,3-dimethyl-2-oxocyclopentyl, 3-oxo-4,5-dimethyl-2,4-dihydropyrazol-2-yl, 3,5-dioxo-4-ethylpyrazolidin-1-yl, 5-oxo-4,5-dihydro-1H-imidazol-1-yl, 1,1-dioxido-1,2-thiazinan-2-yl, 6-methyl-1,1-dioxido-1,2,6-thiadiazinan-2-yl or cyclopropyl(trifluoroacetyl)amino,
and, if R³ and R⁴, if appropriate via R¹² or R¹³, form a cycle, the following subunit from the general formula (I): 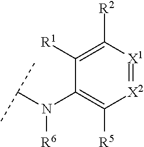
may represent (2-oxo-2,3-dihydro-1H-indol-5-yl)amino, 1H-indol-6-ylamino, 1H-indol-5-ylamino, (4-methyl-3-oxo-3,4-dihydro-2H-1,4-benzoxazin-6-yl)amino, (4-methyl-3-oxo-3,4-dihydro-2H-1,4-benzoxazin-7-yl)amino, (1-acetyl-2,3-dihydro-1H-indol-6-yl)amino, (1-acetyl-2,3-dihydro-1H-indol-5-yl)amino, (2-oxo-2,3,4,5-tetrahydro-1H-1-benzazepin-8-yl)amino, 1,3-benzodioxol-5-ylamino, (2-oxo-1,3-benzoxathiol-5-yl)amino, (2-oxo-1,2,3,4-tetrahydroquinolin-6-yl)amino, (3-oxo-3,4-dihydro-2H-1,4-benzoxazin-6-yl)amino, (2-oxo-2,3-dihydro-1,3-benzoxazol-6-yl)amino, (2-methyl-1,3-benzothiazol-5-yl)amino, (2,2,3,3-tetrafluoro-2,3-dihydro-1,4-benzodioxin-6-yl)amino, (3-oxo-1,3-dihydro-2-benzofur-5-yl)amino or 1-oxo-2,3-dihydro-1H-inden-5-yl)amino,
R⁶ represents hydrogen, Me, formyl or COMe,
R⁷ represents hydrogen,
R⁸ represents chlorine, bromine, CF₃, Me, cyano, iodine or CFH₂,
R⁹ represents hydrogen, methyl, ethyl, propan-1-yl, CH₂CH₂OMe or CH₂CH═CH₂,
R¹⁰ represents methyl, ethyl, propanyl, propan-2-yl, butanyl, butan-2-yl, 2-methylpropanyl, tert-butyl, pentanyl, 3-methylbutanyl, 2-methylbutanyl, 2,2-dimethylpropanyl, pentan-2-yl, pentan-3-yl, 4-methylpentan-2-yl, hexan-3-yl, 3,3-dimethylbutan-2-yl, 2,4-dimethylpentan-3-yl, cyclopentyl, 2-methyl cyclopentyl, 2-ethylcyclopentyl, 2-chlorocyclopentyl, 2-fluorocyclopentyl, 2-cyanocyclopentyl, 2-methoxycyclopentyl, 2-(methoxymethyl)cyclopentyl, cyclohexyl, 2-methoxycyclohexyl, 2-methylcyclohexyl, 4,4-difluorocyclohexyl, 4-(trifluoromethyl)cyclohexyl, prop-2-en-1-yl, 2-methylprop-2-en-1-yl, 1-methylprop-2-en-1-yl, 3-methylbut-2-en-1-yl, but-2-en-1-yl, 4,4,4-trifluorobut-2-en-1-yl, 3-chlorobut-2-en-1-yl, 2-chloroprop-2-en-1-yl, cyanomethyl, 2-cyano ethyl, 1-cyanopropan-2-yl, 3-cyanobutan-2-yl, (trimethylsilyl)methyl, 1-(trimethylsilyl)ethyl, 2-hydroxyethyl, 2-methoxyethyl, 2-ethoxyethyl, 2-(trifluoromethoxy)ethyl, 1-hydroxypropan-2-yl, 1-hydroxybutan-2-yl, 3-hydroxy-3-methylbutan-2-yl, 2-hydroxy-2-methylpropanyl, 3-methoxy-3-methylbutan-2-yl, 2-methoxy-2-methylpropanyl, 1-methoxypropan-2-yl, 1-ethoxypropan-2-yl, 1-trifluoromethoxypropan-2-yl, 1-methoxybutan-2-yl, 3-methoxybutan-2-yl, 1-methoxy-3-methylbutan-2-yl, 1-methoxypentan-2-yl, 2-(methylsulphanyl)ethyl, (2R)-1-(methylsulphanyl)propan-2-yl, (2S)-1-(methylsulphanyl)propan-2-yl or 2-methyl-1-(methylsulphanyl)propan-2-yl,
and agrochemically active salts thereof.

5. A compound of formula (Ia), 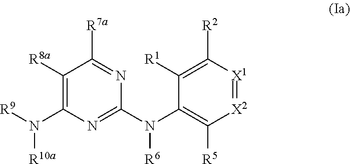
in which
X¹, X², R¹ to R⁶, R⁹, R¹² and R¹³ are as defined in claim 1,
R^{7a} represents hydrogen, C₂-C₃-alkyl, cyano or C₁-C₃-haloalkyl,
R^{8a} represents chlorine, iodine, SMe, SOMe, SO₂Me, CCl₃, CFH₂ or CF₂H,
R^{10a} represents a group E1 of the formula 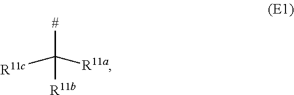
in which the symbols are as defined below and # represents the point of attachment to the nitrogen atom:
R^{11a} represents hydrogen or methyl,
R^{11b} represents hydrogen or C₁-C₃-alkyl,
R^{11c} represents hydrogen, C₁-C₄-alkyl, CN, CH₂CN, CH(Me)CN, C(Me)₂CN, CH₂O—(C₁-C₃-alkyl), CH₂O—(C₁-C₃-haloalkyl), C(Me)₂OH, C(Me)HO—(C₁-C₃-alkyl), C(Me)₂O—(C₁-C₃-alkyl), C(Me)HO—(C₁-C₃-haloalkyl), C(Me)₂O—(C₁-C₃-haloalkyl), CH₂S—(C₁-C₃-alkyl), C(Me)HS—(C₁-C₃-alkyl), C(Me)₂S—(C₁-C₃-alkyl), Si—(C₁-C₂-alkyl)₃ or C₂-C₄-alkenyl which is optionally substituted by halogen or CF₃,
or
R^{11c} represents a cyclopentyl or a cyclohexyl ring which is optionally substituted by CN, halogen, C₁-C₃-alkyl, C₁-C₃-alkoxy, C₁-C₃-haloalkyl, C₁-C₃-haloalkoxy or C₁-C₃-alkoxy-C₁-C₃-alkyl,
and agrochemically active salts thereof.

6. A compound of formula (Ib) 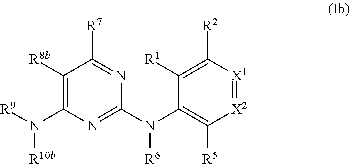
in which
X¹, X², R¹ to R⁷, R⁹, R¹² and R¹³ are as defined in claim 1,
R^{8b} represents CF₃,
R^{10b} represents a group E1 of the formula below 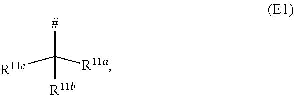
in which the symbols are as defined below and # represents the point of attachment to the nitrogen atom:
R^{11a} represents hydrogen or methyl,
R^{11b} represents hydrogen or C₁-C₃-alkyl,
R^{11c} represents hydrogen, C₁-C₄-alkyl, CN, CH₂CN, CH(Me)CN, C(Me)₂CN, CH₂O—(C₁-C₃-alkyl), CH₂O—(C₁-C₃-haloalkyl), C(Me)₂OH, C(Me)HO—(C₁-C₃-alkyl), C(Me)₂O—(C₁-C₃-alkyl), C(Me)HO—(C₁-C₃-haloalkyl), C(Me)₂O—(C₁-C₃-haloalkyl), CH₂S—(C₁-C₃-alkyl), C(Me)HS—(C₁-C₃-alkyl), C(Me)₂S—(C₁-C₃-alkyl), Si—(C₁-C₂-alkyl)₃ or C₂-C₄-alkenyl which is optionally substituted by halogen or CF₃,
or
R^{11c} represents a cyclopentyl or a cyclohexyl ring which is optionally substituted by CN, halogen, C₁-C₃-alkyl, C₁-C₃-alkoxy, C₁-C₃-haloalkyl, C₁-C₃-haloalkoxy or C₁-C₃-alkoxy-C₁-C₃-alkyl;
provided that R¹, R², R⁵, X¹, and X² do not have the meaning below 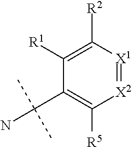
X¹ represents CR³ and X² represents CR⁴ and
R³ and R⁴ together with the phenyl ring form a (1H-2-hydroxyindol-5-yl) or a (1H-2-hydroxyindol-6-yl) radical;
or R³ represents CON(R¹²)₂ and the two radicals R¹² together with the nitrogen atom to which they are attached form a 4-methyl-1,4-piperazinyl radical;
or R² and R³ or R³ and R⁴ represent chlorine;
and agrochemically active salts thereof.

7. A compound of formula (Ic), 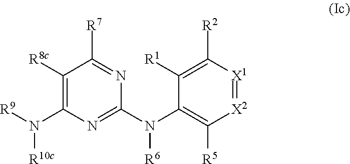
in which
X¹, X², R¹ to R⁷, R⁹, R¹² and R¹³ are as defined in claim 1,
R^{8c} represents bromine and
R^{10c} represents a group E1 of the formula 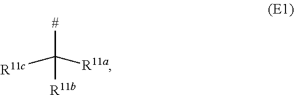
in which the symbols are as defined below and # represents the point of attachment to the nitrogen atom:
R^{11a} represents hydrogen or methyl,
R^{11b} represents hydrogen or C₁-C₃-alkyl,
R^{11c} represents hydrogen, C₁-C₄-alkyl, CN, CH₂CN, CH(Me)CN, C(Me)₂CN, CH₂O—(C₁-C₃-alkyl), CH₂O—(C₁-C₃-haloalkyl), C(Me)₂OH, C(Me)HO—(C₁-C₃-alkyl), C(Me)₂O—(C₁-C₃-alkyl), C(Me)HO—(C₁-C₃-haloalkyl), C(Me)₂O—(C₁-C₃-haloalkyl), CH₂S—(C₁-C₃-alkyl), C(Me)HS—(C₁-C₃-alkyl), C(Me)₂S—(C₁-C₃-alkyl), Si—(C₁-C₂-alkyl)₃ or C₂-C₄-alkenyl which is optionally substituted by halogen or CF₃,
or
R^{11c} represents a cyclopentyl or a cyclohexyl ring which is optionally substituted by CN, halogen, C₁-C₃-alkyl, C₁-C₃-alkoxy, C₁-C₃-haloalkyl, C₁-C₃-haloalkoxy or C₁-C₃-alkoxy-C₁-C₃-alkyl;
provided that R1, R2, R5, X1, and X2 do not have the meaning below 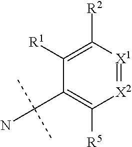
X¹ represents CR³ and X² represents CR⁴ and
R³ and R⁴ together with the phenyl ring form a 1H-2-hydroxyindol-5-yl or a 1H-indazol-5-ylamino radical;
or R³ represents OH, SMe, SOMe, SO₂NMe₂, piperazin-1-yl, 4-tbutyloxycarbonylpiperazin-1-yl or (pyrrolidin-1-yl)methyl;
or R² or R⁴ represents SMe;
and the agrochemically active salts thereof.

8. A composition for controlling phytopathogenic harmful fungi, comprising at least one compound of formula (Ia) according to claim 5, in addition to one or more extenders, surfactants or a combination thereof.

9. (canceled)

10. A method for controlling phytopathogenic harmful fungi comprising applying the compound of formula (Ia) according to claim 5 to one or more phytopathogenic harmful fungi, their habitat or a combination thereof.

11. A process for preparing a composition for controlling phytopathogenic harmful fungi comprising mixing the compound of formula (Ia) according to claim 5 mixed with one or more extenders, surfactants or a combination thereof.

12. A process for preparing the compound of formula (Ia), (Ib) and (Ic) according to the invention, comprising at least one of steps (a) to (f) below:
(a) reacting a compound of formula (III) with a compound of formula (II) in the presence of a base, if appropriate in the presence of a solvent, if appropriate in the presence of a catalyst, to give a compound of formula (V), according to the reaction scheme below: 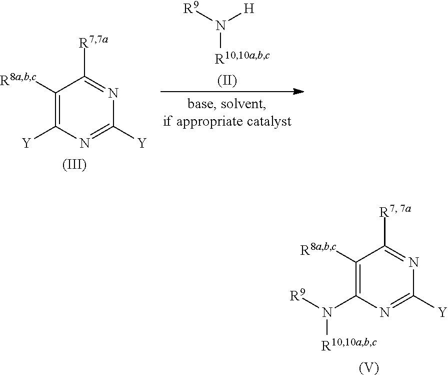
where Y═F, Cl, Br or I, (b) reacting the compound of formula (V) with a compound of formula (IV), if appropriate in the presence of an acid, if appropriate in the presence of a solvent, according to the reaction scheme below: 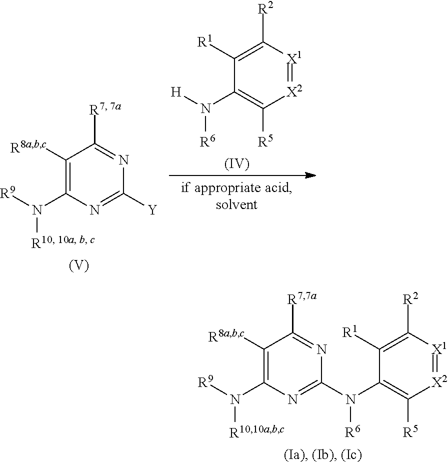
where Y═F, Cl, Br or I,
(c) reacting a compound of formula (VIb) with a the compound of the formula (IV), if appropriate in the presence of an acid and if appropriate in the presence of a solvent, according to the reaction scheme below: 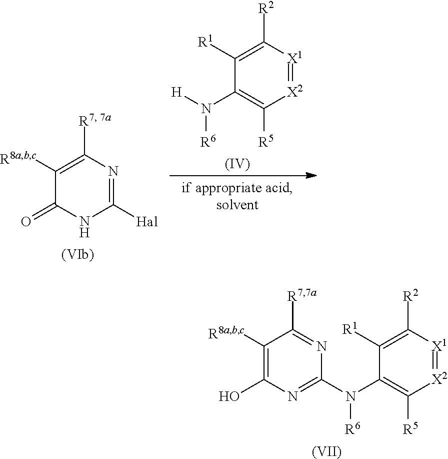
where Hal═F, Cl, Br or I,
(d) reacting a compound of formula (VII) with a halogenating agent, if appropriate in the presence of a solvent, to give a compound of the formula (VIII), according to the reaction scheme below: 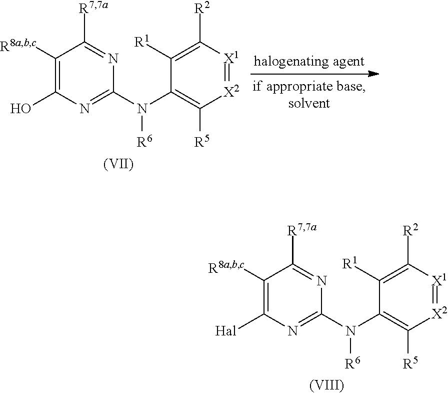
(e) reacting a compound of formula (IIIb) with the compound of the formula (IV), if appropriate in the presence of a catalyst and if appropriate in the presence of a solvent, according to the reaction scheme below: 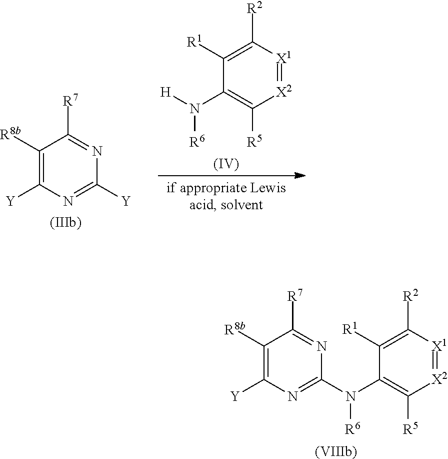
where Y═F, Cl, Br or I and R^{8b}═CF₃,
(f) reacting the compound of formula (VIII) with the compound of formula (II) in the presence of a base, if appropriate in the presence of a solvent, if appropriate in the presence of a catalyst, to give a compound of formula (Ia, Ib and Ic), according to the reaction scheme below: 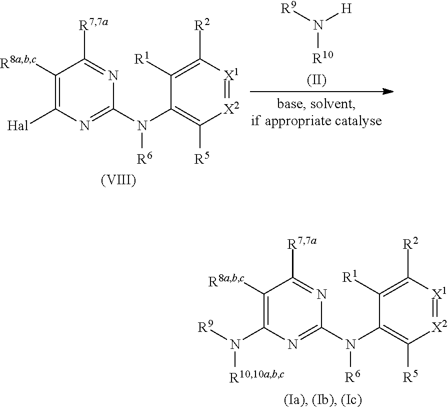
where the definitions of the radicals R¹ to R¹⁰ and X¹ and X² in the above schemes correspond to the definitions according to claim 1 and Y and Hal represents F, Cl, Br or I.

13. A compound of formula (Va) 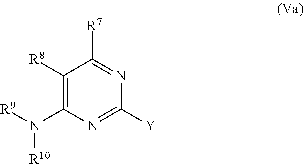
in which
R⁷ represents hydrogen,
R⁸ represents chlorine, iodine, SMe, SOMe, SO₂Me, CF₃, CCl₃, CFH₂ or CF₂H,
R⁹represents hydrogen, C₁-C₃-alkyl, C₁-C₄-alkoxy-C₁-C₄)alkyl, (C₁C₄alkyl)carbonyl, (C₁-C₄-haloalkyl)carbonyl, (C₁-C₄-alkoxy)carbonyl or optionally fluorine-, chlorine-, bromine-, cyano- or CF₃-substituted C₂-C₄-alkenyl,
Y═F, Cl, Br or I
and
R¹⁰ represents a group E1 of the formula 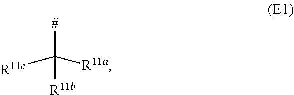
in which the symbols are as defined below and # represents the point of attachment to the nitrogen atom:
R^{11a} represents hydrogen or methyl,
R^{11b} represents hydrogen or C₁-C₃-alkyl,
R^{11c} represents C₂-C₄-alkyl, CN, CH₂CN, CH(Me)CN, C(Me)₂CN, CH₂O—(C₁-C₃-alkyl), CH₂O—(C₁-C₃-haloalkyl), C(Me)₂OH, C(Me)HO—(C₁-C₃-alkyl), C(Me)₂O—(C₁-C₃-alkyl), C(Me)HO—(C₁-C₃-haloalkyl), C(Me)₂O—(C₁-C₃-haloalkyl), CH₂S—(C₁-C₃-alkyl), C(Me)HS—(C₁-C₃-alkyl), C(Me)S—(C₁-C₃-alkyl), Si—(C₁-C₂-alkyl)₃ or C₂-C₄-alkenyl which is optionally substituted by halogen or CF₃,
or
R^{11c} represents a cyclopentyl ring which may optionally be mono- or polysubstituted by CN, halogen, C₁-C₃-alkyl, C₁-C₃-alkoxy, C₁-C₃-haloalkyl, C₁-C₃-haloalkoxy or C₁-C₃-alkoxy-C₁-C₃-alkyl,
Or
R^{11c} represents a cyclohexyl ring which is substituted by at least one of the radicals from the group consisting of CN, halogen, C₁-C₃-alkyl, C₁-C₃-alkoxy, C₁-C₃-haloalkyl, C₁-C₃-haloalkoxy or C₁-C₃-alkoxy-C₁-C₃-alkyl.

14. A composition for controlling phytopathogenic harmful fungi, comprising at least one compound of formula (Ib) according to claim 6, in addition to one or more extenders, surfactants or a combination thereof.

15. A composition for controlling phytopathogenic harmful fungi, comprising at least one compound of formula (Ic) according to claim 7, in addition to one or more extenders, surfactants or a combination thereof.

16. A method for controlling phytopathogenic harmful fungi comprising applying the compound of formula (Ib) according to claim 6 to one or more phytopathogenic harmful fungi, their habitat or a combination thereof.

17. A method for controlling phytopathogenic harmful fungi comprising applying the compound of formula (Ic) according to claim 7 to one or more phytopathogenic harmful fungi, their habitat or a combination thereof.

18. A process for preparing a composition for controlling phytopathogenic harmful fungi comprising mixing the compound of formula (Ib) according to claim 6 with one or more extenders, surfactants or a combination thereof.

19. A process for preparing a composition for controlling phytopathogenic harmful fungi comprising mixing the compound of formula (Ic) according to claim 7 with one or more extenders, surfactants or a combination thereof.
